SEQUENCE LISTING (14 sequences)

SEQ ID NO: 1 - DNA, 12513 bp, synthetic construct
  misc_feature 1..12513: note = pAAV ABCA4 3'RTM CMV synthetic construct
  source 1..12513: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtaagtatcaaggttacaagacaggtttaaggagaccaatagaaactgggcttgtcgagacagagaagactcttgcgtttctgataggcacctattggtcttactgacatccactttgcctttctctccacaggttggtgtacactagcggccgcaaactctgctacactcacacatgctttgtgtggctgtgggtttgataaaagttcatggaaggagctagttggtgcccaggctgacacatgtagaagagagacttctagaatccacaggaattttggtccccatgttttcaaagcccatacaagcttcgaattcgatatcgagaacattattatagcgttgctcgagtactaactggtacctcttcttttttttcgtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgactgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaagg

SEQ ID NO: 2 - DNA, 13227 bp, synthetic construct
  misc_feature 1..13227: note = pAAV CEP290 5'RTM CMV CBA Ex1_26 5'SS BD_5.1 synthetic DNA construct recombinant plasmid
  source 1..13227: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgcatgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatcttctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagaattagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggaggtaagagagctcgttgcgatattattacagatatccagcacagtggcggccgctgtaatcccagcactttaggaggccgaggcgggtggatcacgagttcaggagatcgacaccgcggttcgaaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaagg

SEQ ID NO: 3 - protein, 269 aa, Saccharomyces cerevisiae
MSHIQRETSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 4 - protein, 269 aa, Saccharomyces cerevisiae; identical to SEQ ID NO: 3

SEQ ID NO: 5 - DNA, 25 bp, synthetic construct
  misc_feature 1..25: note = 5' splice site with spacer
  source 1..25: mol_type = other DNA
gtaagagagctcgttgcgatattat

SEQ ID NO: 6 - DNA, 33 bp, synthetic construct
  misc_feature 1..33: note = splice site for a 3'RTM
  source 1..33: mol_type = other DNA
tactaactggtacctcttcttttttttctgcag

SEQ ID NO: 7 - DNA, 23 bp, synthetic construct
  misc_feature 1..23: note = polypyrrimidine tract for the 3' RTM
  source 1..23: mol_type = other DNA
tggtacctcttcttttttttctg

SEQ ID NO: 8 - DNA, 20 bp, synthetic construct
  misc_feature 1..20: note = Spacer for a 5' RTM
  source 1..20: mol_type = other DNA
agatctcgttgcgatattat

SEQ ID NO: 9 - DNA, 27 bp, synthetic construct
  misc_feature 1..27: note = Spacer for a 3' RTM
  source 1..27: mol_type = other DNA
gagaacattattatagcgttgctcgag

SEQ ID NO: 10 - DNA, 27 bp, synthetic construct
  misc_feature 1..27: note = 3' RTM Spacer sequence
  source 1..27: mol_type = other DNA
gagaacattattatagcgttgctcgag

SEQ ID NO: 11 - DNA, 23 bp, synthetic construct
  misc_feature 1..23: note = 3' RTM Polypyrimidine tract
  source 1..23: mol_type = other DNA
tggtacctcttcttttttttctg

SEQ ID NO: 12 - DNA, 20 bp, synthetic construct
  misc_feature 1..20: note = 5'RTM spacer sequence
  source 1..20: mol_type = other DNA
agagctcgttgcgatattat

SEQ ID NO: 13 - DNA, 20 bp, synthetic construct
  misc_feature 1..20: note = 3'RTM spacer sequence
  source 1..20: mol_type = other DNA
agagctcgttgcgatattat

SEQ ID NO: 14 - DNA, 23 bp, synthetic construct
  misc_feature 1..23: note = 3 'RTM Polypyrrimidine tract
  source 1..23: mol_type = other DNA
tggtacctcttcttttttttctg

(19) United States
(12) Patent Application Publication
(10) Pub. No.: US 2026/0191995 A1
(43) Pub. Date: Jul. 9, 2026

(54) Compositions and Methods for Correction of Heritable Ocular Disease

(71) Applicants: Lloyd G. Mitchell, Bethesda, MD (US); The Trustees of the University of Pennsylvania, Philadelphia, PA (US)

(72) Inventors: Jean Bennett, Bryn Mawr, PA (US); Jeannette Bennicelli, Philadelphia, PA (US); Scott Dooley, Clementon, PA (US); Lloyd G. Mitchell, Bethesda, MD (US)

(21) Appl. No.: 19/568,148

(22) Filed: Mar. 16, 2026

Related U.S. Application Data
(62) Division of application No. 17/230,354, filed on Apr. 14, 2021, now Pat. No. 12,616,757.
Application No. 17/230,354 is a continuation of application No. 15/776,663, filed on Nov. 18, 2016, now Pat. No. 10,987,433.
(60) Provisional application No. 62/257,500, filed on Nov. 19, 2015.

(51) Int. Cl. A61K 48/00; C12N 15/00; C12N 15/11; C12N 15/86

(52) U.S. Cl. CPC A61K 48/005; A61K 48/0075; C12N 15/00; C12N 15/11; C12N 15/86; C12N 2740/10043; C12N 2750/14143

(57) ABSTRACT

A nucleic acid trans-splicing molecule is provided that can replace an exon in a targeted mammalian ocular gene carrying a defect or mutation causing an ocular disease with an exon having the naturally-occurring sequence without the defect or mutation. A method of treating an ocular disease, e.g., Stargardt's Disease, caused by a defect or mutation in a target gene, e.g., ABCA4 comprising: administering to the ocular cells of a subject having an ocular disease a composition comprising a recombinant AAV comprising a nucleic acid trans-splicing molecule as described above.

### CROSS-REFERENCE TO RELATED APPLICATIONS

This application is a divisional of a U.S. patent application Ser. No. 17/230,354, filed Apr. 14, 2021, which claims benefit of U.S. Nonprovisional patent application Ser. No. 15/776,663, filed May 16, 2018, which is a national stage entry of International Patent Application No. PCT/US2016/062941, filed Nov. 18, 2016, which claims the benefit of the priority of U.S. Provisional Patent Application No. 62/257,500, filed Nov. 19, 2015, which applications are incorporated herein by reference.

### STATEMENT REGARDING FEDERALLY SPONSORED RESEARCH OR DEVELOPMENT

This invention was made with government support under Grant No. 1R24 EY019861-01A1 awarded by the National Institutes of Health. The government has certain rights in this invention.

### REFERENCE TO AN ELECTRONIC SEQUENCE LISTING

The electronic sequence listing filed herewith named “UPN-15-7313USD2.xml” with size of 44,443 bytes, created on date of Feb. 26, 2026, and the contents of the electronic sequence listing (e.g., the sequences and text therein) are incorporated herein by reference in entirety.

### BACKGROUND

A number of inherited retinal diseases are caused by mutations, generally multiple mutations, located throughout portions of large ocular genes. As one example, Stargardt disease, also known as Stargardt 1 (STGD1), is an autosomal recessive form of retinal dystrophy that is usually characterized by a progressive loss of central vision. Worldwide prevalence of STGD1 is estimated at 1/8,000-1/10,000. The disease typically presents within the first two decades of life. Although disease progression and severity varies widely, STGD1 is usually characterized by a progressive loss of central vision causing blurry vision and, occasionally, an increasing difficulty to adapt in the dark. STGD1 may progress rapidly over a few months or gradually over several years leading to a severe decrease in visual acuity. Most affected individuals also have impaired color vision or photophobia. There is no treatment currently available for STGD1.

STGD1 has been linked to mutations in the ABCA4 gene, which has a sequence of 6822 nucleotides that encodes an adenosine triphosphate (ATP)-binding cassette transporter (ABCR) of sub-family A number 4, which is expressed specifically in the cones and rods of the retina. Defects in ABCR function cause the accumulation of all-trans-retinal and its cytotoxic derivatives (e.g., diretinoid-pyridinium-ethanolamine) (lipofuscin pigments) in photoreceptors and retinal pigment epithelial (RPE) cells, ultimately causing RPE cell death and the subsequent loss of photoreceptors. Mutations in ABCA4 have been linked to a spectrum of phenotypes ranging from STGD1, to a juvenile onset macular degeneration, fundus flavimaculatus, to cone-rod dystrophy, and a form of retinitis pigmentosa. ABCA4 mutations also contribute to age-related macular degeneration (AMD) and severe early-onset retinal dystrophy.

Similar retinal diseases are caused by defects in other large ocular genes, including CEP290 (7440 nucleotides) which defects or mutations cause Leber's congenital amaurosis, among other ocular disorders, and MYO7A (7465 nucleotides), which defects or mutations cause Usher's disease.

The occurrences and locations of multiple mutations in such large ocular genes has made strategies for repairing the mutations very challenging. There remains a need for effective compositions and therapeutic methods for treating such ocular disorders.

### SUMMARY

In one aspect, a composition comprises a pre-RNA trans-splicing molecule (RTM) that can replace an exon or multiple exons in a targeted mammalian ocular gene carrying a defect or mutation causing an ocular disease with an exon(s) having the naturally-occurring sequence without the defect or mutation.

In another aspect, a recombinant nucleic acid molecule and vectors capable of expressing the RTMs described herein are provided.

In still another aspect, ocular cells expressing the RTM are provided for use in ex vivo repair and reimplantation to the subject from which the ocular cells were extracted.

In another aspect, a proviral plasmid comprises a modular recombinant AAV genome comprising in operative association comprising a 5′ AAV2 ITR sequence, a suitable promoter operative in a mammalian ocular cell, an RNA trans-splicing molecule that can replace an exon in a targeted mammalian ocular gene carrying a defect or mutation causing an ocular disease with an exon having the naturally-occurring sequence without the defect or mutation, wherein the RTM is operatively linked to, and under the regulatory control of, the promoter; and a 3′ AAV2 ITR sequence. The modular AAV genome is present in a plasmid backbone comprising the elements necessary for replication in a host cell.

In yet another aspect, a cell culture comprises bacterial or mammalian host cells transfected with the plasmids or nucleic acid constructs described herein.

In another aspect, a recombinant AAV infectious particle comprises an RTM or nucleic acid construct described herein.

In another embodiment, a recombinant AAV infectious particle is produced by culturing a packaging cell carrying a proviral plasmid as described herein and carrying an RTM in the presence of sufficient viral sequences to permit packaging of the ocular gene nucleic acid sequence expression cassette viral genome into an infectious AAV envelope or capsid.

In one aspect, a kit is provided that comprises an RTM as described herein, a recombinant nucleic acid construct as described herein, or a proviral plasmid as described herein.

In another aspect, a method of treating an ocular disease caused by a defect or mutation in a target gene comprising administering to the ocular cells of a mammalian subject having the ocular disease a composition comprising an rAAV particle carrying an RNA trans-splicing molecule (RTM) that can replace an exon in a targeted mammalian ocular gene carrying a defect or mutation causing an ocular disease with an exon having the naturally-occurring sequence without the defect or mutation. These methods include ex vivo methods including contacting the RTMs with specific target pre-mRNA expressed within ocular cells under conditions in which a portion of the RTM is trans-spliced to a portion of the target pre-mRNA to form a chimeric RNA molecule which contains sequence in which the genetic defect in the specific target ocular gene is corrected for return to the subject's eye.

In another aspect, the method of treatment involves administering via sub-retinal injection to the ocular cells an rAAV particle comprising the RTM, wherein the ocular cell infected with the rAAV employs the RTM to replace the defective gene in vivo by trans-splicing.

Other aspects and embodiments are described in the following detailed description.

### BRIEF DESCRIPTION OF THE FIGURES

FIG. 1 shows a diagram of an AAV genome encoding RNA trans-splicing molecules targeting mutations in ABCA-4.

FIG. 2A is a 5′RTM model for CEP290 (Exons 1 to 26) inserted into the p618 plasmid.

FIG. 2B is a linearized map focusing on the provirus containing the RTM of FIG. 2A, i.e., the plasmid bases only between the 5′ and 3′ AAV ITRs.

FIG. 3A is a 3′RTM prophetic model for ABCA4 (Exons 27-50) inserted into a modified shorter-intron p618 plasmid.

FIG. 3B is a linearized map of the focusing on the provirus containing the RTM of FIG. 3A, i.e., the plasmid bases only between the 5′ and 3′ AAV ITRs.

### DETAILED DESCRIPTION

The compositions and methods described herein employ gene therapy using adeno-associated virus (AAV) as a means for treating heritable ocular genetic disorders. More specifically, the methods and compositions described herein employ the use of pre-mRNA trans-splicing as a gene therapy, both ex vivo and in vivo, for the treatment of ocular diseases caused by defects in large genes. In one embodiment, these compositions and methods overcome the problem caused by the packaging limit for nucleic acids into AAV being limited to 4700 nucleotides. When including sequences necessary for producing an effective rAAV therapeutic and expressing the RNA-trans-splicing molecule (RTM), the effective size constraint for the RTM containing the ocular gene sequences is about 4000 nucleotides. These methods and compositions are particularly desirable for treatment of ocular disorders caused by defects in genes exceeding the size necessary for incorporation and expression in an AAV, such as ABCA4, CEP290 and MYO74, among other genes.

### Definitions

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. The definitions used herein are provided for clarity only and are not intended to limit the claimed invention.

As used herein, the term “mammalian subject” or “subject” includes any mammal in need of these methods of treatment or prophylaxis, including particularly humans. Other mammals in need of such treatment or prophylaxis include dogs, cats, or other domesticated animals, horses, livestock, laboratory animals, including non-human primates, etc. The subject may be male or female. In one embodiment, the subject has, or is at risk of developing an ocular disorder. In another embodiment, the subject has shown clinical signs of an ocular disorder, particular a disorder related to a defect or mutation in the genes ABCA4, CEP290, or MYO7A.

The term “ocular disorder” includes, without limitation, Stargardt disease (autosomal dominant or autosomal recessive), retinitis pigmentosa, rod-cone dystrophy, Leber's congenital amaurosis, Usher's syndrome, Bardet-Biedl Syndrome, Best disease, retinoschisis, untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, glaucoma, or retinal vein occlusion. In another embodiment, the subject has, or is at risk of developing glaucoma, Leber's hereditary optic neuropathy, lysosomal storage disorder, or peroxisomal disorder.

Clinical signs of ocular disease include, but are not limited to, decreased peripheral vision, decreased central (reading) vision, decreased night vision, loss of color perception, reduction in visual acuity, decreased photoreceptor function, pigmentary changes. In another embodiment, the subject has been diagnosed with STGD1. In another embodiment, the subject has been diagnosed with a juvenile onset macular degeneration, fundus flavimaculatus. In another embodiment, the subject has been diagnosed with cone-rod dystrophy. In another embodiment, the subject has been diagnosed with retinitis pigmentosa. In another embodiment, the subject has been diagnosed with age-related macular degeneration (AMD). In another embodiment, the subject has been diagnosed with LCA10. In yet another embodiment, the subject has not yet shown clinical signs of these ocular pathologies.

As used herein, the term “treatment” or “treating” is defined as one or more of reducing onset or progression of an ocular disease, preventing disease, reducing the severity of the disease symptoms, or retarding their progression, removing the disease symptoms, delaying onset of disease or monitoring progression of disease or efficacy of therapy in a given subject.

As used herein, the term “selected cells” refers to an ocular cell, which is any cell associated with the function of, the eye. In one embodiment, the ocular cell is a photoreceptor cell. In another embodiment, the term refers to rod, cone and photosensitive ganglion cells, retinal pigment epithelium (RPE) cells, Mueller cells, bipolar cells, horizontal cells, amacrine cells. Some genes are expressed in the eye as well as in other organs. For example, CEP290 is expressed in kidney epithelium and in the central nervous system; MY07A is expressed in cochlear hair cells. “Selected cells” may also include these extra-ocular cells.

As used herein, the term “host cell” may refer to the packaging cell line in which the rAAV is produced from the plasmid. In the alternative, the term “host cell” may refer to the target cell in which expression of the transgene is desired.

An RNA trans-splicing molecule (RTM) has three main elements: (a) an anti-sense binding domain (BD) which is the element that confers specificity by tethering the RTM to its target pre-mRNA; (b) a 3′ and/or 5′ splice site; and (c) a coding sequence to be trans-spliced, which can re-write most of the targeted pre-mRNA by replacing one or numerous exons anywhere in a message.

Codon optimization refers to modifying a nucleic acid sequence to change individual nucleic acids without any resulting change in the encoded amino acid. This process may be performed on any of the sequences described in this specification to enhance expression or stability. Codon optimization may be performed in a manner such as that described in, e.g., U.S. Pat. Nos. 7,561,972; 7,561,973; and 7,888,112, incorporated herein by reference, and conversion of the sequence surrounding the translational start site to a consensus Kozak sequence. See, Kozak et al, Nucleic Acids Res. 15 (20): 8125-8148, incorporated herein by reference.

The term “homologous” refers to the degree of identity between sequences of two nucleic acid sequences. The homology of homologous sequences is determined by comparing two sequences aligned under optimal conditions over the sequences to be compared. The sequences to be compared herein may have an addition or deletion (for example, gap and the like) in the optimum alignment of the two sequences. Such a sequence homology can be calculated by creating an alignment using, for example, the ClustalW algorithm (Nucleic Acid Res., 22(22): 4673 4680 (1994). Commonly available sequence analysis software, more specifically, Vector NTI, GENETYX, BLAST or analysis tools provided by public databases may also be used.

The term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the synthetic is administered. Examples of suitable pharmaceutical carriers are described in “Remington's Pharmaceutical sciences” by E. W. Martin.

The terms “a” or “an” refers to one or more, for example, “a gene” is understood to represent one or more such genes. As such, the terms “a” (or “an”), “one or more,” and “at least one” are used interchangeably herein.

As used herein, the term “about” means a variability of ±0.1 to 10% from the reference given, unless otherwise specified.

With regard to the following description, it is intended that each of the compositions herein described, is useful, in another embodiment, in the methods of treatment described herein. In addition, it is also intended that each of the compositions herein described as useful in the methods, is itself an embodiment. While various embodiments in the specification are presented using “comprising” language, which is inclusive of other components or steps, under other circumstances, a related embodiment is also intended to be interpreted and described using “consisting of” or “consisting essentially of” language, which is exclusive of all or any components or steps which significantly change the embodiment.

### Pre-mRNA Trans-Splicing Methods and Molecules

Within a cell, a pre-mRNA intermediate exists that includes non-coding nucleic acid sequences, i.e., introns, and nucleic acid sequences that encode the amino acids forming the gene product. The introns are interspersed between the exons of a gene in the pre-mRNA, and are ultimately excised from the pre-mRNA molecule, when the exons are joined together by a protein complex known as the spliceosome. Using spliceosome activity, one may introduce an alternative exon via the introduction of a second nucleic acid. Spliceosome mediated RNA trans-splicing (SMaRT) has been described as employing an engineered pre-mRNA trans-splicing molecule (RTM) that binds specifically to target pre-mRNA in the nucleus and triggers trans-splicing in a process mediated by the spliceosome. This methodology is described in, for example, Puttaraju M, et al 1999 Nat Biotechnol., 17:246-252; Gruber C et al, 2013 December, Mol. Oncol. 7(6):1056; Avale ME, 2013 July, Hum. Mol. Genet., 22(13):2603-11; Rindt H et al, 2012 December, Cell Mol. Life Sci., 69(24):4191; US Patent Application Publication Nos. 2006/0246422 and 20130059901, and U.S. Pat. Nos. 6,083,702; 6,013,487; 6,280,978; 7,399,753; and 8,053,232. These documents are incorporated herein by reference.

A pre-RNA trans-splicing molecule (RIM) useful as or in the compositions described herein is a molecule that can replace an exon (or multiple exons) in a targeted ocular gene. The design of the RTM permits replacement of the defective or mutated portion of the pre-mRNA exon(s) with a nucleic acid sequence, i.e., the exon (s) having a normal sequence without the defect or mutation. The “normal” sequence can be a wild-type naturally-occurring sequence or a corrected sequence with some other modification, e.g., codon-modified, that is not disease-causing.

The RTM useful in the compositions and methods herein comprises a binding domain that targets binding of the molecule to a pre-mRNA of a target ocular gene expressed within a mammalian ocular cell; a splicing domain containing motifs necessary for a trans-splicing reaction to occur; and a coding domain from an ocular gene. The coding domain contains a nucleotide sequence from the wild-type or corrected cDNA, usually one or more exons, that are necessary to repair the targeted mutation or defects that cause ocular disease. The RTM in one embodiment contains multiple binding domains. The RIM in one embodiment contains multiple splicing domains. The RTM in one embodiment contains multiple coding domains. In one embodiment, RTMs are designed to replace target sequences located on the 3′ portion of the targeted gene. In one embodiment, RTMs are designed to replace target sequences located on the 5′ portion of the targeted gene. In still other embodiments, RIMs are designed to replace an internal target sequence in the gene. The RTMs function to repair the defective gene in the subject's cell by replacing the defective exon and subsequently removing the defective portion of the target pre-mRNA, leaving a functional gene capable of transcribing a function gene product in the cell. The design and assembly of such RTMs follow the descriptions of this technology set out in the patents and references cited throughout this specification and incorporated herein by reference.

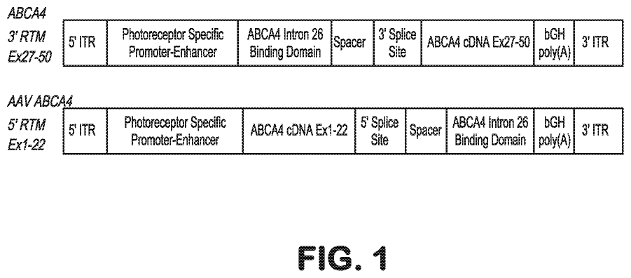

As one example, a 3′ pre-mRNAABCA4 trans-splicing molecule operates as follows: A chimeric mRNA is created through a trans-splicing reaction mediated by the spliceosome between the 5′ splice site of the endogenous target pre-miRNA, ABCA4, and the 3′ splice site of the rAAV-delivered pre-trans-splicing RNA molecule. The RTM molecule binds through specific base pairing to an intron of the endogenous target pre-mRNA and replaces the whole 3′ sequence of the endogenous gene upstream of the targeted intron with the wild type coding sequence of the RTM. The operation of the 5′ and double trans-splicing RTMs can be observed in FIG. 1 of U.S. Pat. No. 8,053,232, incorporated herein by reference.

A 3′ RTM comprises a binding domain which binds to the target pre-mRNA 5′ to the mutation or defect, an optional spacer, a 3′ splice site, and a coding domain that encodes all exons of the ocular target gene that are 3′ to the binding of the binding domain to the target. A 5′ RTM comprising a binding domain binds to the target pre-mRNA 3′ to the mutation or defect, a 5′ splice site, an optional spacer and a coding domain that encodes all exons of the ocular target gene that are 5′ to the binding of the binding domain to the target. A double trans-splicing RTM contains the elements of the 3′ RTM and a second binding domain that targets a sequence of the ocular gene and which binds to the target intro 3′ to the mutation or defect in the target pre-mRNA and a 5′splice site.

For delivery via a recombinant AAV as described herein, in one embodiment, the entire RTM is a nucleic acid sequence of up to 3000 nucleotide bases in length.

### Targeted Ocular Genes

The targeted ocular gene is one that contains one or multiple defects or mutations that cause an ocular disease. In one embodiment described herein, the targeted ocular gene is a mammalian gene with defects known to cause inherited retinal disorders.

The wildtype sequences of the ocular genes and encoded proteins and/or the genomic and chromosomal sequences are available from publically available databases and their accession numbers are provided herein. In addition to these published sequences, all corrections later obtained or naturally occurring conservative and non-disease-causing variants sequences that occur in the human or other mammalian population are also included. Additionally conservative nucleotide replacements or those causing codon optimizations are also included. The sequences as provided by the database accession numbers may also be used to search for homologous sequences in the same or another mammalian organism.

It is anticipated that the target ocular gene nucleic acid sequences and the resulting protein truncates or amino acid fragments identified herein may tolerate certain minor modifications at the nucleic acid level to include, for example, modifications to the nucleotide bases which are silent, e.g., preference codons. In other embodiments, nucleic acid base modifications which change the amino acids, e.g. to improve expression of the resulting peptide/protein are anticipated. Also included as likely modification of fragments are allelic variations, caused by the natural degeneracy of the genetic code.

Also included as modification of the selected ocular genes are analogs, or modified versions, of the encoded protein fragments provided herein. Typically, such analogs differ from the specifically identified proteins by only one to four codon changes. Conservative replacements are those that take place within a family of amino acids that are related in their side chains and chemical properties.

The nucleic acid sequence encoding a normal ocular gene may be derived from any mammal which natively expresses that gene, or homolog thereof. In another embodiment, the ocular gene sequence is derived from the same mammal that the composition is intended to treat. In another embodiment, the ocular gene sequence is derived from a human. In other embodiments, certain modifications are made to the gene sequence in order to enhance the expression in the target cell. Such modifications include codon optimization.

In one embodiment, the gene is ABCA4, which is indicated in the diseases discussed in the background above. The genomic sequence of the DNA for this gene can be found in the NCBI Reference Sequence for Chromosome 1 (135313 bp) at NG_009073.1. The mRNA for the gene as well as the locations of the exons are indicated in the NCBI report. The DNA sequence of ABCA4 provided as NCBI Reference Sequence: NM_000350.2. The amino acid sequence is provided as NCBI Reference Sequence: NP000341.2. TABLE 1 lists mutations in ABCA4 and their locations in certain introns or exons of the nucleotide sequence. TABLE 1 also identifies the associated ocular disease, specific mutation, exon location of mutation, target cells, target intron and it published sequence for designing the binding domain sequence and the exon and its published sequence for use in the coding domain, as well as the 3′ or 5′ direction of the RTM created to contain these components. It should be understood that the binding domain may include sequences complementary to more than target intron sequences, as described below in detail with respect to RTM binding domains. In one embodiment, the RTM is designed to correct ABCA4 mutations p.Leu541Pro and p.Ala1038Val, among others.

In another embodiment, the gene is CEP290. Leber congenital amaurosis comprises a group of early-onset childhood retinal dystrophies characterized by vision loss, nystagmus, and severe retinal dysfunction. Patients usually present at birth with profound vision loss and pendular nystagmus. Electroretinogram (ERG) responses are usually nonrecordable. Other clinical findings may include high hypermetropia, photodysphoria, oculodigital sign, keratoconus, cataracts, and a variable appearance to the fundus. LCA10 is caused by mutation in the CEP290 gene on chromosome 12q21 and may account for as many as 21% of cases of LCA. Mutations in CEP290 can also result in extra-ocular findings, including kidney and CNS abnormalities, and thus can result in syndromes (Senior Loken syndrome. Joubert syndrome, Bardet-Biedl).

The genomic sequence of the DNA for this gene can be found in the NCBI Reference Sequence for Chromosome 12 from nt. 88049013-88142216 (93,204 bp) at NC_000012.12. The mRNA and the exons are identified in NCBI report. The DNA sequence of CEP290 provided as NCBI Reference Sequence: NM_025114.3. The amino acid sequence is provided as NCB1 Reference Sequence: NP0789390.3. The mRNA contains 54 exons and 59 introns (due to alternative splicing). Many mutations of CEP290 and their locations in the nucleotide sequence are known. TABLE 2 lists mutations in CEP290 and their locations in certain introns or exons of the nucleotide sequence. TABLE 2 also identifies the associated ocular disease, specific mutation, exon location of mutation, target cells, the intron and it published sequence for designing the binding domain sequence and the exon and its published sequence for use in the coding domain as well as the 3′ or 5′ direction of the RTM created to contain these components. It should be understood that the binding domain may include sequences complementary to more than target intron sequences, as described below in detail with respect to RTM binding domains. In one embodiment an RTM is designed to correct the exons carry the mutations c2991+1655A to G and Ser1056 to A. In another embodiment, an RTM is designed to target Intron 26 of CEP290.

In another embodiment, the gene is MYO7A. Mutations in this gene are related to Usher Syndrome. Usher syndrome is a condition characterized by hearing loss and progressive vision loss. The loss of vision is caused by an eye disease called retinitis pigmentosa (RP), which affects the layer of light-sensitive retina. Vision loss occurs as the light-sensing cells of the retina gradually deteriorate. Over time, these blind spots enlarge and merge to produce tunnel vision. In some cases of Usher syndrome, vision is further impaired by clouding of the lens of the eye (cataracts). Many people with retinitis pigmentosa retain some central vision throughout their lives, however. The loss of hearing is caused by disease in cochlear hair cells, which also gradually deteriorate. Usher syndrome type I can result from mutations in the CD23, MYO7A, PCDH15, USH1C, or USH1G gene.

More than 250 mutations in the MYO7A gene have been identified in people with Usher syndrome type 1B. Many of these genetic changes alter a single protein building block (amino acid) in critical regions of the myosin VITA protein. Other mutations introduce a premature stop signal in the instructions for the myosin VIIA protein. As a result, an abnormally small version of this protein is made. Some mutations insert or delete small amounts of DNA in the MYO7A gene, which alters the protein. All of these changes cause the production of a nonfunctional myosin VITA protein that adversely affects the development and function of cells in the inner ear and retina, resulting in Usher syndrome.

The genomic sequence of the DNA for this gene can be found in the NCBI Reference Sequence for Chromosome 11 from nt. 77,128,255 to 77,215,240 (86,986 bp) at NC_000011.9. The DNA sequence of MYO7A provided as NCBI Reference Sequence: NM 000260.3. The amino acid sequence is provided as NCBI Reference Sequence: NP 000251.1. The DNA sequence, amino acid sequence, exon sequences and intron sequences are provided for MYO7A online at https://grenada.lumc.nl/LOVD2/Usher_montpellier/refseq/MYO7A_codingDNA.html, last modified Feb. 17, 2010. The mRNA contains 49 exons and 61 introns. Many mutations of MYO7A may be found on the CCHMC Molecular Genetics Laboratory Mutation Database, LOVD v.2.0. See also, TABLE 3 which lists mutations in MYO7A identifying ocular disease, specific mutation, exon location of mutation, target cells, the intron and it published sequence for designing the binding domain sequence and the exon and its published sequence for use in the coding domain as well as the 3′ or 5′ direction of the RTM created to contain these components. It should be understood that the binding domain may include sequences complementary to more than target intron sequences, as described below in detail with respect to RTM binding domains.

### RTM Binding Domains

Each RTM comprises one or more binding domains (BD). In one embodiment, the target binding domain is a nucleic acid sequence, complementary to and in antisense orientation to a sequence of the target pre-mRNA, e.g., ABCA4, to suppress target cis-splicing while enhancing trans-splicing between the RTM and the target. The binding domains generally bind to the target gene 5′ to the mutation or defect in the target pre-mRNA. In one embodiment, the binding domain comprises a part of a sequence complementary to an intron of the targeted gene. In another embodiment, the binding domain comprises a part of a sequence complementary to an exon of the targeted gene. In another embodiment, the binding domain comprises a part of a sequence complementary to an intron of the targeted gene and a part of a sequence complementary to an exon of the targeted gene. In one embodiment the binding domain comprises part of the respective intron upstream of the exon that is primarily functioning as the binding domain. In one embodiment herein, the binding domain is a nucleic acid sequence complementary to the intron closest to the exon sequence that is being corrected. In still another embodiment, the binding domain is targeted to an intron sequence in close proximity to the 3′ or 5′ splice signals of a target intron. In still another embodiment, a binding domain BD sequence can base-pair to the target sequence in two sequences within the target gene, part intron and part exon. The binding domains shown in TABLES 1 to 3 should be understood to encompass any of these regions for a suitable binding domain.

The BD thus binds specifically to the endogenous target pre-mRNA which carries the mutation(s), to anchor the pre-mRNA closely in space to the coding domain of the RTM to permit trans-splicing to occur at the correct position in the target gene. The spliceosome processing machinery of the nucleus then causes successful trans-splicing of the corrected exon for the mutated exon causing the disease.

For use in the RTMs described herein suitable target binding domains may include from 20 up to 50 nucleotides in length. In another embodiment, the target binding domains may include a nucleic acid sequence up to 100 nucleotides in length. In another embodiment, the target binding domains may include a nucleic acid sequence up to 300 nucleotides in length. In another embodiment, the target binding domains may include a nucleic acid sequence up to 500 nucleotides in length. In another embodiment, the target binding domains may include a nucleic acid sequence up to 750 nucleotides in length. In another embodiment, the target binding domains may include a nucleic acid sequence up to 1000 nucleotides in length. In another embodiment, the target binding domains may include a nucleic acid sequence up to 2000 nucleotides or more in length. In certain embodiments, the RTMs contain binding domains that contain sequences on the target pre-mRNA that bind in more than one place. The binding domain may contain any number of nucleotides necessary to stably bind to the target pre-mRNA to permit trans-splicing to occur with the coding domain. In one embodiment, the binding domains are selected using mFOLD structural analysis for accessible loops. Bearing in mind the packaging limitations of the rAAV, the target BD in one embodiment is between about 30 to about 250 nucleotides in length. In one embodiment the binding domains may comprise between and including 70 and 200 nucleotides. In one embodiment the binding domains may comprise between and including 20 and 500 nucleotides. The specificity of the RTM may be increased significantly by increasing the length of the target binding domain. Other lengths may be used depending upon the lengths of the other components of the RTM.

The binding domain may be 100% complementary to the targeted genes' exon, or have sufficient complementarity to be able to hybridize stably with the target pre-mRNA. The degree of complementarity is selected by one of skill in the art based on the need to keep the RTM and the nucleic acid construct containing the necessary sequences for expression and for inclusion in the rAAV within a 3000 or up to 4000 bp limit. The selection of this sequence and strength of hybridization depends on the complementarity and the length of the nucleic acid (See, for example, Sambrook et al., 1989, Molecular Cloning, A Laboratory Manual, 2d Ed., Cold Spring Harbor Laboratory Press, Cold Spring Harbor, N.Y.).

In one embodiment, a suitable RTM binding domain for ABCA4 is a sequence of from 70-200 nucleotides complementary to the target Intron 22 (see Table 1) or to part of the target intron and part of the exon. In another embodiment a suitable RTM binding domain is a sequence from e.g., 70-200 nucleotides complementary to the target Intron 22 or to part of the target intron and part of the exon. Given the teachings herein and TABLE 1, one may select other intron and/or exon targets or portions of introns and their flanking exons to prepare the binding domain based upon the mutation selected and the intron to be targeted. The binding domains of TABLE 1 may be greater than 200 nucleotides in length, as taught herein.

In one embodiment, a suitable RTM binding domain for CEP290 is a sequence of from 70-200 nucleotides complementary to the target Intron 26. Given the teachings herein including TABLE 2, select other intron targets or portions of introns and their flanking exons to prepare the binding domain based upon the mutation selected and the intron to be targeted. The binding domains of TABLE 2 may be greater than 200 nucleotides in length, as taught herein.

In one embodiment, a suitable RTM binding domain for MYO7A is a sequence of from 70-200 nucleotides complementary to the target Intron 32. Given the teachings herein including TABLE 3, select other intron targets or portions of introns and their flanking exons to prepare the binding domain based upon the mutation selected and the intron to be targeted. The binding domains of TABLE 2 may be greater than 200 nucleotides in length, as taught herein.

One of skill in the art may readily select portions of other ocular target genes for correction following the teachings herein.

### RTM Splicing Domains

The splicing domains of the 3′ RTM comprise a strong conserved branch point or branch site (BP) sequence, a polypyrimidine tract (PPT), and a 3′ splice acceptor (AG or YAG) site and/or a 5′ splice donor (GU) site. The splicing domains of the 5′ RTM do not contain the branch point or PPT, but comprise a 5′ splice acceptor/or 3′ splice donor. Splicing domains may be selected by one of skill in the art (see also, the RTM technology documents cited herein).

Briefly, the splicing domain provides essential consensus motifs that are recognized by the spliceosome. The use of BP and PPT follows consensus sequences required for performance of the two phosphoryl transfer reaction involved in cis-splicing and, presumably, also in trans-splicing. In one embodiment a branch point consensus sequence in mammals is YNYURAC (Y=pyrimidine; N=any nucleotide). The underlined A is the site of branch formation. A polypyrimidine tract is located between the branch point and the splice site acceptor and is important for different branch point utilization and 3′ splice site recognition. Consensus sequences for the 5′ splice donor site and the 3′ splice region used in RNA splicing are well known in the art. In addition, modified consensus sequences that maintain the ability to function as 5′ donor splice sites and 3′ splice regions may be used. Briefly, in one embodiment, the 5′ splice site consensus sequence is the nucleic acid sequence AG/GURAGU (where/indicates the splice site). In another embodiment the endogenous splice sites that correspond to the exon proximal to the splice site can be employed to maintain any splicing regulatory signals. In one embodiment, the ABCA4 5′RTM containing as a coding region the sequence encoding exon 1-22 with a binding domain complementary to a region in intron 22 uses the endogenous intron 22 5′ splice site. In another embodiment, the ABCA4 3′RTM encoding exons 27-50 with a binding domain complementary to intron 26 uses the endogenous intron 26 3′ splice site.

In one embodiment a suitable 5′ splice site with spacer is: 5′-GTA AGA GAG CTC GTT GCG ATA TTA T -3′ SEQ ID NO: 5. In one embodiment a suitable 5′ splice site is AGGT.

In one embodiment, a suitable 3′ RTM BP is 5′-TACTAAC-3′. In one embodiment, a suitable 3′ splice site is: 5′-TAC TAA CTG GTA CCT CTT CTT TTT TTT CTG CAG -3 SEQ ID NO: 6 or 5-CAGGT-3′. In one embodiment, a suitable 3′RTM PPT is 5′-TGG TAC CTC TTC TTT TTT TTC TG-3′ SEQ ID NO: 7.

### RTM Target Gene Coding Sequence

The coding domain of the RTMs described herein includes part of the wild type coding sequence to be trans-spliced to the target pre-mRNA. In one embodiment, the coding domain is a single exon of the target gene, which contains the normal wildtype sequence lacking the disease-causing mutations, e.g., Exon 27 of ABCA4. In another embodiment, the coding domain comprises multiple exons which contain multiple mutations causing disease, e.g., Exons 1-22 of ABCA4. Depending upon the location of the exon to be corrected, the RTM may contain multiple exons located at the 5′ or 3′ end of the target gene, or the RTM may be designed to replace an exon in the middle of the gene. For use and delivery in the rAAV, the entire coding sequence of the ocular gene is not useful as the coding domain of RTM, unless this technique is directed to a small ocular gene less than 3000 nucleotides in length. As described herein, to replace an entire large gene, two RTMs, a 3′ and a 5′ RTM can be employed in different rAAV particles.

RTMs described herein can comprise coding domains encoding for one or more exons identified herein and characterized by containing a gene mutation or defect relating to the associated disease, e.g., Exon 27 of ABCA4 may be the coding domain for an RTM designed for the treatment of Stargardt's disease. In TABLEs 1 to 3 herein, the names of the targeted genes and the exons containing likely mutations causing disease are identified.

In one embodiment, the coding domain of a 5′ RTM is designed to replace the exons in the 5′ portion of the targeted gene. In another embodiment, the coding domain of a 3′ RTM is designed to replace the exons in the 3′ portion of a gene. In another embodiment, the coding domain is one or a multiple exons located internally in the gene and the coding domain is located in a double trans-splicing RTMs.

Thus, for example, three possible types of RTMs are useful for treatment of disease caused by defects in e.g., ABCA4: A 5′ trans-splicing RTMs which include a 5′ splice site. After trans-splicing, the 5′ RTM will have changed the 5′ region of the target mRNA; a 3′ RTM which include a 3′ splice site that is used to trans-splice and replace the 3′ region of the target mRNA; and a double trans-splicing RTMs, which carry multiple binding domains along with a 3′ and a 5′ splice site. After trans-splicing, this RTM replaces an internal exon in the processed target mRNA. In other embodiments, the coding domain can include an exon that comprises naturally occurring or artificially introduced stop-codons in order to reduce gene expression; or the RTM can contains other sequences which produce an RNAi-like effect.

For use in treating Stargardt's disease, suitable coding regions of ABCA4 are Exons 1-22 or 27-50, in separate RTMs. For use in treating LCA10, suitable coding regions of CEP290 are Exons 1-26 or exons 27-54 in separate RTMs. For use in treating Usher Syndrome, suitable coding regions of MYO7A are Exons 1-18 or 33-49, in separate RTMs.

Still other coding domains can be constructed by one of skill in the art to replace the entirety of the genes in fragments provided by a 5′ RTM and 3′RTM, and/or a double splicing RTM, given the teachings provided herein.

### Optional Components or Modifications of the RTM

An optional spacer region may be used to separate the splicing domain from the target binding domain in the RTM. The spacer region may be designed to include features such as (i) stop codons which would function to block translation of any unspliced RTM and/or (ii) sequences that enhance trans-splicing to the target pre-mRNA. The spacer may be between 3 to 25 nucleotides or more depending upon the lengths of the other components of the RTM and the rAAV limitations. In one embodiment a suitable 5′ RTM spacer is AGA TCT CGT TGC GAT ATT AT SEQ ID NO: 8. In one embodiment a suitable 3′ spacer is: 5′-GAG AAC ATT ATT ATA GCG TTG CTC GAG -3 SEQ ID NO: 9.

Still other optional components of the RTMs include mini introns, and intronic or exonic enhancers or silencers that would regulate the trans-splicing (See, e.g., the descriptions in the RTM technology publications cited herein.)

In another embodiment, the RTM further comprises at least one safety sequence incorporated into the spacer, binding domain, or elsewhere in the RTM to prevent non-specific trans-splicing. This is a region of the RTM that covers elements of the 3′ and/or 5′ splice site of the RTM by relatively weak complementarity, preventing non-specific trans-splicing. The RTM is designed in such a way that upon hybridization of the binding/targeting portion(s) of the RTM, the 3′ and/or 5′ splice site is uncovered and becomes fully active. Such “safety” sequences comprise a complementary stretch of cis-sequence (or could be a second, separate, strand of nucleic acid) which binds to one or both sides of the RTM branch point, pyrimidine tract, 3′ splice site and/or 5′ splice site (splicing elements), or could bind to parts of the splicing elements themselves. The binding of the “safety” may be disrupted by the binding of the target binding region of the RTM to the target pre-mRNA, thus exposing and activating the RTM splicing elements (making them available to trans-splice into the target pre-mRNA). In another embodiment, the RTM has 3′UTR sequences or ribozyme sequences added to the 3 or 5′ end.

In an embodiment, splicing enhancers such as, for example, sequences referred to as exonic splicing enhancers may also be included in the structure of the synthetic RTMs. Additional features can be added to the RTM molecule, such as polyadenylation signals to modify RNA expression/stability, or 5′ splice sequences to enhance splicing, additional binding regions, “safety”-self complementary regions, additional splice sites, or protective groups to modulate the stability of the molecule and prevent degradation. In addition, stop codons may be included in the RTM structure to prevent translation of unspilced RTMs. Further elements such as a 3′ hairpin structure, circularized RNA, nucleotide base modification, or synthetic analogs can be incorporated into RTMs to promote or facilitate nuclear localization and spliceosomal incorporation, and intra-cellular stability.

The binding of the RTM nucleic acid molecule to the target pre-mRNA is mediated by complementarity (i.e. based on base-pairing characteristics of nucleic acids), triple helix formation or protein-nucleic acid interaction (as described in documents cited herein), In one embodiment, the RTM nucleic acid molecules consist of DNA, RNA or DNA/RNA hybrid molecules, wherein the DNA or RNA is either single or double stranded. Also comprised are RNAs or DNAs, which hybridize to one of the aforementioned RNAs or DNAs preferably under stringent conditions like, for example, hybridization at 60° C. in 2.5×SSC buffer and several washes at 37° C. at a lower buffer concentration like, for example, 0.5×SSC buffer and which encode proteins exhibiting lipid phosphate phosphatase activity and/or association with plasma membranes. When RTMs are synthesized in vitro (synthetic RTMs), such RTMs can be modified at the base moiety, sugar moiety, or phosphate backbone, for example, to improve stability of the molecule, hybridization to the target mRNA, transport into the cell, stability in the cells to enzymatic cleavage, etc. For example, modification of a RTM to reduce the overall charge can enhance the cellular uptake of the molecule. In addition modifications can be made to reduce susceptibility to nuclease or chemical degradation. The nucleic acid molecules may be synthesized in such a way as to be conjugated to another molecule, e.g., a peptide, hybridization triggered cross-linking agent, transport agent, hybridization-triggered cleavage agent, etc.

Various other well-known modifications to the nucleic acid molecules can be introduced as a means of increasing intracellular stability and half-life (see also above for oligonucleotides). Possible modifications are known to the art (see documents cited herein). Modifications, which may be made to the structure of the synthetic RTMs include but are not limited to backbone modifications such as described in the cited RTM technology documents.

### RTAs Useful in Ocular Treatment

Thus, for use in the methods of treating ocular diseases, an RTM comprises a binding domain BD sequence that targets a selected intron of an ocular gene and which binds to the target intron 5′ to the mutation or defect in the target pre-miRNA; an optional spacer; a 3′ splice site; and a target gene coding sequence that encodes an exon of the ocular gene that is 3′ to the binding of the BD to the target. This target gene coding sequence corrects the defects or mutations in the target gene. In another embodiment, the RTM also comprises a second binding domain BD sequence that targets a selected intron of the ocular gene and which binds to the target intron 3′ to the mutation or defect in the target pre-mRNA; and a 5′ splice site for use in replacing an internal exonic sequence. In still another embodiment, the RTM comprises a binding domain BD sequence that targets a selected intron of an ocular gene and which binds to the target intron 3′ to the mutation or defect in the target pre-mRNA; a 5′ splice site; an optional spacer; and a target gene coding sequence that encodes an exon of the ocular gene that is 5′ to the binding of the BD to the target for correcting the defects or mutations in the target gene. In other embodiments, the sequence of the RTM or its components are codon optimized for use in mammalian cells or human cells. In order to fit into the rAAV vector for delivery to the ocular cells, the RTM nucleic acid sequence is less than 4000 kb in length.

As one example, RNA trans-splicing as a treatment of ABCA4-mediated disease, requires constructing and packaging an RTM into AAV. Therefore the RTM is designed to be a nucleic acid molecule of approximately 4,000 nucleic acids in length. As splicing generally occurs between complete exons, in one embodiment, the RTM coding sequence begins at the first nucleotide of the exon following the targeted intron for a 3′ RTM. In another embodiment, the RTM coding sequence ends on the last nucleotide of the exon preceding the targeted intron for a 5′ RTM. Because the spectrum of patients with Stargardt Disease (or in cone-rod dystrophy, autosomal recessive RP, and age-related macular degeneration) have mutations throughout ABCA4, broad correction of as much of the gene as possible is highly desirable.

Thus, in an embodiment described in the Examples below a 3′ RTM and a 5′ RTM are designed to replace exons 1-22 and 27-50 of ABCA4, and thus all of the mutations within those exons. The binding domains employed are sequence complementary to introns 22 and 26, respectively. In still other embodiments, the RTM for ABCA4 may replace only certain exons carrying critical mutations.

An important consideration for the process of designing an RTM is the identification of putative binding domains that are accessible and specific. Larger introns offer more time for an RTM to bind before the spliceosome processes out an intron lariat. By comparison of predicted pre-mRNA folding, candidate binding regions are designed to bind regions in ABCA4 intron 22 and intron 26.

In one embodiment of an RTM, wherein the ocular gene is ACA4, the selected intron is Intron 22 for the 5′ RTM or Intron 26 for the 3′ RTM. In another embodiment, wherein the target ocular gene is CEP290, the selected intron for the 5′ RTM is Intron 26 or for the 3′ RTM is Intron 37. In still another embodiment in which the target gene is MYO7A, the 5′RTM contains a binding sequence complementary to at least a portion of Intron 18 or a 3′RTM contains a binding sequence complementary to at least a portion of Intron 6. Still other suitable RTMs may be designed according to the teachings herein taking into account the mutations and locations provided in TABLEs 1 to 3.

### Recombinant AAV Molecules

A variety of known nucleic acid vectors may be used in these methods to design and assemble the components of the RTM and the recombinant adeno-associated virus (AAV), intended to deliver the RTM to the ocular cells. A wealth of publications known to those of skill in the art discusses the use of a variety of such vectors for delivery of genes (see, e.g., Ausubel et al., Current Protocols in Molecular Biology, John Wiley & Sons, New York, 1989; Kay, M. A. et al, 2001 Nat. Medic., 7(1):33to40; and Walther W. and Stein U., 2000 Drugs, 60(2):249to71). In one embodiment described herein the vector is a recombinant AAV carrying a the RIM and driven by a promoter that expresses RTM in selected ocular cells of the affected subject. Methods for assembly of the recombinant vectors are well-known (see, e.g., International Patent Publication No. WO 00/15822, published Mar. 23, 2000 and other references cited herein).

In certain embodiments described herein, the RTM(s) carrying the ocular gene binding and coding sequences is delivered to the selected cells, e.g., photoreceptor cells, in need of treatment by means of an adeno-associated virus vector. More than 30 naturally occurring serotypes of AAV are available. Many natural variants in the AAV capsid exist, allowing identification and use of an AAV with properties specifically suited for ocular cells. AAV viruses may be engineered by conventional molecular biology techniques, making it possible to optimize these particles for cell specific delivery of the RTM nucleic acid sequences, for minimizing immunogenicity, for tuning stability and particle lifetime, for efficient degradation, for accurate delivery to the nucleus, etc.

The expression of the RTMs described herein can be achieved in the selected cells through delivery by recombinantly engineered AAVs or artificial AAV's that contain sequences encoding the desired RTM. The use of AAVs is a common mode of exogenous delivery of DNA as it is relatively non-toxic, provides efficient gene transfer, and can be easily optimized for specific purposes. Among the serotypes of AAVs isolated from human or non-human primates (NHP) and well characterized, human serotype 2 has been widely used for efficient gene transfer experiments in different target tissues and animal models. Other AAV serotypes include, but are not limited to, AAV1, AAV3, AAV4, AAV5, AAV6, AAV7, AAV8 and AAV9. Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9 or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank, PubMed, or the like.

See, e.g., WO 2005/033321 or WO2014/124282 for a discussion of various AAV serotypes, which is incorporated herein by reference.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/5 a useful pseudotyped vector. In another embodiment, the AAV is AAV2/8.

In one embodiment, the vectors useful in compositions and methods described herein contain, at a minimum, sequences encoding a selected AAV serotype capsid, e.g., an AAV2 capsid, or a fragment thereof. In another embodiment, useful vectors contain, at a minimum, sequences encoding a selected AAV serotype rep protein, e.g., AAV2 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV2 origin. Alternatively, vectors may be used in which the rep sequences are from an AAV serotype which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282,199, which is incorporated by reference herein.

A suitable recombinant adeno-associated virus (AAV) is generated by culturing a host cell which contains a nucleic acid sequence encoding an adeno-associated virus (AAV) serotype capsid protein, or fragment thereof, as defined herein; a functional rep gene; a minigene composed of, at a minimum, AAV inverted terminal repeats (ITRs) and the RTM nucleic acid sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

In one embodiment, the AAV comprises a promoter (or a functional fragment of a promoter). The selection of the promoter to be employed in the rAAV may be made from among a wide number of constitutive or inducible promoters that can express the selected transgene in the desired target cell. See, e.g., the list of promoters identified in International Patent Publication No. WO2014/12482, published Aug. 14, 2014, incorporated by reference herein. In one embodiment, the promoter is “cell specific”. The term “cell-specific” means that the particular promoter selected for the recombinant vector can direct expression of the selected transgene in a particular cell or ocular cell type. In one embodiment, the promoter is specific for expression of the transgene in photoreceptor cells. In another embodiment, the promoter is specific for expression in the rods and/or cones. In another embodiment, the promoter is specific for expression of the transgene in RPE cells. In another embodiment, the promoter is specific for expression of the transgene in ganglion cells. In another embodiment, the promoter is specific for expression of the transgene in Mueller cells. In another embodiment, the promoter is specific for expression of the transgene in bipolar cells. In another embodiment, the transgene is expressed in any of the above noted ocular cells.

In another embodiment, promoter is the native promoter for the target ocular gene to be expressed. Useful promoters include, without limitation, the rod opsin promoter, the red-green opsin promoter, the blue opsin promoter, the cGMP-β-phosphodiesterase promoter, the mouse opsin promoter (Beltran et al 2010 cited above), the rhodopsin promoter (Mussolino et al, Gene Ther, July 2011, 18(7):637-45); the alpha-subunit of cone transducin (Morrissey et al, BMC Dev, Biol, January 2011, 11:3); beta phosphodiesterase (PDE) promoter; the retinitis pigmentosa (RPI) promoter (Nicord et al, J. Gene Med, December 2007, 9(12):1015-23); the NXNL2/NXNL1 promoter (Lambard et al, PLoS One, Oct. 2010, 5(10):e13025), the RPE65 promoter; the retinal degeneration slow/peripherin 2 (Rds/perph2) promoter (Cai et al, Exp Eye Res. 2010 August; 91(2):186-94); and the VMD2 promoter (Kachi et al, Human Gene Therapy, 2009 (20:31-9)). Each of these documents is incorporated by reference herein.

Other conventional regulatory sequences contained in the mini-gene or rAAV are also disclosed in documents such as WO2014/124282 and others cited and incorporated by reference herein. One of skill in the art may make a selection among these, and other, expression control sequences without departing from the scope described herein

The desired AAV minigene is composed of, at a minimum, the RTM described herein and its regulatory sequences, and 5′ and 3′ AAV inverted terminal repeats (ITRs).

In one embodiment, the ITRs of AAV serotype 2 are used. In another embodiment, the ITRs of AAV serotype 5 or 8 are used. However, ITRs from other suitable serotypes may be selected. It is this minigene which is packaged into a capsid protein and delivered to a selected host cell.

The minigene, rep sequences, cap sequences, and helper functions required for producing the rAAV may be delivered to the packaging host cell in the form of any genetic element which transfers the sequences carried thereon. The selected genetic element may be delivered by any suitable method, including those described herein. The methods used to construct any embodiment described herein are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY. Similarly, methods of generating rAAV virions are well known and the selection of a suitable method is not a limitation on the present invention. See, e.g., K. Fisher et al, 1993 J. Virol., 70:520to532 and U.S. Pat. No. 5,478,745, among others. These publications are incorporated by reference herein.

In another aspect, the RTM minigene is prepared in a proviral plasmid, such as those disclosed in International Patent Publication No. WO2012/158757, incorporated herein by reference. Such a proviral plasmid contains a modular recombinant AAV genome comprising in operative association comprising: a wildtype 5′ AAV2 ITR sequence flanked by unique restriction sites that permit ready removal or replacement of said ITR; a promoter comprising a 49 nucleic acid cytomegalovirus sequence upstream of a cytomegalovirus (CMV)-chicken beta actin sequence, or a photoreceptor-specific promoter/enhancer, the promoter flanked by unique restriction sites that permit ready removal or replacement of the entire promoter sequence, and the upstream sequence flanked by unique restriction sites that permit ready removal or replacement of only the upstream CMV or enhancer sequence, from the promoter sequence. The RTM described herein is inserted into the site of a multi-cloning polylinker, wherein the RTM is operatively linked to, and under the regulatory control of, the promoter. A bovine growth hormone polyadenylation sequence flanked by unique restriction sites that permit ready removal or replacement of said polyA sequence; and a wildtype 3′ AAV2 ITR sequence flanked by unique restriction sites that permit ready removal or replacement of the 3′ ITR; are also part of this plasmid. The plasmid backbone comprises the elements necessary for replication in bacterial cells, e.g., a kanamycin resistance gene, and is itself flanked by transcriptional terminator/insulator sequences. As described in the publication immediately referenced, in one embodiment, the plasmid is that designated as p618 comprising the RTM.

In one embodiment, a proviral plasmid comprises (a) a modular recombinant AAV genome comprising in operative association comprising: (i) a wildtype 5′ AAV2 ITR sequence flanked by unique restriction sites that permit ready removal or replacement of said ITR; (ii) a promoter comprising (A) a 49 nucleic acid cytomegalovirus sequence upstream of a cytomegalovirus (CMV)-chicken beta actin sequence, or (B) a photoreceptor-specific promoter/enhancer, or (C) a neuronal cell-specific promoter/enhancer. The promoter is flanked by unique restriction sites that permit ready removal or replacement of the entire promoter sequence, and the upstream sequence flanked by unique restriction sites that permit ready removal or replacement of only the upstream CMV or enhancer sequence, from the promoter sequence. Also part of this proviral plasmid is a multi-cloning polylinker sequence that permits insertion of an RTM sequence including any of those described herein, wherein the RTM is operatively linked to, and under the regulatory control of: the promoter; a bovine growth hormone polyadenylation sequence flanked by unique restriction sites that permit ready removal or replacement of said polyA sequence; and a wildtype 3′ AAV2 ITR sequence flanked by unique restriction sites that permit ready removal or replacement of the 3′ ITR. The proviral plasmid also contains a plasmid backbone comprising the elements necessary for replication in bacterial cells, and further comprising a kanamycin resistance gene, said plasmid backbone flanked by transcriptional terminator/insulator sequences. The proviral plasmid described herein may also contain in the plasmid backbone a non-coding lambda phage 5.1 kb stuffer sequence to increase backbone length and prevent reverse packaging of non-functional AAV genomes.

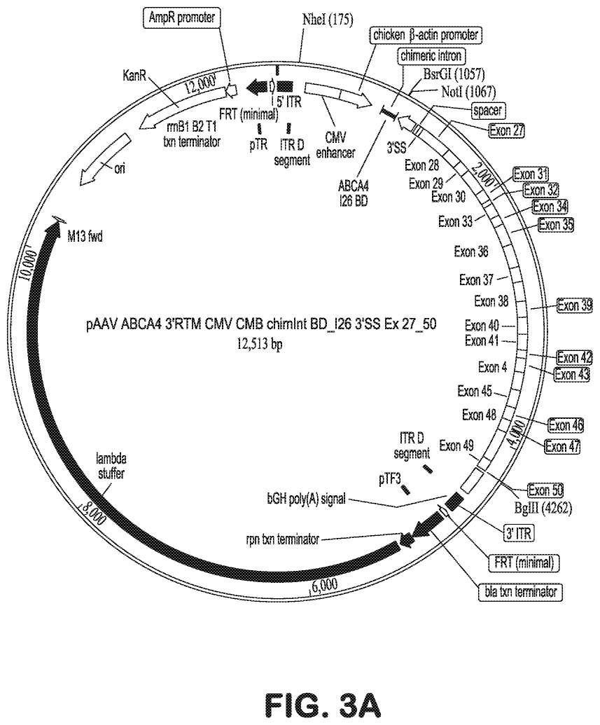

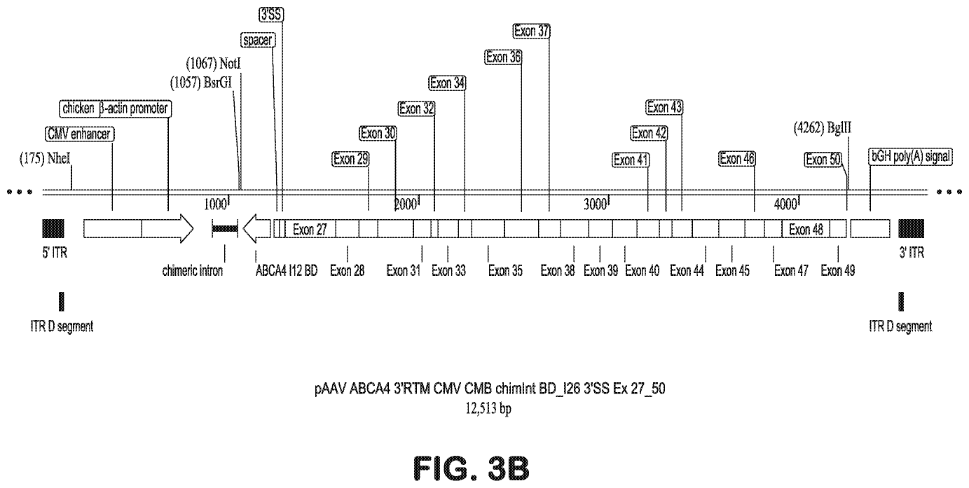

In yet a further aspect, the promoter of the proviral plasmid is modified to reduce the size of the promoter to permit larger RTM sequences to be inserted in the rAAV, In one embodiment, the CMV/CBA hybrid promoter, which normally includes a non-coding exon and intron totaling about 1,000 base pairs, is replaced with a 130 bp chimeric intron (chimera between introns from human β-globin and immunoglobulin heavy chain genes), as illustrated in FIGS. 3A and 3B.

These proviral plasmids are then employed in currently conventional packaging methodologies to generate a recombinant virus expressing the RTM transgene carried by the proviral plasmids. Suitable production cell lines are readily selected by one of skill in the art. For example, a suitable host cell can be selected from any biological organism, including prokaryotic (e.g., bacterial) cells, and eukaryotic cells, including, insect cells, yeast cells and mammalian cells. Briefly, the proviral plasmid is transfected into a selected packaging cell, where it may exist transiently. Alternatively, the minigene or gene expression cassette with its flanking ITRs is stably integrated into the genome of the host cell, either chromosomally or as an episome. Suitable transfection techniques are known and may readily be utilized to deliver the recombinant AAV genome to the host cell. Typically, the proviral plasmids are cultured in the host cells which express the cap and/or rep proteins. In the host cells, the minigene consisting of the RTM with flanking AAV ITRs is rescued and packaged into the capsid protein or envelope protein to form an infectious viral particle. Thus a recombinant AAV infectious particle is produced by culturing a packaging cell carrying the proviral plasmid in the presence of sufficient viral sequences to permit packaging of the gene expression cassette viral genome into an infectious AAV envelope or capsid.

As other aspects of this invention are all of the components of the rAAV particle construction including the cell culture comprising host cells transfected with the proviral plasmid or any similar plasmid and the recombinant AAV infectious particle comprising an RTM as described herein.

TABLES 1, 2 and 3 as referred to above are provided below.

TABLE 1

| Target | | | | | | RTM | | Splice |
|---|---|---|---|---|---|---|---|---|
| Ocular | Ocular | | | | | Binding Domain | | Site |
| Gene | Disease | Mutation | Exon | Target Cells | Intron/SEQ | Seq | Exon/Seq | Seq |
| ABCA4 | STGD | c.2T>C (p.Met1?) | ex1 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.20T>A (p.Ile7Lys) | ex1 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.32T>C (p.Leu11Pro) | ex1 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.38_46del9 | ex1 | Photoreceptors | Int 22 (NG_009073.1) | ~30-250 nts | Ex 1-22 [c.1A p.Met1] | 5' |
| | | (p.Lys13_Trp15del) | | | | complementary | through [c.3328G | |
| | | | | | | to target intron | p.Ser1109+1] | |
| | | | | | | | (NM_000350.2) | |
| ABCA4 | STGD | c.45G>A (p.Trp15*) | ex1 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.52C>T (p.Arg18Trp) | ex1 | Photoreceptors | Int 22 (NG_009073.1) | ~30-250 nts | Ex 1-22 [c.1A p.Met1] | 5' |
| | CRD | | | | | complementary | through [c.3328G | |
| | | | | | | to target intron | p.Ser1109+1] | |
| | | | | | | | (NM_000350.2) | |
| ABCA4 | STGD | c.61C>T (p.Gln21*) | ex1 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.70C>T (p.Arg24Cys) | ex2 | Photoreceptors | Int 22 (NG_009073.1) | ~30-250 nts | Ex 1-22 [c.1A p.Met1] | 5' |
| | CRD | | | | | complementary | through [c.3328G | |
| | | | | | | to target intron | p.Ser1109+1] | |
| | | | | | | | (NM_000350.2) | |
| ABCA4 | STGD | c.71G>A (p.Arg24His) | ex2 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.91T>C (p.Trp31Arg) | ex2 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.108delT (p.Leu37fs) | ex2 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.123G>A (p.Trp41*) | ex2 | Photoreceptors | Int 22 (NG_009073.1) | ~30-250 nts | Ex 1-22 [c.1A p.Met1] | 5 |
| | | | | | | complementary | through [c.3328G | |
| | | | | | | to target intron | p.Ser1109+1] | |
| | | | | | | | (NM_000350.2) | |
| ABCA4 | STGD | c.122G>A (p.Trp41*) | ex2 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.160T>G (p.Cys54Gly) | ex2 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.160+1G>A (−) | int2 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.160+2T>C (−) | int2 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.161−1G>A (−) | int2 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.161G>A (p.Cys54Tyr) | ex3 | Photoreceptors | Int 22 (NG_009073.1) | ~30-250 nts | Ex 1-22 [c.1A p.Met1] | 5' |
| | AMD | | | | | complementary | through [c.3328G | |
| | | | | | | to target intron | p.Ser1109+1] | |
| | | | | | | | (NM_000350.2) | |
| ABCA4 | STGD | c.161G>T (p.Cys54Phe) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.164A>C (p.His55Pro) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.164A>G (p.His55Arg) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.174C>G (p.Asn58Lys) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.178G>A (p.Ala60Thr) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.184C>T (p.Pro62Ser) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.190G>C (p.Ala64Pro) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.194G>A (p.Gly65Glu) | ex3 | Photoreceptors | Int 22 (NG_009073.1) | ~30-250 nts | Ex 1-22 [c.1A p.Met1] | 5' |
| | CRD | | | | | complementary | through [c.3328G | |
| | | | | | | to target intron | p.Ser1109+1] | |
| | | | | | | | (NM_000350.2) | |
| ABCA4 | STGD, | c.203C>T (p.Pro68Leu) | ex3 | Photoreceptors | Int 22 (NG_009073.1) | ~30-250 nts | Ex 1-22 [c.1A p.Met1] | 5' |
| | AMD | | | | | complementary | through [c.3328G | |
| | | | | | | to target intron | p.Ser1109+1] | |
| | | | | | | | (NM_000350.2) | |
| ABCA4 | STGD, | c.203C>G (p.Pro68Arg) | ex3 | Photoreceptors | Int 22 (NG_009073.1) | ~30-250 nts | Ex 1-22 [c.1A p.Met1] | 5' |
| | CRD | | | | | complementary | through [c.3328G | |
| | | | | | | to target intron | p.Ser1109+1] | |
| | | | | | | | (NM_000350.2) | |
| ABCA4 | STGD | c.214G>A (p.Gly72Arg) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.223T>G (p.Cys75Gly) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.230T>A (p.Val77Glu) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.247_250delCAAA | ex3 | Photoreceptors | Int 22 (NG_009073.1) | ~30-250 nts | Ex 1-22 [c.1A p.Met1] | 5' |
| | | (p.Gln83fs) | | | | complementary | through [c.3328G | |
| | | | | | | to target intron | p.Ser1109+1] | |
| | | | | | | | (NM_000350.2) | |
| ABCA4 | CRD, | c.250_251insCAAA | ex3 | Photoreceptors | Int 22 (NG_009073.1) | ~30-250 nts | Ex 1-22 [c.1A p.Met1] | 5' |
| | STGD | (p.Ser84fs) | | | | complementary | through [c.3328G | |
| | | | | | | to target intron | p.Ser1109+1] | |
| | | | | | | | (NM_000350.2) | |
| ABCA4 | STGD | c.286A>G (p.Asn96Asp) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.286A>C (p.Asn96His) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.286A>T (p.Asn96Tyr) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.288C>A (p.Asn96Lys) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.288C>G (p.Asn96Lys) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.296_297insA (p.Asn99fs) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.298T>C (p.Ser100Pro) | ex3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.302+1G>A (−) | int3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.302+4A>C (−) | int3 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.317A>T (p.Tyr106Phe) | ex4 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.318T>G (p.Tyr106*) | ex4 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.319C>T (p.Arg107*) | ex4 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.400C>T (p.Gln134*) | ex4 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.323A>T (p.Asp108Val) | ex4 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.327dupT (p.Gln110fs) | ex4 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.355_356delAG | ex4 | Photoreceptors | Int 22 (NG_009073.1) | ~30-250 nts | Ex 1-22 [c.1A p.Met1] | 5' |
| | | (p.Ser119fs) | | | | complementary | through [c.3328G | |
| | | | | | | to target intron | p.Ser1109+1] | |
| | | | | | | | (NM_000350.2) | |
| ABCA4 | STGD | c.428C>T (p.Pro143Leu) | ex4 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.454C>T (p.Arg152*) | ex5 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.455G>A (p.Arg152Gln) | ex5 | Photoreceptors | Int 22 (NG_009073.1) | ~30-250 nts | Ex 1-22 [c.1A p.Met1] | 5' |
| | FFM | | | | | complementary | through [c.3328G | |
| | | | | | | to target intron | p.Ser1109+1] | |
| | | | | | | | (NM_000350.2) | |
| ABCA4 | STGD | c.466A>G (p.Ile156Val) | ex5 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | CRD | c.481G>A (p.Glu161Lys) | ex5 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.514G>A (p.Gly172Ser) | ex5 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.560G>A (p.Arg187His) | ex5 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.564delA (p.Glu189fs) | ex5 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.570G>C (p.Gln190His) | ex5 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.571−2A>G (−) | int5 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.571−2A>T (−) | int5 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.571−1G>T (−) | int5 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.574G>A (p.Ala192Thr) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.618C>G (p.Ser206Arg) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.618C>A (p.Ser206Arg) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.634C>T (p.Arg212Cys) | ex6 | Photoreceptors | Int 22 (NG_009073.1) | ~30-250 nts | Ex 1-22 [c.1A p.Met1] | 5' |
| | AMD, | | | | | complementary | through [c.3328G | |
| | CRD | | | | | to target intron | p.Ser1109+1] | |
| | | | | | | | (NM_000350.2) | |
| ABCA4 | STGD | c.635G>A (p.Arg212His) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.655A>T (p.Arg219*) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.656G>C (p.Arg219Thr) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.658C>T (p.Arg220Cys) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.664delG (p.Ala222fs) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.666_678del13 | ex6 | Photoreceptors | Int 22 (NG_009073.1) | ~30-250 nts | Ex 1-22 [c.1A p.Met1] | 5' |
| | AMD | (p.Lys223fs) | | | | complementary | through [c.3328G | |
| | | | | | | to target intron | p.Ser1109+1] | |
| | | | | | | | (NM_000350.2) | |
| ABCA4 | STGD | c.667A>C (p.Lys223Gln) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.671delC (p.Thr224fs) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.677G>T (p.Arg226Leu) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.688T>A (p.Cys230Ser) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.700C>T (p.Gln234*) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.730_731delCT | ex6 | Photoreceptors | Int 22 (NG_009073.1) | ~30-250 nts | Ex 1-22 [c.1A p.Met1] | 5' |
| | | (p.Leu244fs) | | | | complementary | through [c.3328G | |
| | | | | | | to target intron | p.Ser1109+1] | |
| | | | | | | | (NM_000350.2) | |
| ABCA4 | STGD | c.731T>C (p.Leu244Pro) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.735T>G (p.Tyr245*) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.736G>A (p.Ala246Thr) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.740A>G (p.Asn247Ser) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.740A>T (p.Asn247Ile) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.746A>G (p.Asp249Gly) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.760T>C (p.Phe254Leu) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.763C>T (p.Arg255Cys) | ex6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.768G>T (p.(=)) | ex6 | Photoreceptors | Int 22 (NG_009073.1) | ~30-250 nts | Ex 1-22 [c.1A p.Met1] | 5' |
| | AMD, | | | | | complementary | through [c.3328G | |
| | CRD | | | | | to target intron | p.Ser1109+1] | |
| | | | | | | | (NM_000350.2) | |
| ABCA4 | AMD | c.769−5T>G (−) | int6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.769−1G>T (−) | int6 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.770T>G (p.Leu257Arg) | ex7 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.832delT (p.Ser278fs) | ex7 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.839T>G (p.Met280Arg) | ex7 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | ARRP | c.859- | int7/ | Photoreceptors | Int 22 (NG_009073.1) | ~30-250 nts | Ex 1-22 [c.1A p.Met1] | 5' |
| | | 45_952delinsTCTGACC (−) | ex8 | | | complementary | through [c.3328G | |
| | | | | | | to target intron | p.Ser1109+1] | |
| | | | | | | | (NM_000350.2) | |
| ABCA4 | STGD | c.868C>T (p.Arg290Trp) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.872C>T (p.Pro291Leu) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.880C>T (p.Gln294*) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.885delC (p.Leu296fs) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.899C>A (p.Thr300Asn) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.926C>G (p.Pro309Arg) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.982G>T (p.Glu328*) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.983A>T (p.Glu328Val) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.997C>T (p.Arg333Trp) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.998G>A (p.Arg333Gln) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1007C>G (p.Ser336Cys) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1015T>G (p.Trp339Gly) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1018T>G (p.Tyr340Asp) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1018T>C (p.Tyr340His) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1029_1030insT | ex8 | Photoreceptors | Int 22 (NG_009073.1) | ~30-250 nts | Ex 1-22 [c.1A p.Met1] | 5' |
| | | (p.Asn344*) | | | | complementary | through [c.3328G | |
| | | | | | | to target intron | p.Ser1109+1] | |
| | | | | | | | (NM_000350.2) | |
| ABCA4 | STGD | c.1025_1038del14 | ex8 | Photoreceptors | Int 22 (NG_009073.1) | ~30-250 nts | Ex 1-22 [c.1A p.Met1] | 5' |
| | | (p.Asp342fs) | | | | complementary | through [c.3328G | |
| | | | | | | to target intron | p.Ser1109+1] | |
| | | | | | | | (NM_000350.2) | |
| ABCA4 | STGD | c.1037A>C (p.Lys346Thr) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | CRD | c.1066A>T (p.Lys356*) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1086T>A (p.Tyr362*) | ex8 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1140T>A (p.Asn380Lys) | ex9 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.1220C>T (p.Ala407Val) | ex9 | Photoreceptors | Int 22 (NG_009073.1) | ~30-250 nts | Ex 1-22 [c.1A p.Met1] | 5' |
| | CRD | | | | | complementary | through [c.3328G | |
| | | | | | | to target intron | p.Ser1109+1] | |
| | | | | | | | (NM_000350.2) | |
| ABCA4 | STGD | c.1222C>T (p.Arg408*) | ex9 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1225delA (p.Arg409fs) | ex9 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1239+1G>C (−) | int9 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1240−2A>G (−) | int9 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1245C>A (p.Asn415Lys) | ex10 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1253T>C (p.Phe418Ser) | ex10 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1268A>G (p.His423Arg) | ex10 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1268A>C (p.His423Pro) | ex10 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.1271T>C (p.Val424Ala) | ex10 | Photoreceptors | Int 22 (NG_009073.1) | ~30-250 nts | Ex 1-22 [c.1A p.Met1] | 5' |
| | ARRP | | | | | complementary | through [c.3328G | |
| | | | | | | to target intron | p.Ser1109+1] | |
| | | | | | | | (NM_000350.2) | |
| ABCA4 | STGD | c.1294G>A (p.Glu432Lys) | ex10 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1317G>A (p.Trp439*) | ex10 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1325T>C (p.Phe442Ser) | ex10 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1335C>G (p.Ser445Arg) | ex10 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1344delG (p.Met448fs) | ex10 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | CRD | IVS10-38t>c | in10 | Photoreceptors | Int 22 (NG_009073.1) | ~30-250 nts | | |
| | | | | | | complementary | | |
| | | | | | | to target intron | | |
| ABCA4 | STGD | c.1357G>T (p.Asp453Tyr) | ex11 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1374delA (p.Thr459fs) | ex11 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1381A>T (p.Lys461*) | ex11 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1390_1391delTT | ex11 | Photoreceptors | Int 22 (NG_009073.1) | ~30-250 nts | Ex 1-22 [c.1A p.Met1] | 5' |
| | | (p.Leu464fs) | | | | complementary | through [c.3328G | |
| | | | | | | to target intron | p.Ser1109+1] | |
| | | | | | | | (NM_000350.2) | |
| ABCA4 | STGD, | c.1411G>A (p.Glu471Lys) | ex11 | Photoreceptors | Int 22 (NG_009073.1) | ~30-250 nts | Ex 1-22 [c.1A p.Met1] | 5' |
| | AMD | | | | | complementary | through [c.3328G | |
| | | | | | | to target intron | p.Ser1109+1] | |
| | | | | | | | (NM_000350.2) | |
| ABCA4 | STGD | c.1494C>A (p.Asp498Glu) | ex11 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1506_1514del9 | ex11 | Photoreceptors | Int 22 (NG_009073.1) | ~30-250 nts | Ex 1-22 [c.1A p.Met1] | 5' |
| | | (p.Phe503_Ile505del) | | | | complementary | through [c.3328G | |
| | | | | | | to target intron | p.Ser1109+1] | |
| | | | | | | | (NM_000350.2) | |
| ABCA4 | STGD | c.1513_1517del5 | ex11 | Photoreceptors | Int 22 (NG_009073.1) | ~30-250 nts | Ex 1-22 [c.1A p.Met1] | 5' |
| | | (p.Ile505*) | | | | complementary | through [c.3328G | |
| | | | | | | to target intron | p.Ser1109+1] | |
| | | | | | | | (NM_000350.2) | |
| ABCA4 | STGD | c.1522C>T (p.Arg508Cys) | ex11 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1531C>T (p.Arg511Cys) | ex11 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1554+1G>A (−) | int11 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1555−1seG>A (−) | int11 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1569T>G (p.Asp523Glu) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1574T>G (p.Phe525Cys) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1574T>C (p.Phe525Ser) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1609C>T (p.Arg537Cys) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1610G>A (p.Arg537His) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1613C>A (p.Ala538Asp) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1613C>T (p.Ala538Val) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.1622T>C (p.Leu541Pro) | ex12 | Photoreceptors | Int 22 (NG_009073.1) | ~30-250 nts | Ex 1-22 [c.1A p.Met1] | 5' |
| | CRD, | | | | | complementary | through [c.3328G | |
| | RP | | | | | to target intron | p.Ser1109+1] | |
| | | | | | | | (NM_000350.2) | |
| ABCA4 | STGD | c.1645G>C (p.Ala549Pro) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.1648G>A (p.Gly550Arg) | ex12 | Photoreceptors | Int 22 (NG_009073.1) | ~30-250 nts | Ex 1-22 [c.1A p.Met1] | 5' |
| | AMD | | | | | complementary | through [c.3328G | |
| | | | | | | to target intron | p.Ser1109+1] | |
| | | | | | | | (NM_000350.2) | |
| ABCA4 | STGD | c.1654G>A (p.Val552Ile) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1659C>G (p.Phe553Leu) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1699G>A (p.Val567Met) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1714C>T (p.Arg572*) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1715G>A (p.Arg572Gln) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1715G>C (p.Arg572Pro) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1745A>G (p.Asn582Ser) | ex12 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | ARRP, | c.1760+2T>G (−) | int12 | Photoreceptors | Int 22 (NG_009073.1) | ~30-250 nts | Ex 1-22 [c.1A p.Met1] | 5' |
| | CRD, | | | | | complementary | through [c.3328G | |
| | STGD | | | | | to target intron | p.Ser1109+1] | |
| | | | | | | | (NM_000350.2) | |
| ABCA4 | CRD | c.1789C>T (p.Pro597Ser) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1798G>T (p.Asp600Tyr) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1804C>T (p.Arg602Trp) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1805G>A (p.Arg602Gln) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1811T>G (p.Ile604Ser) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1817G>C (p.Gly606Ala) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1817G>A (p.Gly606Asp) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1819G>T (p.Gly607Trp) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1819G>A (p.Gly607Arg) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1819G>C (p.Gly607Arg) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1822T>A (p.Phe608Ile) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1822T>C (p.Phe608Leu) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1823T>A (p.Phe608Tyr) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1843G>T (p.Val615Phe) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1846G>A (p.Glu616Lys) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | ARRP, | c.1847delA (p.Glu616fs) | ex13 | Photoreceptors | Int 22 (NG_009073.1) | ~30-250 nts | Ex 1-22 [c.1A p.Met1] | 5' |
| | STGD | | | | | complementary | through [c.3328G | |
| | | | | | | to target intron | p.Ser1109+1] | |
| | | | | | | | (NM_000350.2) | |
| ABCA4 | STGD | c.1848_1857del10 | ex13 | Photoreceptors | Int 22 (NG_009073.1) | ~30-250 nts | Ex 1-22 [c.1A p.Met1] | 5' |
| | | (p.Ile619fs) | | | | complementary | through [c.3328G | |
| | | | | | | to target intron | p.Ser1109+1] | |
| | | | | | | | (NM_000350.2) | |
| ABCA4 | STGD | c.1852G>A (p.Gly618Arg) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | CRD | c.1853G>A (p.Gly618Glu) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1868A>G (p.Gln623Arg) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.1894delA (p.Ile632fs) | ex13 | Photoreceptors | Int 22 (NG_009073.1) | ~30-250 nts | Ex 1-22 [c.1A p.Met1] | 5' |
| | ARRP | | | | | complementary | through [c.3328G | |
| | | | | | | to target intron | p.Ser1109+1] | |
| | | | | | | | (NM_000350.2) | |
| ABCA4 | STGD | c.1903C>A (p.Gln635Lys) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1903C>T (p.Gln635*) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1906C>T (p.Gln636*) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1908G>T (p.Gln636His) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1917C>A (p.Tyr639*) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1917C>T (p.Tyr639(=)) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1922G>C (p.Cys641Ser) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1927G>A (p.Val643Met) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1928T>G (p.Val643Gly) | ex13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.1933G>A (p.Asp645Asn) | ex13 | Photoreceptors | Int 22 (NG_009073.1) | ~30-250 nts | Ex 1-22 [c.1A p.Met1] | 5' |
| | AMD | | | | | complementary | through [c.3328G | |
| | | | | | | to target intron | p.Ser1109+1] | |
| | | | | | | | (NM_000350.2) | |
| ABCA4 | STGD, | c.1937+1G>A (−) | int13 | Photoreceptors | Int 22 (NG_009073.1) | ~30-250 nts | Ex 1-22 [c.1A p.Met1] | 5' |
| | ARRP | | | | | complementary | through [c.3328G | |
| | | | | | | to target intron | p.Ser1109+1] | |
| | | | | | | | (NM_000350.2) | |
| ABCA4 | STGD | c.1937+2T>C (−) | int13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1938−2A>G (−) | int13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1938−1G>A (−) | int13 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1957C>T (p.Arg653Cys) | ex14 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1964T>G (p.Phe655Cys) | ex14 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1977G>A (p.Met659Ile) | ex14 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1982_1983insG | ex14 | Photoreceptors | Int 22 (NG_009073.1) | ~30-250 nts | Ex 1-22 [c.1A p.Met1] | 5' |
| | | (p.Ala662fs) | | | | complementary | through [c.3328G | |
| | | | | | | to target intron | p.Ser1109+1] | |
| | | | | | | | (NM_000350.2) | |
| ABCA4 | STGD | c.1988G>A (p.Trp663*) | ex14 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.1995C>A (p.Tyr665*) | ex14 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2005_2006delAT | ex14 | Photoreceptors | Int 22 (NG_009073.1) | ~30-250 nts | Ex 1-22 [c.1A p.Met1] | 5' |
| | | (p.Met669fs) | | | | complementary | through [c.3328G | |
| | | | | | | to target intron | p.Ser1109+1] | |
| | | | | | | | (NM_000350.2) | |
| ABCA4 | STGD | c.2041C>T (p.Arg681*) | ex14 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2057T>C (p.Leu686Ser) | ex14 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2069G>T (p.Gly690Val) | ex14 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2090G>A (p.Trp697*) | ex14 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2099G>A (p.Trp700*) | ex14 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2147C>T (p.Thr716Met) | ex14 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.2160+1G>C (−) | int14 | Photoreceptors | Int 22 (NG_009073.1) | ~30-250 nts | Ex 1-22 [c.1A p.Met1] | 5' |
| | AMD | | | | | complementary | through [c.3328G | |
| | | | | | | to target intron | p.Ser1109+1] | |
| | | | | | | | (NM_000350.2) | |
| ABCA4 | STGD | c.2243G>A (p.Cys748Tyr) | ex15 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2285C>A (p.Ala762Glu) | ex15 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2291G>A (p.Cys764Tyr) | ex15 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2292delT (p.Cys764*) | ex15 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2294G>A (p.Ser765Asn) | ex15 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2295T>G (p.Ser765Arg) | ex15 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.2300T>A (p.Val767Asp) | ex15 | Photoreceptors | Int 22 (NG_009073.1) | ~30-250 nts | Ex 1-22 [c.1A p.Met1] | 5' |
| | ARRP, | | | | | complementary | through [c.3328G | |
| | CRD | | | | | to target intron | p.Ser1109+1] | |
| | | | | | | | (NM_000350.2) | |
| ABCA4 | STGD | c.2337C>A (p.Cys779*) | ex15 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2382+1G>A (−) | int15 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2385C>G (p.Ser795Arg) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2385_2400del16 | ex16 | Photoreceptors | Int 22 (NG_009073.1) | ~30-250 nts | Ex 1-22 [c.1A p.Met1] | 5' |
| | | (p.Ser795fs) | | | | complementary | through [c.3328G | |
| | | | | | | to target intron | p.Ser1109+1] | |
| | | | | | | | (NM_000350.2) | |
| ABCA4 | STGD | c.2390T>C (p.Leu797Pro) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2401G>A (p.Ala801Thr) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2409_2410delAT | ex16 | Photoreceptors | Int 22 (NG_009073.1) | ~30-250 nts | Ex 1-22 [c.1A p.Met1] | 5' |
| | | (p.Phe804fs) | | | | complementary | through [c.3328G | |
| | | | | | | to target intron | p.Ser1109+1] | |
| | | | | | | | (NM_000350.2) | |
| ABCA4 | STGD, | c.2453G>A (p.Gly818Glu) | ex16 | Photoreceptors | Int 22 (NG_009073.1) | ~30-250 nts | Ex 1-22 [c.1A p.Met1] | 5' |
| | AMD | | | | | complementary | through [c.3328G | |
| | | | | | | to target intron | p.Ser1109+1] | |
| | | | | | | | (NM_000350.2) | |
| ABCA4 | STGD, | c.2461T>A (p.Trp821Arg) | ex16 | Photoreceptors | Int 22 (NG_009073.1) | ~30-250 nts | Ex 1-22 [c.1A p.Met1] | 5' |
| | AMD | | | | | complementary | through [c.3328G | |
| | | | | | | to target intron | p.Ser1109+1] | |
| | | | | | | | (NM_000350.2) | |
| ABCA4 | STGD | c.2471T>C (p.Ile824Thr) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2486C>T (p.Thr829Met) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2519T>G (p.Met840Arg) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2536G>C (p.Asp846His) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2546T>C (p.Val849Ala) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2552G>A (p.Gly851Asp) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2560G>A (p.Ala854Thr) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2564G>A (p.Trp855*) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2565G>A (p.Trp855*) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2570seT>C | ex16 | Photoreceptors | Int 22 (NG_009073.1) | ~30-250 nts | Ex 1-22 [c.1A p.Met1] | 5' |
| | | (p.Leu857Pro) | | | | complementary | through [c.3328G | |
| | | | | | | to target intron | p.Ser1109+1] | |
| | | | | | | | (NM_000350.2) | |
| ABCA4 | STGD | c.2570delT (p.Asp858fs) | ex16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2587+1G>A (−) | int16 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.2588G>C (p.Gly863Ala) | ex17 | Photoreceptors | Int 22 (NG_009073.1) | ~30-250 nts | Ex 1-22 [c.1A p.Met1] | 5 |
| | AMD, | | | | | complementary | through [c.3328G | |
| | CRD, | | | | | to target intron | p.Ser1109+1] | |
| | ARRP | | | | | | (NM_000350.2) | |
| ABCA4 | STGD | c.2609C>T (p.Pro870Leu) | ex17 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2616_2617delCT | ex17 | Photoreceptors | Int 22 (NG_009073.1) | ~30-250 nts | Ex 1-22 [c.1A p.Met1] | 5' |
| | | (p.Phe873fs) | | | | complementary | through [c.3328G | |
| | | | | | | to target intron | p.Ser1109+1] | |
| | | | | | | | (NM_000350.2) | |
| ABCA4 | STGD | c.2617T>C (p.Phe873Leu) | ex17 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2627A>C (p.Gln876Pro) | ex17 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2644G>A (p.Gly882Ser) | ex17 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2690C>T (p.Thr897Ile) | ex18 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2701A>G (p.Thr901Ala) | ex18 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2791G>A (p.Val931Met) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2798A>T (p.Asn933Ile) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2804T>C (p.Val935Ala) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2819C>G (p.Pro940Arg) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2826delC (p.Arg943fs) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.2827C>T (p.Arg943Trp) | ex19 | Photoreceptors | Int 22 (NG_009073.1) | ~30-250 nts | Ex 1-22 [c.1A p.Met1] | 5' |
| | CRD, | | | | | complementary | through [c.3328G | |
| | AMD, | | | | | to target intron | p.Ser1109+1] | |
| | ARRP | | | | | | (NM_000350.2) | |
| ABCA4 | STGD | c.2828G>A (p.Arg943Gln) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2829delG (p.Pro994fs) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2860T>G (p.Tyr954Asp) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2861A>C (p.Tyr954Ser) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2870A>G (p.Gln957Arg) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2876C>T (p.Thr959Ile) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2883delC (p.Leu962fs) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2888delG (p.Gly963fs) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2893A>T (p.Asn965Tyr) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.2894A>G (p.Asn965Ser) | ex19 | Photoreceptors | Int 22 (NG_009073.1) | ~30-250 nts | Ex 1-22 [c.1A p.Met1] | 5' |
| | AMD | | | | | complementary | through [c.3328G | |
| | | | | | | to target intron | p.Ser1109+1] | |
| | | | | | | | (NM_000350.2) | |
| ABCA4 | STGD | c.2906A>G (p.Lys969Arg) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2908A>C (p.Thr970Pro) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2909C>T (p.Thr970Ile) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2912C>A (p.Thr971Asn) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2915C>A (p.Thr972Asn) | ex19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2919−2A>G (−) | int19 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2920T>C (p.Ser974Pro) | ex20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2932G>T (p.Gly978Cys) | ex20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2933G>A (p.Gly978Asp) | ex20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2947A>G (p.Thr983Ala) | ex20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2948C>T (p.Thr983Ile) | ex20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2966T>C (p.Val989Ala) | ex20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2967delT (p.Gly991fs) | ex20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2971G>C (p.Gly991Arg) | ex20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2971G>T (p.Gly991*) | ex20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.2977_2984del8 | ex20 | Photoreceptors | Int 22 (NG_009073.1) | ~30-250 nts | Ex 1-22 [c.1A p.Met1] | 5' |
| | | (p.Asp993fs) | | | | complementary | through [c.3328G | |
| | | | | | | to target intron | p.Ser1109+1] | |
| | | | | | | | (NM_000350.2) | |
| ABCA4 | STGD | c.3041T>G (p.Leu1014Arg) | ex20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3043T>A (p.Phe1015Ile) | ex20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3050+5G>A (−) | int20 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3055A>G (p.Thr1019Ala) | ex21 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3056C>T (p.Thr1019Met) | ex21 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3064G>A (p.Glu1022Lys) | ex21 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.3085C>T (p.Gln1029*) | ex21 | Photoreceptors | Int 22 (NG_009073.1) | ~30-250 nts | Ex 1-22 [c.1A p.Met1] | 5' |
| | CRD | | | | | complementary | through [c.3328G | |
| | | | | | | to target intron | p.Ser1109+1] | |
| | | | | | | | (NM_000350.2) | |
| ABCA4 | STGD | c.3091A>G (p.Lys1031Glu) | ex21 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3106G>A (p.Glu1036Lys) | ex21 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.3113C>T (p.Ala1038Val) | ex21 | Photoreceptors | Int 22 (NG_009073.1) | ~30-250 nts | Ex 1-22 [c.1A p.Met1] | 5' |
| | CRD, | | | | | complementary | through [c.3328G | |
| | ARRP, | | | | | to target intron | p.Ser1109+1] | |
| | AMD | | | | | | (NM_000350.2) | |
| ABCA4 | STGD | c.3148G>A (p.Gly1050Ser) | ex21 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3149G>A | ex21 | Photoreceptors | Int 22 (NG_009073.1) | ~30-250 nts | Ex 1-22 [c.1A p.Met1] | 5' |
| | | (p.Gly1050Asp) | | | | complementary | through [c.3328G | |
| | | | | | | to target intron | p.Ser1109+1] | |
| | | | | | | | (NM_000350.2) | |
| ABCA4 | STGD | c.3163C>T (p.Arg1055Trp) | ex21 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3187T<C (p.Ser1063Pro) | ex21 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3204A>T (p.Arg1068Ser) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3204A>C (p.Arg1068Ser) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3202_3204delAGA | ex22 | Photoreceptors | Int 22 (NG_009073.1) | ~30-250 nts | Ex 1-22 [c.1A p.Met1] | 5' |
| | | (p.Arg1068del) | | | | complementary | through [c.3328G | |
| | | | | | | to target intron | p.Ser1109+1] | |
| | | | | | | | (NM_000350.2) | |
| ABCA4 | STGD | c.3205A>G (p.Lys1069Glu) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3205_3206dupAA | ex22 | Photoreceptors | Int 22 (NG_009073.1) | ~30-250 nts | Ex 1-22 [c.1A p.Met1] | 5' |
| | | (p.Leu1070fs) | | | | complementary | through [c.3328G | |
| | | | | | | to target intron | p.Ser1109+1] | |
| | | | | | | | (NM_000350.2) | |
| ABCA4 | STGD | c.3211_3212insGT | ex22 | Photoreceptors | Int 22 (NG_009073.1) | ~30-250 nts | Ex 1-22 [c.1A p.Met1] | 5' |
| | | (p.Ser1071fs) | | | | complementary | through [c.3328G | |
| | | | | | | to target intron | p.Ser1109+1] | |
| | | | | | | | (NM_000350.2) | |
| ABCA4 | STGD | c.3212C>T (p.Ser1071Leu) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3215T>C (p.Val1072Ala) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3233G>A (p.Gly1078Glu) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3241A>G (p.Lys1081Glu) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.3259G>A (p.Glu1087Lys) | ex22 | Photoreceptors | Int 22 (NG_009073.1) | ~30-250 nts | Ex 1-22 [c.1A p.Met1] | 5' |
| | CRD | | | | | complementary | through [c.3328G | |
| | | | | | | to target intron | p.Ser1109+1] | |
| | | | | | | | (NM_000350.2) | |
| ABCA4 | STGD | c.3261A>C | ex22 | Photoreceptors | Int 22 (NG_009073.1) | ~30-250 nts | Ex 1-22 [c.1A p.Met1] | 5' |
| | | (p.Glu1087Asp) | | | | complementary | through [c.3328G | |
| | | | | | | to target intron | p.Ser1109+1] | |
| | | | | | | | (NM_000350.2) | |
| ABCA4 | STGD | c.3272G>A (p.Gly1091Glu) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3278A>G | ex22 | Photoreceptors | Int 22 (NG_009073.1) | ~30-250 nts | Ex 1-22 [c.1A p.Met1] | 5' |
| | | (p.Asp1093Gly) | | | | complementary | through [c.3328G | |
| | | | | | | to target intron | p.Ser1109+1] | |
| | | | | | | | (NM_000350.2) | |
| ABCA4 | STGD | c.3279C>A | ex22 | Photoreceptors | Int 22 (NG_009073.1) | ~30-250 nts | Ex 1-22 [c.1A p.Met1] | 5' |
| | | (p.Asp1093Glu) | | | | complementary | through [c.3328G | |
| | | | | | | to target intron | p.Ser1109+1] | |
| | | | | | | | (NM_000350.2) | |
| ABCA4 | STGD | c.3289A>T (p.Arg1097*) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3292C>T (p.Arg1098Cys) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3295T>C (p.Ser1099Pro) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3296C>G (p.Ser1099*) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3296C>A (p.Ser1099*) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3303G>A (p.Trp1101*) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3305A>T (p.Asp1102Val) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD, | c.3322C>T (p.Arg1108Cys) | ex22 | Photoreceptors | Int 22 (NG_009073.1) | ~30-250 nts | Ex 1-22 [c.1A p.Met1] | 5' |
| | AMD | | | | | complementary | through [c.3328G | |
| | | | | | | to target intron | p.Ser1109+1] | |
| | | | | | | | (NM_000350.2) | |
| ABCA4 | STGD | c.3323G>T (p.Arg1108Leu) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3323G>A (p.Arg1108His) | ex22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3329−2seA>G (−) | int22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3329−2seA>T (−) | int22 | Photoreceptors | Int 22 (NG_009073.1) | ~30-250 nts | Ex 1-22 [c.1A p.Met1] | 5' |
| | | | | | | complementary | through [c.3328G | |
| | | | | | | to target intron | p.Ser1109+1] | |
| | | | | | | | (NM_000350.2) | |
| ABCA4 | STGD | c.3335C>A (p.Thr1112Asn) | ex23 | Photoreceptors | | ~30-250 nts | Out of Range | |
| | | | | | | complementary | | |
| | | | | | | to target intron | | |
| ABCA4 | STGD | c.3350C>T (p.Thr1117Ile) | ex23 | Photoreceptors | | ~30-250 nts | Out of Range | |
| | | | | | | complementary | | |
| | | | | | | to target intron | | |
| ABCA4 | STGD, | c.3364G>A (p.Glu1122Lys) | ex23 | Photoreceptors | | ~30-250 nts | Out of Range | |
| | CRD | | | | | complementary | | |
| | | | | | | to target intron | | |
| ABCA4 | STGD | c.3366G>C | ex23 | Photoreceptors | | ~30-250 nts | Out of Range | |
| | | (p.Glu1122Asp) | | | | complementary | | |
| | | | | | | to target intron | | |
| ABCA4 | STGD | c.3377T>C (p.Leu1126Pro) | ex23 | Photoreceptors | | ~30-250 nts | Out of Range | |
| | | | | | | complementary | | |
| | | | | | | to target intron | | |
| ABCA4 | STGD | c.3385C>T (p.Arg1129Cys) | ex23 | Photoreceptors | | ~30-250 nts | Out of Range | |
| | | | | | | complementary | | |
| | | | | | | to target intron | | |
| ABCA4 | STGD | c.3385C>G (p.Arg1129Gly) | ex23 | Photoreceptors | | ~30-250 nts | Out of Range | |
| | | | | | | complementary | | |
| | | | | | | to target intron | | |
| ABCA4 | STGD, | c.3386G>T (p.Arg1129Leu) | ex23 | Photoreceptors | | ~30-250 nts | Out of Range | |
| | AMD | | | | | complementary | | |
| | | | | | | to target intron | | |
| ABCA4 | STGD | c.3393delC (p.Ile1132fs) | ex23 | Photoreceptors | | ~30-250 nts | Out of Range | |
| | | | | | | complementary | | |
| | | | | | | to target intron | | |
| ABCA4 | STGD | c.3412seC>T | ex23 | Photoreceptors | | ~30-250 nts | Out of Range | |
| | | (p.Leu1138Phe) | | | | complementary | | |
| | | | | | | to target intron | | |
| ABCA4 | STGD | c.3449_3451delGCT | ex23 | Photoreceptors | | ~30-250 nts | Out of Range | |
| | | (p.Cys1150del) | | | | complementary | | |
| | | | | | | to target intron | | |
| ABCA4 | ARRP | c.3523−28T>C | in23 | Photoreceptors | | ~30-250 nts | Out of Range | |
| | | | | | | complementary | | |
| | | | | | | to target intron | | |
| ABCA4 | STGD | c.3522+5delG (−) | int23 | Photoreceptors | | ~30-250 nts | Out of Range | |
| | | | | | | complementary | | |
| | | | | | | to target intron | | |
| ABCA4 | STGD | c.3523−2A>T (−) | int23 | Photoreceptors | | ~30-250 nts | Out of Range | |
| | | | | | | complementary | | |
| | | | | | | to target intron | | |
| ABCA4 | STGD | c.3523−1G>A (−) | int23 | Photoreceptors | | ~30-250 nts | Out of Range | |
| | | | | | | complementary | | |
| | | | | | | to target intron | | |
| ABCA4 | STGD | c.3531C>A (p.Cys1177*) | ex24 | Photoreceptors | | ~30-250 nts | Out of Range | |
| | | | | | | complementary | | |
| | | | | | | to target intron | | |
| ABCA4 | STGD | c.3539_3554del16 | ex24 | Photoreceptors | | ~30-250 nts | Out of Range | |
| | | (p.Ser1180fs) | | | | complementary | | |
| | | | | | | to target intron | | |
| ABCA4 | STGD | c.3543delT (p.Lys1182fs) | ex24 | Photoreceptors | | ~30-250 nts | Out of Range | |
| | | | | | | complementary | | |
| | | | | | | to target intron | | |
| ABCA4 | STGD | c.3547seG>T | ex24 | Photoreceptors | | ~30-250 nts | Out of Range | |
| | | (p.Gly1183Cys) | | | | complementary | | |
| | | | | | | to target intron | | |
| ABCA4 | STGD | c.3602T>G (p.Leu1201Arg) | ex24 | Photoreceptors | | ~30-250 nts | Out of Range | |
| | | | | | | complementary | | |
| | | | | | | to target intron | | |
| ABCA4 | STGD | c.3607G>A | ex24 | Photoreceptors | | ~30-250 nts | Out of Range | |
| | | (p.Gly1203Arg) | | | | complementary | | |
| | | | | | | to target intron | | |
| ABCA4 | STGD | c.3607+1G>A (−) | int24 | Photoreceptors | | ~30-250 nts | Out of Range | |
| | | | | | | complementary | | |
| | | | | | | to target intron | | |
| ABCA4 | STGD | c.3608G>A (p.Gly1203Glu) | ex25 | Photoreceptors | | ~30-250 nts | Out of Range | |
| | | | | | | complementary | | |
| | | | | | | to target intron | | |
| ABCA4 | STGD | c.3610G>A | ex25 | Photoreceptors | | ~30-250 nts | Out of Range | |
| | | (p.Asp1204Asn) | | | | complementary | | |
| | | | | | | to target intron | | |
| ABCA4 | STGD | c.3626T>C (p.Met1209Thr) | ex25 | Photoreceptors | | ~30-250 nts | Out of Range | |
| | | | | | | complementary | | |
| | | | | | | to target intron | | |
| ABCA4 | STGD | c.3655G>C (p.Ala1219Pro) | ex25 | Photoreceptors | | ~30-250 nts | Out of Range | |
| | | | | | | complementary | | |
| | | | | | | to target intron | | |
| ABCA4 | STGD | c.3703A>G | ex25 | Photoreceptors | | ~30-250 nts | Out of Range | |
| | | (p.Asn1235Asp) | | | | complementary | | |
| | | | | | | to target intron | | |
| ABCA4 | STGD | c.3749T>C (p.Leu1250Pro) | ex25 | Photoreceptors | | ~30-250 nts | Out of Range | |
| | | | | | | complementary | | |
| | | | | | | to target intron | | |
| ABCA4 | STGD | c.3754G>T (p.Glu1252*) | ex25 | Photoreceptors | | ~30-250 nts | Out of Range | |
| | | | | | | complementary | | |
| | | | | | | to target intron | | |
| ABCA4 | STGD | c.3758C>T (p.Thr1253Met) | ex25 | Photoreceptors | | ~30-250 nts | Out of Range | |
| | | | | | | complementary | | |
| | | | | | | to target intron | | |
| ABCA4 | STGD | c.3808G>T (p.Glu1270*) | ex25 | Photoreceptors | | ~30-250 nts | Out of Range | |
| | | | | | | complementary | | |
| | | | | | | to target intron | | |
| ABCA4 | STGD | c.3812A>G (p.Glu1271Gly) | ex25 | Photoreceptors | | ~30-250 nts | Out of Range | |
| | | | | | | complementary | | |
| | | | | | | to target intron | | |
| ABCA4 | STGD | c.3814-2A>G (−) | int25 | Photoreceptors | | ~30-250 nts | Out of Range | |
| | | | | | | complementary | | |
| | | | | | | to target intron | | |
| ABCA4 | STGD | c.3819_3820insT | ex26 | Photoreceptors | | ~30-250 nts | Out of Range | |
| | | (p.Leu1274fs) | | | | complementary | | |
| | | | | | | to target intron | | |
| ABCA4 | STGD | c.3835_3840del6 | ex26 | Photoreceptors | | ~30-250 nts | Out of Range | |
| | | (p.Asp1279_Ser1280del) | | | | complementary | | |
| | | | | | | to target intron | | |
| ABCA4 | STGD | c.3846delA (p.Gly1283fs) | ex26 | Photoreceptors | | ~30-250 nts | Out of Range | |
| | | | | | | complementary | | |
| | | | | | | to target intron | | |
| ABCA4 | CRD | c.3862+1G>A (−) | int26 | Photoreceptors | | ~30-250 nts | Out of Range | |
| | | | | | | complementary | | |
| | | | | | | to target intron | | |
| ABCA4 | STGD | c.3874C>T (p.Gln1292*) | ex27 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.3898C>T (p.Arg1300*) | ex27 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.3899G>A | ex27 | Photoreceptors | Int 26 (NG_009073.1) | ~30-250 nts | Ex 27-50 [c.3863G | 3' |
| | | (p.Arg1300Gln) | | | | complementary | p.Gly1289-2] through | |
| | | | | | | to target intron | [c.6822A p.Stop2274] | |
| | | | | | | | (NM_000350.2) | |
| ABCA4 | STGD | c.3943C>T (p.Gln1315*) | ex27 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.3970delG (p.Ala1324fs) | ex27 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.3994C>T (p.Gln1332*) | ex27 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4034_4035insCA | ex27 | Photoreceptors | Int 26 (NG_009073.1) | ~30-250 nts | Ex 27-50 [c.3863G | 3' |
| | | (p.Gly1347fs) | | | | complementary | p.Gly1289-2] through | |
| | | | | | | to target intron | [c.6822A p.Stop2274] | |
| | | | | | | | (NM_000350.2) | |
| ABCA4 | CRD | c.4035insCA | ex27 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4036_4037delAC | ex27 | Photoreceptors | Int 26 (NG_009073.1) | ~30-250 nts | Ex 27-50 [c.3863G | 3' |
| | | (p.Thr1346fs) | | | | complementary | p.Gly1289-2] through | |
| | | | | | | to target intron | [c.6822A p.Stop2274] | |
| | | | | | | | (NM_000350.2) | |
| ABCA4 | STGD | c.4073T>C (p.Leu1358Pro) | ex27 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4128G>A (p.?) | ex27 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.4139C>T (p.Pro1380Leu) | ex28 | Photoreceptors | Int 26 (NG_009073.1) | ~30-250 nts | Ex 27-50 [c.3863G | 3' |
| | AMD | | | | | complementary | p.Gly1289-2] through | |
| | | | | | | to target intron | [c.6822A p.Stop2274] | |
| | | | | | | | (NM_000350.2) | |
| ABCA4 | STGD | c.4163T>C (p.Leu1388Pro) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4169T>C (p.Leu1390Pro) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4195G>A (p.Glu1399Lys) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4200C>A (p.Tyr1400*) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4203C>A (p.(=)) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4203C>T (p.(=)) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4216C>T (p.His1406Tyr) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4217A>G (p.His1406Arg) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.4222T>C (p.Trp1408Arg) | ex28 | Photoreceptors | Int 26 (NG_009073.1) | ~30-250 nts | Ex 27-50 [c.3863G | 3' |
| | AMD | | | | | complementary | p.Gly1289-2] through | |
| | | | | | | to target intron | [c.6822A p.Stop2274] | |
| | | | | | | | (NM_000350.2) | |
| ABCA4 | STGD | c.4223G>T (p.Trp1408Leu) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4224G>A (p.Trp1408*) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.4232_4233insTATG | ex28 | Photoreceptors | Int 26 (NG_009073.1) | ~30-250 nts | Ex 27-50 [c.3863G | 3' |
| | AMD | (p.Gln1412fs) | | | | complementary | p.Gly1289-2] through | |
| | | | | | | to target intron | [c.6822A p.Stop2274] | |
| | | | | | | | (NM_000350.2) | |
| ABCA4 | STGD | c.4234C>T (p.Gln1412*) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4248C>A (p.Phe1416Leu) | ex28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4253+4C>T (−) | int28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4253+5G>T (−) | int28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4253+5G>A (−) | int28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4254−15_4261del23 (−) | int28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4254−2A>G (−) | int28 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | AMD | c.4283C>T (p.Thr1428Met) | ex29 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4286T>C (p.Val1429Ala) | ex29 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4289T>C (p.Leu1430Pro) | ex29 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | CRD | c.4297G>A (p.Val1433Ile) | ex29 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4316G>A | ex29 | Photoreceptors | Int 26 (NG_009073.1) | ~30-250 nts | Ex 27-50 [c.3863G | 3' |
| | | (p.Gly1439Asp) | | | | complementary | p.Gly1289-2] through | |
| | | | | | | to target intron | [c.6822A p.Stop2274] | |
| | | | | | | | (NM_000350.2) | |
| ABCA4 | STGD | c.4318T>G (p.Phe1440Val) | ex29 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4319T>C (p.Phe1440Ser) | ex29 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4326C>A | ex29 | Photoreceptors | Int 26 (NG_009073.1) | ~30-250 nts | Ex 27-50 [c.3863G | 3' |
| | | (p.Asn1442Lys) | | | | complementary | p.Gly1289-2] through | |
| | | | | | | to target intron | [c.6822A p.Stop2274] | |
| | | | | | | | (NM_000350.2) | |
| ABCA4 | STGD | c.4328G>A (p.Arg1443His) | ex29 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4342G>A | ex29 | Photoreceptors | Int 26 (NG_009073.1) | ~30-250 nts | Ex 27-50 [c.3863G | 3' |
| | | (p.Gly1448Arg) | | | | complementary | p.Gly1289-2] through | |
| | | | | | | to target intron | [c.6822A p.Stop2274] | |
| | | | | | | | (NM_000350.2) | |
| ABCA4 | STGD | c.4342G>C (p.Gly1448Arg) | ex29 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4346G>A (p.Trp1449*) | ex29 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4352+1G>A (−) | int29 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4353−1G>T (−) | int29 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4363T>C (p.Cys1455Arg) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4417C>A | ex30 | Photoreceptors | Int 26 (NG_009073.1) | ~30-250 nts | Ex 27-50 [c.3863G | 3' |
| | | (p.Leu1473Met) | | | | complementary | p.Gly1289-2] through | |
| | | | | | | to target intron | [c.6822A p.Stop2274] | |
| | | | | | | | (NM_000350.2) | |
| ABCA4 | STGD | c.4436G>A (p.Trp1479*) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4437G>A (p.Trp1479*) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4450C>T (p.Pro1484Ser) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4457C>T (p.Pro1486Leu) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.4462T>C (p.Cys1488Arg) | ex30 | Photoreceptors | Int 26 (NG_009073.1) | ~30-250 nts | Ex 27-50 [c.3863G | 3' |
| | AMD | | | | | complementary | p.Gly1289-2] through | |
| | | | | | | to target intron | [c.6822A p.Stop2274] | |
| | | | | | | | (NM_000350.2) | |
| ABCA4 | STGD | c.4463G>A (p.Cys1488Tyr) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4463G>T (p.Cys1488Phe) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | CRD | c.4469G>A (p.Cys1490Tyr) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4506C>T (p.(=)) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4506C>A (p.Cys1502*) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4517C>T (p.Ala1506Val) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4522G>T (p.Gly1508Cys) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4535C>G (p.Pro1512Arg) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4535C>T (p.Pro1512Leu) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4537delC (p.Gln1513fs) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4537_4538insC | ex30 | Photoreceptors | Int 26 (NG_009073.1) | ~30-250 nts | Ex 27-50 [c.3863G | 3' |
| | | (p.Gln1513fs) | | | | complementary | p.Gly1289-2] through | |
| | | | | | | to target intron | [c.6822A p.Stop2274] | |
| | | | | | | | (NM_000350.2) | |
| ABCA4 | STGD | c.4538A>G | ex30 | Photoreceptors | Int 26 (NG_009073.1) | ~30-250 nts | Ex 27-50 [c.3863G | 3' |
| | | (p.Gln1513Arg) | | | | complementary | p.Gly1289-2] through | |
| | | | | | | to target intron | [c.6822A p.Stop2274] | |
| | | | | | | | (NM_000350.2) | |
| ABCA4 | CRD | c.4538A>C (p.Gln1513Pro) | ex30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | CRD, | c.4539+1G>T (−) | int30 | Photoreceptors | Int 26 (NG_009073.1) | ~30-250 nts | Ex 27-50 [c.3863G | 3' |
| | ARRP, | | | | | complementary | p.Gly1289-2] through | |
| | STGD, | | | | | to target intron | [c.6822A p.Stop2274] | |
| | AMD | | | | | | (NM_000350.2) | |
| ABCA4 | STGD | c.4539+3seA>G (−) | int30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4540−2A>G (−) | int30 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | AMD | c.4549C>A (p.Arg1517Ser) | ex31 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4574T>C (p.Leu1525Pro) | ex31 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.4577C>T (p.Thr1526Met) | ex31 | Photoreceptors | Int 26 (NG_009073.1) | ~30-250 nts | Ex 27-50 [c.3863G | 3' |
| | AMD | | | | | complementary | p.Gly1289-2] through | |
| | | | | | | to target intron | [c.6822A p.Stop2274] | |
| | | | | | | | (NM_000350.2) | |
| ABCA4 | STGD, | c.4594G>A | ex31 | Photoreceptors | Int 26 (NG_009073.1) | ~30-250 nts | Ex 27-50 [c.3863G | 3' |
| | AMD | (p.Asp1532Asn) | | | | complementary | p.Gly1289-2] through | |
| | | | | | | to target intron | [c.6822A p.Stop2274] | |
| | | | | | | | (NM_000350.2) | |
| ABCA4 | STGD | c.4610C>T (p.Thr1537Met) | ex31 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4635−1G>T (−) | int31 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4639A>T (p.Lys1547*) | ex32 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4670A>G (p.Tyr1557Cys) | ex33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4667+1G>A (−) | int32 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4667+2T>C (−) | int32 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | CRD, | c.4685T>C (p.Ile1562Thr) | ex33 | Photoreceptors | Int 26 (NG_009073.1) | ~30-250 nts | Ex 27-50 [c.3863G | 3' |
| | STGD, | | | | | complementary | p.Gly1289-2] through | |
| | AMD | | | | | to target intron | [c.6822A p.Stop2274] | |
| | | | | | | | (NM_000350.2) | |
| ABCA4 | STGD | c.4710delC (p.Ile1571fs) | ex33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | AMD | c.4715C>T (p.Thr1572Met) | ex33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4720G>T (p.Glu1574*) | ex33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4720delG (p.Glu1574fs) | ex33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | AMD | c.4732G>A | ex33 | Photoreceptors | Int 26 (NG_009073.1) | ~30-250 nts | Ex 27-50 [c.3863G | 3' |
| | | (p.Gly1578Arg) | | | | complementary | p.Gly1289-2] through | |
| | | | | | | to target intron | [c.6822A p.Stop2274] | |
| | | | | | | | (NM_000350.2) | |
| ABCA4 | STGD | c.4734_4737del4 | ex33 | Photoreceptors | Int 26 (NG_009073.1) | ~30-250 nts | Ex 27-50 [c.3863G | 3' |
| | | (p.Phe1579*) | | | | complementary | p.Gly1289-2] through | |
| | | | | | | to target intron | [c.6822A p.Stop2274] | |
| | | | | | | | (NM_000350.2) | |
| ABCA4 | STGD | c.4734delG (p.Leu1580*) | ex33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4735_4739delinsCC | ex33 | Photoreceptors | Int 26 (NG_009073.1) | ~30-250 nts | Ex 27-50 [c.3863G | 3' |
| | | (p.Phe1579_Leu1580delins | | | | complementary | p.Gly1289-2] through | |
| | | Pro) | | | | to target intron | [c.6822A p.Stop2274] | |
| | | | | | | | (NM_000350.2) | |
| ABCA4 | STGD | c.4739T>C (p.Leu1580Ser) | ex33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4739delT (p.Leu1580*) | ex33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4748T>C (p.Leu1583Pro) | ex33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4771G>A | ex33 | Photoreceptors | Int 26 (NG_009073.1) | ~30-250 nts | Ex 27-50 [c.3863G | 3' |
| | | (p.Gly1591Arg) | | | | complementary | p.Gly1289-2] through | |
| | | | | | | to target intron | [c.6822A p.Stop2274] | |
| | | | | | | | (NM_000350.2) | |
| ABCA4 | STGD | c.4771G>C (p.Gly1591Arg) | ex33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4773+1G>A (−) | int33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4773+1G>T (−) | int33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4773+2T>C (−) | int33 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.4773+48C>T (−) | int33 | Photoreceptors | Int 26 (NG_009073.1) | ~30-250 nts | Ex 27-50 [c.3863G | 3' |
| | CRD, | | | | | complementary | p.Gly1289-2] through | |
| | ARRP, | | | | | to target intron | [c.6822A p.Stop2274] | |
| | AMD | | | | | | (NM_000350.2) | |
| ABCA4 | STGD | c.4774−2A>C (−) | int33 | \|Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.4793C>A (p.Ala1598Asp) | ex34 | Photoreceptors | Int 26 (NG_009073.1) | ~30-250 nts | Ex 27-50 [c.3863G | 3' |
| | AMD | | | | | complementary | p.Gly1289-2] through | |
| | | | | | | to target intron | [c.6822A p.Stop2274] | |
| | | | | | | | (NM_000350.2) | |
| ABCA4 | STGD | c.4838delA (p.Asp1613fs) | ex34 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4848+1seG>A (−) | int34 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4848+2T>C (−) | int34 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4848+2T>A (−) | int34 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4849G>A (p.?) | ex35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4854G>A (p.Trp1618*) | ex35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4859_4864delinsTCCT | ex35 | Photoreceptors | Int 26 (NG_009073.1) | ~30-250 nts | Ex 27-50 [c.3863G | 3' |
| | | (p.Asn1620fs) | | | | complementary | p.Gly1289-2] through | |
| | | | | | | to target intron | [c.6822A p.Stop2274] | |
| | | | | | | | (NM_000350.2) | |
| ABCA4 | STGD | c.4867G>A (p.Gly1623Ser) | ex35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4875T>A (p.His1625Gln) | ex35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4892T>C (p.Leu1631Pro) | ex35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4909G>A (p.Ala1637Thr) | ex35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | AMD, | c.4918C>T (p.Arg1640Trp) | ex35 | Photoreceptors | Int 26 (NG_009073.1) | ~30-250 nts | Ex 27-50 [c.3863G | 3' |
| | CRD | | | | | complementary | p.Gly1289-2] through | |
| | | | | | | to target intron | [c.6822A p.Stop2274] | |
| | | | | | | | (NM_000350.2) | |
| ABCA4 | STGD, | c.4919G>A | ex35 | Photoreceptors | Int 26 (NG_009073.1) | ~30-250 nts | Ex 27-50 [c.3863G | 3' |
| | AMD | (p.Arg1640Gln) | | | | complementary | p.Gly1289-2] through | |
| | | | | | | to target intron | [c.6822A p.Stop2274] | |
| | | | | | | | (NM_000350.2) | |
| ABCA4 | STGD | c.4926C>G (p.Ser1642Arg) | ex35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.4947delC (p.Glu1650fs) | ex35 | Photoreceptors | Int 26 (NG_009073.1) | ~30-250 nts | Ex 27-50 [c.3863G | 3' |
| | AMD | | | | | complementary | p.Gly1289-2] through | |
| | | | | | | to target intron | [c.6822A p.Stop2274] | |
| | | | | | | | (NM_000350.2) | |
| ABCA4 | STGD | c.4954T>G (p.Tyr1652Asp) | ex35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4956T>G (p.Tyr1652*) | ex35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.4999C>A (p.Gln1667Lys) | ex35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5018+2T>C (−) | intr35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5018+2T>A (−) | intr35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | \|CRD | c.5019−2_5019−1del (−) | intr35 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5041_5055del15 | ex36 | Photoreceptors | Int 26 (NG_009073.1) | ~30-250 nts | Ex 27-50 [c.3863G | 3' |
| | | (p.Val1681_Cys1685del) | | | | complementary | p.Gly1289-2] through | |
| | | | | | | to target intron | [c.6822A p.Stop2274] | |
| | | | | | | | (NM_000350.2) | |
| ABCA4 | STGD | c.5056G>A | ex36 | Photoreceptors | Int 26 (NG_009073.1) | ~30-250 nts | Ex 27-50 [c.3863G | 3' |
| | | (p.Val1686Met) | | | | complementary | p.Gly1289-2] through | |
| | | | | | | to target intron | [c.6822A p.Stop2274] | |
| | | | | | | | (NM_000350.2) | |
| ABCA4 | STGD | c.5059A>T (p.Ile1687Phe) | ex36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5065T>C (p.Ser1689Pro) | ex36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5077G>A (p.Val1693Ile) | ex36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5087G>A (p.Ser1696Asn) | ex36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5107C>G (p.Gln1703Glu) | ex36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5110delG (p.Glu1704fs) | ex36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5113C>T (p.Arg1705Trp) | ex36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5114G>T (p.Arg1705Leu) | ex36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5114G>A | ex36 | Photoreceptors | Int 26 (NG_009073.1) | ~30-250 nts | Ex 27-50 [c.3863G | 3' |
| | | (p.Arg1705Gln) | | | | complementary | p.Gly1289-2] through | |
| | | | | | | to target intron | [c.6822A p.Stop2274] | |
| | | | | | | | (NM_000350.2) | |
| ABCA4 | STGD | c.5138A>G | ex36 | Photoreceptors | Int 26 (NG_009073.1) | ~30-250 nts | Ex 27-50 [c.3863G | 3' |
| | | (p.Gln1713Arg) | | | | complementary | p.Gly1289-2] through | |
| | | | | | | to target intron | [c.6822A p.Stop2274] | |
| | | | | | | | (NM_000350.2) | |
| ABCA4 | STGD | c.5161_5162delAC | ex36 | Photoreceptors | Int 26 (NG_009073.1) | ~30-250 nts | Ex 27-50 [c.3863G | 3' |
| | | (p.Thr1721fs) | | | | complementary | p.Gly1289-2] through | |
| | | | | | | to target intron | [c.6822A p.Stop2274] | |
| | | | | | | | (NM_000350.2) | |
| ABCA4 | STGD | c.5172G>T (p.Trp1724Cys) | ex36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5177C>A (p.Thr1726Asn) | ex36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5186T>C (p.Leu1729Pro) | ex36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | AMD, | c.5196+1G>A (−) | int36 | Photoreceptors | Int 26 (NG_009073.1) | ~30-250 nts | Ex 27-50 [c.3863G | 3' |
| | STGD, | | | | | complementary | p.Gly1289-2] through | |
| | CRD | | | | | to target intron | [c.6822A p.Stop2274] | |
| | | | | | | | (NM_000350.2) | |
| ABCA4 | AMD, | c.5196+2T>C (−) | int36 | Photoreceptors | Int 26 (NG_009073.1) | ~30-250 nts | Ex 27-50 [c.3863G | 3' |
| | STGD, | | | | | complementary | p.Gly1289-2] through | |
| | CRD | | | | | to target intron | [c.6822A p.Stop2274] | |
| | | | | | | | (NM_000350.2) | |
| ABCA4 | STGD | c.5196+2T>G (−) | int36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5196+1_5196+4del4 (−) | int36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5196+1_5196+6del6 (−) | int36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | CRD | c.5197−3seG>A (−) | int36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5197−3seG>C (−) | int36 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5206T>C (p.Ser1736Pro) | ex37 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5222_5232del11 | ex37 | Photoreceptors | Int 26 (NG_009073.1) | ~30-250 nts | Ex 27-50 [c.3863G | 3' |
| | | (p.Leu1741fs) | | | | complementary | p.Gly1289-2] through | |
| | | | | | | to target intron | [c.6822A p.Stop2274] | |
| | | | | | | | (NM_000350.2) | |
| ABCA4 | STGD | c.5242G>A | ex37 | Photoreceptors | Int 26 (NG_009073.1) | ~30-250 nts | Ex 27-50 [c.3863G | 3' |
| | | (p.Gly1748Arg) | | | | complementary | p.Gly1289-2] through | |
| | | | | | | to target intron | [c.6822A p.Stop2274] | |
| | | | | | | | (NM_000350.2) | |
| ABCA4 | STGD | c.5248C>T (p.Gln1750*) | ex37 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5281_5289del9 | ex37 | Photoreceptors | Int 26 (NG_009073.1) | ~30-250 nts | Ex 27-50 [c.3863G | 3' |
| | | (p.Pro1761_Leu1763del) | | | | complementary | p.Gly1289-2] through | |
| | | | | | | to target intron | [c.6822A p.Stop2274] | |
| | | | | | | | (NM_000350.2) | |
| ABCA4 | STGD | c.5285C>A (p.Ala1762Asp) | ex37 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5288T>C (p.Leu1763Pro) | ex37 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5288delT (p.Val1764fs) | ex37 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5300T>C (p.Leu1767Pro) | ex37 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5312+1G>A (−) | int37 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5312+3A>T (−) | int37 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5316G>A (p.Trp1772*) | ex38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5317insA (p.Ala1773fs) | ex38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5318C>A (p.Ala1773Glu) | ex38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5318C>T (p.Ala1773Val) | ex38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5327C>T (p.Pro1776Leu) | ex38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5337C>G (p.Tyr1779*) | ex38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5337C>A (p.Tyr1779*) | ex38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5338C>G (p.Pro1780Ala) | ex38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | CRD | c.5381C>A (p.Ala1794Asp) | ex38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5395A>G | ex38 | Photoreceptors | Int 26 (NG_009073.1) | ~30-250 nts | Ex 27-50 [c.3863G | 3' |
| | | (p.Asn1799Asp) | | | | complementary | p.Gly1289-2] through | |
| | | | | | | to target intron | [c.6822A p.Stop2274] | |
| | | | | | | | (NM_000350.2) | |
| ABCA4 | STGD | c.5413A>G | ex38 | Photoreceptors | Int 26 (NG_009073.1) | ~30-250 nts | Ex 27-50 [c.3863G | 3' |
| | | (p.Asn1805Asp) | | | | complementary | p.Gly1289-2] through | |
| | | | | | | to target intron | [c.6822A p.Stop2274] | |
| | | | | | | | (NM_000350.2) | |
| ABCA4 | STGD, | c.5451G>T (p.Glu1817Asp) | ex38 | Photoreceptors | Int 26 (NG_009073.1) | ~30-250 nts | Ex 27-50 [c.3863G | 3' |
| | AMD | | | | | complementary | p.Gly1289-2] through | |
| | | | | | | to target intron | [c.6822A p.Stop2274] | |
| | | | | | | | (NM_000350.2) | |
| ABCA4 | STGD | c.5459G>C (p.Arg1820Pro) | ex38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5460+1G>A (−) | int38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5460+5G>A (−) | int38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | CRD | c.5461−10T>C (−) | int38 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5512C>T (p.His1838Tyr) | ex39 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5512C>G (p.His1838Asp) | ex39 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5527C>T (p.Arg1843Trp) | ex39 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5537T>C (p.Ile1846Thr) | ex39 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5578C>T (p.Arg1860Trp) | ex39 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | Splice site (c.5584+5G>A) | int39 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5584+6T>C (−) | int39 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | ARRP | c.5584+−70 C>T | int39 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.5585-1G>A (−) | int39 | Photoreceptors | Int 26 (NG_009073.1) | ~30-250 nts | Ex 27-50 [c.3863G | 3' |
| | AMD | | | | | complementary | p.Gly1289-2] through | |
| | | | | | | to target intron | [c.6822A p.Stop2274] | |
| | | | | | | | (NM_000350.2) | |
| ABCA4 | STGD | c.5593C>T (p.His1865Tyr) | ex40 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5603A>T (p.Asn1868Ile) | ex40 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5629_5643dup | ex40 | Photoreceptors | Int 26 (NG_009073.1) | ~30-250 nts | Ex 27-50 [c.3863G | 3' |
| | | (Lys1877_Ala1881dup) | | | | complementary | p.Gly1289-2] through | |
| | | | | | | to target intron | [c.6822A p.Stop2274] | |
| | | | | | | | (NM_000350.2) | |
| ABCA4 | STGD | c.5644A>G | ex40 | Photoreceptors | Int 26 (NG_009073.1) | ~30-250 nts | Ex 27-50 [c.3863G | 3' |
| | | (p.Met1882Val) | | | | complementary | p.Gly1289-2] through | |
| | | | | | | to target intron | [c.6822A p.Stop2274] | |
| | | | | | | | (NM_000350.2) | |
| ABCA4 | STGD | c.5646G>A (p.Met1882Ile) | ex40 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5651T>A (p.Val1884Glu) | ex40 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5653G>A (p.Glu1885Lys) | ex40 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5657G>A (p.Gly1886Glu) | ex40 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5668_5670del | ex40 | Photoreceptors | Int 26 (NG_009073.1) | ~30-250 nts | Ex 27-50 [c.3863G | 3' |
| | | (p.Phe1890del) | | | | complementary | p.Gly1289-2] through | |
| | | | | | | to target intron | [c.6822A p.Stop2274] | |
| | | | | | | | (NM_000350.2) | |
| ABCA4 | STGD | c.5682G>C (p.(=)) | ex40 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5687T>A (p.Val1896Asp) | ex40 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | AMD | c.5693G>A (p.Arg1898His) | ex40 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.5714+5G>A (−) | int40 | Photoreceptors | Int 26 (NG_009073.1) | ~30-250 nts | Ex 27-50 [c.3863G | 3' |
| | AMD, | | | | | complementary | p.Gly1289-2] through | |
| | CRD | | | | | to target intron | [c.6822A p.Stop2274] | |
| | | | | | | | (NM_000350.2) | |
| ABCA4 | STGD | c.5715−2delA (−) | int40 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5761G>A | ex41 | Photoreceptors | Int 26 (NG_009073.1) | ~30-250 nts | Ex 27-50 [c.3863G | 3' |
| | | (p.Val1921Met) | | | | complementary | p.Gly1289-2] through | |
| | | | | | | to target intron | [c.6822A p.Stop2274] | |
| | | | | | | | (NM_000350.2) | |
| ABCA4 | STGD | c.5761G>T (p.Val1921Leu) | ex41 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5762_5763dup | ex41 | Photoreceptors | Int 26 (NG_009073.1) | ~30-250 nts | Ex 27-50 [c.3863G | 3' |
| | | (p.Ala1922fs) | | | | complementary | p.Gly1289-2] through | |
| | | | | | | to target intron | [c.6822A p.Stop2274] | |
| | | | | | | | (NM_000350.2) | |
| ABCA4 | STGD | c.5810T>C (p.Ile1937Thr) | ex41 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5814A>G (p.(=)) | ex41 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5819T>C (p.Leu1940Pro) | ex41 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5821C>T (p.His1941Tyr) | ex41 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5836−2A>G (−) | int41 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5836−2del (−) | int41 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5843C>T (p.Pro1948Leu) | ex42 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5844A>G (p.(=)) | ex42 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5881G>A | ex42 | Photoreceptors | Int 26 (NG_009073.1) | ~30-250 nts | Ex 27-50 [c.3863G | 3' |
| | | (p.Gly1961Arg) | | | | complementary | p.Gly1289-2] through | |
| | | | | | | to target intron | [c.6822A p.Stop2274] | |
| | | | | | | | (NM_000350.2) | |
| ABCA4 | STGD, | c.5882G>A (p.Gly1961Glu) | ex42 | Photoreceptors | Int 26 (NG_009073.1) | ~30-250 nts | Ex 27-50 [c.3863G | 3' |
| | AMD, | | | | | complementary | p.Gly1289-2] through | |
| | ARRP, | | | | | to target intron | [c.6822A p.Stop2274] | |
| | CRD | | | | | | (NM_000350.2) | |
| ABCA4 | STGD | c.5885T>A (p.Val1962Asp) | ex42 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | ARRP | c.5888delG (p.Arg1963fs) | ex42 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5898+1G>T (−) | int42 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5898+1G>A (−) | int42 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5898+3delG (−) | int42 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5905delG (p.Gly1969fs) | ex43 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | AMD | c.5908C>T (p.Leu1970Phe) | ex43 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5912T>G (p.Leu1971Arg) | ex43 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5914G>A | ex43 | Photoreceptors | Int 26 (NG_009073.1) | ~30-250 nts | Ex 27-50 [c.3863G | 3' |
| | | (p.Gly1972Arg) | | | | complementary | p.Gly1289-2] through | |
| | | | | | | to target intron | [c.6822A p.Stop2274] | |
| | | | | | | | (NM_000350.2) | |
| ABCA4 | ARRP, | c.5917delG (p.Val1973*) | ex43 | Photoreceptors | Int 26 (NG_009073.1) | ~30-250 nts | Ex 27-50 [c.3863G | 3' |
| | CRD | | | | | complementary | p.Gly1289-2] through | |
| | | | | | | to target intron | [c.6822A p.Stop2274] | |
| | | | | | | | (NM_000350.2) | |
| ABCA4 | STGD | c.5923G>C (p.Gly1975Arg) | ex43 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5929G>A (p.Gly1977Ser) | ex43 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5932delA (p.Thr1979fs) | ex43 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5936C>T (p.Thr1979Ile) | ex43 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.5961_5964del4 | ex43 | Photoreceptors | Int 26 (NG_009073.1) | ~30-250 nts | Ex 27-50 [c.3863G | 3' |
| | | (p.Asp1988fs) | | | | complementary | p.Gly1289-2] through | |
| | | | | | | to target intron | [c.6822A p.Stop2274] | |
| | | | | | | | (NM_000350.2) | |
| ABCA4 | STGD | c.6005+1G>T (−) | int43 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.6079C>T (p.Leu2027Phe) | ex44 | Photoreceptors | Int 26 (NG_009073.1) | ~30-250 nts | Ex 27-50 [c.3863G | 3' |
| | AMD, | | | | | complementary | p.Gly1289-2] through | |
| | CRD | | | | | to target intron | [c.6822A p.Stop2274] | |
| | | | | | | | (NM_000350.2) | |
| ABCA4 | CRD | c.6088C>T (p.Arg2030*) | ex44 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6089G>A | ex44 | Photoreceptors | Int 26 (NG_009073.1) | ~30-250 nts | Ex 27-50 [c.3863G | 3' |
| | | (p.Arg2030Gln) | | | | complementary | p.Gly1289-2] through | |
| | | | | | | to target intron | [c.6822A p.Stop2274] | |
| | | | | | | | (NM_000350.2) | |
| ABCA4 | STGD | c.6104T>C (p.Leu2035Pro) | ex44 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6112C>T (p.Arg2038Trp) | ex44 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6118C>T (p.Arg2040*) | ex44 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6122G>A | ex44 | Photoreceptors | Int 26 (NG_009073.1) | ~30-250 nts | Ex 27-50 [c.3863G | 3' |
| | | (p.Gly2041Asp) | | | | complementary | p.Gly1289-2] through | |
| | | | | | | to target intron | [c.6822A p.Stop2274] | |
| | | | | | | | (NM_000350.2) | |
| ABCA4 | STGD | c.6140T>A (p.Ile2047Asn) | ex44 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6147+2T>A (−) | int44 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.6148G>C (p.Val2050Leu) | ex45 | Photoreceptors | Int 26 (NG_009073.1) | ~30-250 nts | Ex 27-50 [c.3863G | 3' |
| | ARRP, | | | | | complementary | p.Gly1289-2] through | |
| | AMD | | | | | to target intron | [c.6822A p.Stop2274] | |
| | | | | | | | (NM_000350.2) | |
| ABCA4 | STGD | c.6166A>T (p.Lys2056*) | ex45 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6179T>G (p.Leu2060Arg) | ex45 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6190G>C (p.Ala2064Pro) | ex45 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6212A>T (p.Tyr2071Phe) | ex45 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6220G>A (p.Gly2074Ser) | ex45 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6229C>T (p.Arg2077Trp) | ex45 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6229C>G (p.Arg2077Gly) | ex45 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6238 6239del | ex45 | Photoreceptors | Int 26 (NG_009073.1) | ~30-250 nts | Ex 27-50 [c.3863G | 3' |
| | | (p.Ser2080fs) | | | | complementary | p.Gly1289-2] through | |
| | | | | | | to target intron | [c.6822A p.Stop2274] | |
| | | | | | | | (NM_000350.2) | |
| ABCA4 | STGD, | c.6286G>A (p.Glu2096Lys) | ex46 | Photoreceptors | Int 26 (NG_009073.1) | ~30-250 nts | Ex 27-50 [c.3863G | 3' |
| | AMD | | | | | complementary | p.Gly1289-2] through | |
| | | | | | | to target intron | [c.6822A p.Stop2274] | |
| | | | | | | | (NM_000350.2) | |
| ABCA4 | STGD | c.6300delG (p.Met2101fs) | ex46 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6306C>A | ex46 | Photoreceptors | Int 26 (NG_009073.1) | ~30-250 nts | Ex 27-50 [c.3863G | 3' |
| | | (p.Asp2102Glu) | | | | complementary | p.Gly1289-2] through | |
| | | | | | | to target intron | [c.6822A p.Stop2274] | |
| | | | | | | | (NM_000350.2) | |
| ABCA4 | STGD | c.6316C>T (p.Arg2106Cys) | ex46 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6317_6323del7 | ex46 | Photoreceptors | Int 26 (NG_009073.1) | ~30-250 nts | Ex 27-50 [c.3863G | 3' |
| | | (p.Arg2107fs) | | | | complementary | p.Gly1289-2] through | |
| | | | | | | to target intron | [c.6822A p.Stop2274] | |
| | | | | | | | (NM_000350.2) | |
| ABCA4 | STGD | c.6319C>T (p.Arg2107Cys) | ex46 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.6320G>A (p.Arg2107His) | ex46 | Photoreceptors | Int 26 (NG_009073.1) | ~30-250 nts | Ex 27-50 [c.3863G | 3' |
| | AMD | | | | | complementary | p.Gly1289-2] through | |
| | | | | | | to target intron | [c.6822A p.Stop2274] | |
| | | | | | | | (NM_000350.2) | |
| ABCA4 | STGD | c.6320G>C (p.Arg2107Pro) | ex46 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6329G>A (p.Trp2110*) | ex46 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6339C>G (p.Ile2113Met) | ex46 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6352delA (p.Arg2118fs) | ex46 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6383A>G (p.His2128Arg) | ex46 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6386+2C>G (−) | int46 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6387−1G>T (−) | int46 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6391G>A (p.Glu2131Lys) | ex47 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6410G>A (p.Cys2137Tyr) | ex47 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6411T>A (p.Cys2137*) | ex47 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6415C>T (p.Arg2139Trp) | ex47 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6419T>A (p.Leu2140Gln) | ex47 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6437G>A | ex47 | Photoreceptors | Int 26 (NG_009073.1) | ~30-250 nts | Ex 27-50 [c.3863G | 3' |
| | | (p.Gly2146Asp) | | | | complementary | p.Gly1289-2] through | |
| | | | | | | to target intron | [c.6822A p.Stop2274] | |
| | | | | | | | (NM_000350.2) | |
| ABCA4 | STGD | c.6445C>T (p.Arg2149*) | ex47 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6446G>T (p.Arg2149Leu) | ex47 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6448T>C (p.Cys2150Arg) | ex47 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | CRD | c.6449G>A (p.Cys2150Tyr) | ex47 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6479A>G | ex47 | Photoreceptors | Int 26 (NG_009073.1) | ~30-250 nts | Ex 27-50 [c.3863G | 3' |
| | | (p.Lys2160Arg) | | | | complementary | p.Gly1289-2] through | |
| | | | | | | to target intron | [c.6822A p.Stop2274] | |
| | | | | | | | (NM_000350.2) | |
| ABCA4 | STGD | c.6479+1G>A (−) | int47 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6479+1G>C (−) | int47 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | AMD | c.6498C>G (p.Ile2166Met) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6498C>T (p.Ile2166p.(=)) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6515A>G | ex48 | Photoreceptors | Int 26 (NG_009073.1) | ~30-250 nts | Ex 27-50 [c.3863G | 3' |
| | | (p.Lys2172Arg) | | | | complementary | p.Gly1289-2] through | |
| | | | | | | to target intron | [c.6822A p.Stop2274] | |
| | | | | | | | (NM_000350.2) | |
| ABCA4 | STGD, | c.6519 6529del11 | ex48 | Photoreceptors | Int 26 (NG_009073.1) | ~30-250 nts | Ex 27-50 [c.3863G | 3' |
| | AMD | (p.Lys2175fs) | | | | complementary | p.Gly1289-2] through | |
| | | | | | | to target intron | [c.6822A p.Stop2274] | |
| | | | | | | | (NM_000350.2) | |
| ABCA4 | ARRP | c.6529G>A | ex48 | Photoreceptors | Int 26 (NG_009073.1) | ~30-250 nts | Ex 27-50 [c.3863G | 3' |
| | | (p.Asp2177Asn) | | | | complementary | p.Gly1289-2] through | |
| | | | | | | to target intron | [c.6822A p.Stop2274] | |
| | | | | | | | (NM_000350.2) | |
| ABCA4 | STGD | c.6543_6578del36 | ex48 | Photoreceptors | Int 26 (NG_009073.1) | ~30-250 nts | Ex 27-50 [c.3863G | 3' |
| | | (p.Leu2182_Phe2193del) | | | | complementary | p.Gly1289-2] through | |
| | | | | | | to target intron | [c.6822A p.Stop2274] | |
| | | | | | | | (NM_000350.2) | |
| ABCA4 | STGD | 6548_6549insTGAA | ex48 | Photoreceptors | Int 26 (NG_009073.1) | ~30-250 nts | Ex 27-50 [c.3863G | 3' |
| | | (p.Pro2184fs) | | | | complementary | p.Gly1289-2] through | |
| | | | | | | to target intron | [c.6822A p.Stop2274] | |
| | | | | | | | (NM_000350.2) | |
| ABCA4 | \|STGD | c.6559C>T (p.Gln2187*) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6560A>C (p.Gln2187Pro) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6563T>C (p.Phe2188Ser) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | AMD | c.6568delC (p.Gln2190fs) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | CRD | c.6601_6602delAG | ex48 | Photoreceptors | Int 26 (NG_009073.1) | ~30-250 nts | Ex 27-50 [c.3863G | 3' |
| | | (p.Arg2201fs) | | | | complementary | p.Gly1289-2] through | |
| | | | | | | to target intron | [c.6822A p.Stop2274] | |
| | | | | | | | (NM_000350.2) | |
| ABCA4 | STGD | c.6609C>A (p.Tyr2203*) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6658C>T (p.Gln2220*) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6662T>C (p.Leu2221Pro) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6686T>C (p.Leu2229Pro) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6707_6714del | ex48 | Photoreceptors | Int 26 (NG_009073.1) | ~30-250 nts | Ex 27-50 [c.3863G | 3' |
| | | (p.Val2236fs) | | | | complementary | p.Gly1289-2] through | |
| | | | | | | to target intron | [c.6822A p.Stop2274] | |
| | | | | | | | (NM_000350.2) | |
| ABCA4 | STGD | c.6709_6710insG | ex48 | Photoreceptors | Int 26 (NG_009073.1) | ~30-250 nts | Ex 27-50 [c.3863G | 3' |
| | | (p.Thr2237fs) | | | | complementary | p.Gly1289-2] through | |
| | | | | | | to target intron | [c.6822A p.Stop2274] | |
| | | | | | | | (NM_000350.2) | |
| ABCA4 | STGD | c.6710_6711insA | ex48 | Photoreceptors | Int 26 (NG_009073.1) | ~30-250 nts | Ex 27-50 [c.3863G | 3' |
| | | (p.Gln2238fs) | | | | complementary | p.Gly1289-2] through | |
| | | | | | | to target intron | [c.6822A p.Stop2274] | |
| | | | | | | | (NM_000350.2) | |
| ABCA4 | STGD | c.6712C>T (p.Gln2238*) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6718A>G (p.Thr2240Ala) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6721C>G (p.Leu2241Val) | ex48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6729+1G>A (−) | int48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6729+5_6729+19del15 (−) | int48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6730-10_6730-2de19 (−) | int48 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6730-16_6757del (−) | int48/ | Photoreceptors | Int 26 (NG_009073.1) | ~30-250 nts | Ex 27-50 [c.3863G | 3' |
| | | | ex49 | | | complementary | p.Gly1289-2] through | |
| | | | | | | to target intron | [c.6822A p.Stop2274] | |
| | | | | | | | (NM_000350.2) | |
| ABCA4 | STGD | c.6748delA (p.Lys2250fs) | ex49 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6757A>G (p.Thr2253Ala) | ex49 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD, | c.6764G>T (p.Ser2255Ile) | ex49 | Photoreceptors | Int 26 (NG_009073.1) | ~30-250 nts | Ex 27-50 [c.3863G | 3' |
| | AMD | | | | | complementary | p.Gly1289-2] through | |
| | | | | | | to target intron | [c.6822A p.Stop2274] | |
| | | | | | | | (NM_000350.2) | |
| ABCA4 | STGD | c.6788G>T (p.Arg2263Leu) | ex49 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |
| ABCA4 | STGD | c.6817-1G>A ( ) | int49 | Photoreceptors | Int 26 (NG_009073.1) | ~30-250 nts | Ex 27-50 [c.3863G | 3' |
| | | | | | | complementary | p.Gly1289-2] through | |
| | | | | | | to target intron | [c.6822A p.Stop2274] | |
| | | | | | | | (NM_000350.2) | |

TABLE 2

| | | | | | RTM | | |
|---|---|---|---|---|---|---|---|
| Target | | | | | Binding | | Splice |
| Ocular | | | | | Domain | | Site |
| Gene | Ocular Disease | Mutation | Target Cells | Intron/SEQ | Seq | Exon/Seq | Seq |
| CEP290 | Meckel Syndrome | c.3043G > T | Multi-organ | Int 26 | ~30-250 nts | Ex 27-54 [c.2992T p.Cys998] | 3′ |
| | | | | (NG_008417.1) | complementary | through | |
| | | | | | to target intron | [c.7440A p.Stop2480] | |
| | | | | | | (NM_025114.3) | |
| CEP290 | Meckel Syndrome | c.3104 − 1G > A | multi-organ | Int 26 | ~30-250 nts | Ex 27-54 [c.2992T p.Cys998] | 3′ |
| | | | | (NG_008417.1) | complementary | through | |
| | | | | | to target intron | [c.7440A p.Stop2480] | |
| | | | | | | (NM_025114.3) | |
| CEP290 | Joubert Syndrome: LCA & | c.3104 − 2A > G | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Meckel Syndrome | c.3175del | Photoreceptors, | Int 26 | ~30-250 nts | Ex 27-54 [c.2992T p.Cys998] | 3′ |
| | | | Kidney tubular | (NG_008417.1) | complementary | through | |
| | | | epitheliuam; CNS | | to target intron | [c.7440A p.Stop2480] | |
| | | | | | | (NM_025114.3) | |
| CEP290 | Joubert Syndrome: LCA & | c.3175dup | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.3176del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.3178delA | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.3292G > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.3310 − 1G > C | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.3310 − a_ | Photoreceptors, | Int 26 | ~30-250 nts | Ex 27-54 [c.2992T p.Cys998] | 3′ |
| | Nephronophthisis & CNS | 3310delinsAA | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.3422dup | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Meckel Syndrome | c.3793C > T | Multi-organ | Int 26 | ~30-250 nts | Ex 27-54 [c.2992T p.Cys998] | 3′ |
| | | | | (NG_008417.1) | complementary | through | |
| | | | | | to target intron | [c.7440A p.Stop2480] | |
| | | | | | | (NM_025114.3) | |
| CEP290 | Joubert Syndrome: LCA & | c.3802C > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.3811C > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.3814C > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.3922C > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.4001del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.4028del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.4114_4115del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Meckel Syndrome | c.4115_4116del | multi-organ | Int 26 | ~30-250 nts | Ex 27-54 [c.2992T p.Cys998] | 3′ |
| | | | | (NG_008417.1) | complementary | through | |
| | | | | | to target intron | [c.7440A p.Stop2480] | |
| | | | | | | (NM_025114.3) | |
| CEP290 | Joubert Syndrome: LCA & | c.4195 − 1G > A | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Senior Loken Syndrome: | c.4452_ | Photoreceptors, | Int 26 | ~30-250 nts | Ex 27-54 [c.2992T p.Cys998] | 3′ |
| | LCA & Nephronophthisis | 4455delAGAA | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.4656del | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | LCA | c.4661_4663del | Photoreceptors | Int 26 | ~30-250 nts | Ex 27-54 [c.2992T p.Cys998] | 3′ |
| | | | | (NG_008417.1) | complementary | through | |
| | | | | | to target intron | [c.7440A p.Stop2480] | |
| | | | | | | (NM_025114.3) | |
| CEP290 | Joubert Syndrome: LCA & | c.4723A > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.4732G > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.4771C > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.4791_4794del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.4882C > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.4962_4963del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.4965_4966del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.4966G > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.5046del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.5163del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.5182G > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.5218C > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Senior Loken Syndrome: | c.5226 + | Photoreceptors, | Int 26 | ~30-250 nts | Ex 27-54 [c.2992T p.Cys998] | 3′ |
| | LCA & Nephronophthisis | 5_8delGTAA | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.5256_5257del | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.5434_5435del | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.5445 − | Photoreceptors, | Int 26 | ~30-250 nts | Ex 27-54 [c.2992T p.Cys998] | 3′ |
| | LCA & Nephronophthisis | 8delAACT | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.5493del | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.5515_5518del | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.5519_5537del | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.5587 − 1G > C | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.5611_5614del | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Meckel Syndrome | c.5649_ | multi-organ | Int 26 | ~30-250 nts | Ex 27-54 [c.2992T p.Cys998] | 3′ |
| | | 5650insA | | (NG_008417.1) | complementary | through | |
| | | | | | to target intron | [c.7440A p.Stop2480] | |
| | | | | | | (NM_025114.3) | |
| CEP290 | Senior Loken Syndrome: | c.5649dup | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Joubert Syndrome: LCA & | c.5722G > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.5734del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.5776C > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.5813_5817del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.5824C > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Meckel Syndrome | c.5850del | multi-organ | Int 26 | ~30-250 nts | Ex 27-54 [c.2992T p.Cys998] | 3′ |
| | | | | (NG_008417.1) | complementary | through | |
| | | | | | to target intron | [c.7440A p.Stop2480] | |
| | | | | | | (NM_025114.3) | |
| CEP290 | Joubert Syndrome: LCA & | c.5865_ | Photoreceptors, | Int 26 | ~30-250 nts | Ex 27-54 [c.2992T p.Cys998] | 3′ |
| | Nephronophthisis & CNS | 5867delinsGG | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.5866G > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Senior Loken Syndrome: | c.5932C > T | photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epithelium | | to target intron | [c.7440A p.Stop2480] | |
| | | | | | | (NM_025114.3) | |
| CEP290 | Joubert Syndrome: LCA & | c.6031C > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.6072C > A | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Senior Loken Syndrome: | c.6271 − 8T > G | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.6277del | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.6604del | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Joubert Syndrome: LCA & | c.6869del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | c.6870del | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Senior Loken Syndrome: | c.7318_ | Photoreceptors, | Int 26 | ~30-250 nts | Ex 27-54 [c.2992T p.Cys998] | 3′ |
| | LCA & Nephronophthisis | 7321dup | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.7341dup | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c.7366_7369del | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | Senior Loken Syndrome: | c5226 + 1G > A | Photoreceptors, | Int 26 | ~30-250 nts | Ex 27-54 [c.2992T p.Cys998] | 3′ |
| | LCA & Nephronophthisis | | Kidney tubular | (NG_008417.1) | complementary | through | |
| | | | epitheliuam | | to target intron | [c.7440A p.Stop2480] | |
| | | | | | | (NM_025114.3) | |
| CEP290 | LCA | c5777G > C | Photoreceptors | Int 26 | ~30-250 nts | Ex 27-54 [c.2992T p.Cys998] | 3′ |
| | | | | (NG_008417.1) | complementary | through | |
| | | | | | to target intron | [c.7440A p.Stop2480] | |
| | | | | | | (NM_025114.3) | |
| CEP290 | Joubert Syndrome: LCA & | c5941G > T | Photoreceptors, | Int 26 | ~30-250 nts | Ex 27-54 [c.2992T p.Cys998] | 3′ |
| | Nephronophthisis & CNS | | Kidney tubular | (NG_008417.1) | complementary | through | |
| | anomalies | | epitheliuam; CNS | | to target intron | [c.7440A p.Stop2480] | |
| | | | | | | (NM_025114.3) | |
| CEP290 | Joubert Syndrome: LCA & | 1984C | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.103 − 13_ | Photoreceptors, | Int 26 | ~30-250 nts | Ex 1-26 [c.1A p.Met1] | 5′ |
| | Nephronophthisis & CNS | 103 − 18del | Kidney tubular | (NG_008417.1) | complementary | through | |
| | anomalies | | epitheliuam; CNS | | to target intron | [c.2991G p.Glu997] | |
| CEP290 | Joubert Syndrome: LCA & | c.1066 − 1G > A | Photoreceptors, | Int 26 | ~30-250 nts | (NM_025114.3) | 5′ |
| | Nephronophthisis & CNS | | Kidney tubular | (NG_008417.1) | complementary | Ex 1-26 [c.1A p.Met1] | |
| | anomalies | | epitheliuam; CNS | | to target intron | through | |
| CEP290 | Joubert Syndrome: LCA & | c.1189 + 1G > A | Photoreceptors, | Int 26 | ~30-250 nts | [c.2991G p.Glu997] | 5′ |
| | Nephronophthisis & CNS | | Kidney tubular | (NG_008417.1) | complementary | (NM_025114.3) | |
| | anomalies | | epitheliuam; CNS | | to target intron | Ex 1-26 [c.1A p.Met1] | |
| CEP290 | Meckel Syndrome | c.1219_1220del | multi-organ | Int 26 | ~30-250 nts | through | 5′ |
| | | | | (NG_008417.1) | complementary | [c.2991G p.Glu997] | |
| | | | | | to target intron | (NM_025114.3) | |
| CEP290 | Joubert Syndrome: LCA & | c.1260_1264del | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.1361del | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Senior Loken Syndrome: | c.136G > T | Photoreceptors, | Int 26 | ~30-250 nts | Ex 1-26 [c.1A p.Met1] | 5′ |
| | LCA & Nephronophthisis | | Kidney tubular | (NG_008417.1) | complementary | through | |
| | | | epitheliuam | | to target intron | [c.2991G p.Glu997] | |
| | | | | | | (NM_025114.3) | |
| CEP290 | Senior Loken Syndrome: | c.1419_1423del | photoreceptors, | Int 26 | ~30-250 nts | Ex 1-26 [c.1A p.Met1] | 5′ |
| | LCA & Nephronophthisis | | Kidney tubular | (NG_008417.1) | complementary | through | |
| | | | epitheliuam | | to target intron | [c.2991G p.Glu997] | |
| | | | | | | (NM_025114.3) | |
| CEP290 | Joubert Syndrome: LCA & | c.1429T | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.164_167del | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.1645C > T | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.1666del | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.1682_1683del | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.1709C > G | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.1711 + 5A > G | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.180 + 1G > T | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Meckel Syndrome | c.180 + 2T > A | multi-organ | Int 26 | ~30-250 nts | Ex 1-26 [c.1A p.Met1] | 5′ |
| | | | | (NG_008417.1) | complementary | through | |
| | | | | | to target intron | [c.2991G p.Glu997] | |
| | | | | | | (NM_025114.3) | |
| CEP290 | Joubert Syndrome: LCA & | c.1824G > A | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.1830delA | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.1859_1862del | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Meckel Syndrome | c.1860_1861del | multi-organ | Int 26 | ~30-250 nts | Ex 1-26 [c.1A p.Met1] | 5′ |
| | | | | (NG_008417.1) | complementary | through | |
| | | | | | to target intron | [c.2991G p.Glu997] | |
| | | | | | | (NM_025114.3) | |
| CEP290 | Joubert Syndrome: LCA & | c.1910 − 2A > C | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Joubert Syndrome: LCA & | c.1936C > T | Photoreceptors, | Int 26 | ~30-250 nts | Ex 1-26 [c.1A p.Met1] | 5′ |
| | Nephronophthisis & CNS | | Kidney tubular | (NG_008417.1) | complementary | through | |
| | anomalies | | epitheliuam; CNS | | to target intron | [c.2991G p.Glu997] | |
| | | | | | | (NM_025114.3) | |
| CEP290 | Meckel Syndromec kidneys, | c.1984C > T | Multi-organ | Int 26 | ~30-250 nts | Ex 1-26 [c.1A p.Met1] | 5′ |
| | polydactyly, hepatic fibrosis, | | | (NG_008417.1) | complementary | through | |
| | LCA) (encephalocele, | | | | to target intron | [c.2991G p.Glu997] | |
| | polycyst | | | | ~30-250 nts | (NM_025114.3) | |
| | | | | | | Ex 1-26 [c.1A p.Met1] | |
| CEP290 | LCA | c.1985A > T | Photoreceptors | Int 26 | complementary | through | 5′ |
| | | | | (NG_008417.1) | to target intron | [c.2991G p.Glu997] | |
| | | | | | ~30-250 nts | (NM_025114.3) | |
| | | | | | | Ex 1-26 [c.1A p.Met1] | |
| CEP290 | Joubert Syndrome: LCA & | c.1987A > T | Photoreceptors, | Int 26 | complementary | through | 5′ |
| | Nephronophthisis & CNS | | Kidney tubular | (NG_008417.1) | to target intron | [c.2991G p.Glu997] | |
| | anomalies | | epitheliuam; CNS | | ~30-250 nts | (NM_025114.3) | |
| | | | | | | Ex 1-26 [c.1A p.Met1] | |
| CEP290 | LCA | c.1991A > G | Photoreceptors | Int 26 | complementary | through | 5′ |
| | | | | (NG_008417.1) | to target intron | [c.2991G p.Glu997] | |
| | | | | | ~30-250 nts | (NM_025114.3) | |
| | | | | | | Ex 1-26 [c.1A p.Met1] | |
| CEP290 | Joubert Syndrome: LCA & | c.1992del | Photoreceptors, | Int 26 | complementary | Ex 1-26 [c.1A p.Met1] | 5′ |
| | Nephronophthisis & CNS | | Kidney tubular | (NG_008417.1) | to target intron | through | |
| | anomalies | | epitheliuam; CNS | | ~30-250 nts | [c.2991G p.Glu997] | |
| | | | | | | (NM_025114.3) | |
| CEP290 | LCA | c.1A > G | Photoreceptors | Int 26 | complementary | Ex 1-26 [c.1A p.Met1] | 5′ |
| | | | | (NG_008417.1) | to target intron | through | |
| | | | | | ~30-250 nts | [c.2991G p.Glu997] | |
| | | | | | | (NM_025114.3) | |
| CEP290 | Senior Loken Syndrome: | c.2118_2122dup | Photoreceptors, | Int 26 | complementary | Ex 1-26 [c.1A p.Met1] | 5′ |
| | LCA & Nephronophthisis | | Kidney tubular | (NG_008417.1) | to target intron | through | |
| | | | epithelium | | ~30-250 nts | [c.2991G p.Glu997] | |
| | | | | | | (NM_025114.3) | |
| CEP290 | LCA | c.21G > T | Photoreceptors | Int 26 | complementary | Ex 1-26 [c.1A p.Met1] | 5′ |
| | | | | (NG_008417.1) | to target intron | through | |
| | | | | | ~30-250 nts | [c.2991G p.Glu997] | |
| | | | | | | (NM_025114.3) | |
| CEP290 | Joubert Syndrome: LCA & | c.2213delT | Photoreceptors, | Int 26 | complementary | Ex 1-26 [c.1A p.Met1] | 5′ |
| | Nephronophthisis & CNS | | Kidney tubular | (NG_008417.1) | to target intron | through | |
| | anomalies | | epitheliuam; CNS | | ~30-250 nts | [c.2991G p.Glu997] | |
| | | | | | | (NM_025114.3) | |
| CEP290 | Senior Loken Syndrome: | c.2218 − | Photoreceptors, | Int 26 | complementary | Ex 1-26 [c.1A p.Met1] | 5′ |
| | LCA & Nephronophthisis | 15_2220del | Kidney tubular | (NG_008417.1) | to target intron | through | |
| | | | epithelium | | ~30-250 nts | [c.2991G p.Glu997] | |
| | | | | | | (NM_025114.3) | |
| CEP290 | Senior Loken Syndrome: | c.2218 − 2A > C | Photoreceptors, | Int 26 | complementary | Ex 1-26 [c.1A p.Met1] | 5′ |
| | LCA & Nephronophthisis | | Kidney tubular | (NG_008417.1) | to target intron | through | |
| | | | epithelium | | ~30-250 nts | [c.2991G p.Glu997] | |
| | | | | | | (NM_025114.3) | |
| CEP290 | Senior Loken Syndrome: | c.2218 − | Photoreceptors, | Int 26 | complementary | Ex 1-26 [c.1A p.Met1] | 5′ |
| | LCA & Nephronophthisis | 4_2222del | Kidney tubular | (NG_008417.1) | to target intron | through | |
| | | | epithelium | | ~30-250 nts | [c.2991G p.Glu997] | |
| | | | | | | (NM_025114.3) | |
| CEP290 | Joubert Syndrome: LCA & | c.2505_ | Photoreceptors, | Int 26 | complementary | Ex 1-26 [c.1A p.Met1] | 5′ |
| | Nephronophthisis & CNS | 2506delAG | Kidney tubular | (NG_008417.1) | to target intron | through | |
| | anomalies | | epitheliuam; CNS | | ~30-250 nts | [c.2991G p.Glu997] | |
| | | | | | | (NM_025114.3) | |
| CEP290 | Senior Loken Syndrome: | c.265dup | Photoreceptors, | Int 26 | complementary | Ex 1-26 [c.1A p.Met1] | 5′ |
| | LCA & Nephronophthisis | | Kidney tubular | (NG_008417.1) | to target intron | through | |
| | | | epithelium | | ~30-250 nts | [c.2991G p.Glu997] | |
| | | | | | | (NM_025114.3) | |
| CEP290 | Senior Loken Syndrome: | c.270_ | Photoreceptors, | Int 26 | complementary | Ex 1-26 [c.1A p.Met1] | 5′ |
| | LCA & Nephronophthisis | 274delAGTAA | Kidney tubular | (NG_008417.1) | to target intron | through | |
| | | | epithelium | | ~30-250 nts | [c.2991G p.Glu997] | |
| | | | | | | (NM_025114.3) | |
| CEP290 | Senior Loken Syndrome: | c.287del | Photoreceptors, | Int 26 | complementary | Ex 1-26 [c.1A p.Met1] | 5′ |
| | LCA & Nephronophthisis | | Kidney tubular | (NG_008417.1) | to target intron | through | |
| | | | epithelium | | ~30-250 nts | [c.2991G p.Glu997] | |
| | | | | | | (NM_025114.3) | |
| CEP290 | Meckel Syndrome | c.2906dupA | multi-organ | Int 26 | complementary | Ex 1-26 [c.1A p.Met1] | 5′ |
| | | | | (NG_008417.1) | to target intron | through | |
| | | | | | ~30-250 nts | [c.2991G p.Glu997] | |
| | | | | | | (NM_025114.3) | |
| CEP290 | LCA | c.2915T > G | Photoreceptors | Int 26 | complementary | Ex 1-26 [c.1A p.Met1] | 5′ |
| | | | | (NG_008417.1) | to target intron | through | |
| | | | | | ~30-250 nts | [c.2991G p.Glu997] | |
| | | | | | | (NM_025114.3) | |
| CEP290 | LCA | c.2991 + | Photoreceptors | Int 26 | complementary | Ex 1-26 [c.1A p.Met1] | 5′ |
| | | 1655A > G | | (NG_008417.1) | to target intron | through | |
| | | | | | ~30-250 nts | [c.2991G p.Glu997] | |
| | | | | | | (NM_025114.3) | |
| CEP290 | LCA | c.2T > G | Photoreceptors | Int 26 | complementary | Ex 1-26 [c.1A p.Met1] | 5′ |
| | | | | (NG_008417.1) | to target intron | through | |
| | | | | | ~30-250 nts | [c.2991G p.Glu997] | |
| | | | | | | (NM_025114.3) | |
| CEP290 | Joubert Syndrome: LCA & | c.322C > T | Photoreceptors, | Int 26 | complementary | Ex 1-26 [c.1A p.Met1] | 5′ |
| | Nephronophthisis & CNS | | Kidney tubular | (NG_008417.1) | to target intron | through | |
| | anomalies | | epitheliuam; CNS | | ~30-250 nts | [c.2991G p.Glu997] | |
| | | | | | | (NM_025114.3) | |
| CEP290 | Meckel Syndrome | c.381_ | Multi-organ | Int 26 | complementary | Ex 1-26 [c.1A p.Met1] | 5′ |
| | | 382delinsT | | (NG_008417.1) | to target intron | through | |
| | | | | | ~30-250 nts | [c.2991G p.Glu997] | |
| | | | | | | (NM_025114.3) | |
| CEP290 | Senior Loken Syndrome: | c.384_385del | Photoreceptors, | Int 26 | complementary | Ex 1-26 [c.1A p.Met1] | 5′ |
| | LCA & Nephronophthisis | | Kidney tubular | (NG_008417.1) | to target intron | through | |
| | | | epithelium | | ~30-250 nts | [c.2991G p.Glu997] | |
| | | | | | | (NM_025114.3) | |
| CEP290 | Meckel Syndrome | c.384_387del | Multi-organ | Int 26 | complementary | Ex 1-26 [c.1A p.Met1] | 5′ |
| | | | | (NG_008417.1) | to target intron | through | |
| | | | | | ~30-250 nts | [c.2991G p.Glu997] | |
| | | | | | | (NM_025114.3) | |
| CEP290 | Meckel Syndrome | c.387delTAGA | multi-organ | Int 26 | complementary | Ex 1-26 [c.1A p.Met1] | 5′ |
| | | | | (NG_008417.1) | to target intron | through | |
| | | | | | ~30-250 nts | [c.2991G p.Glu997] | |
| | | | | | | (NM_025114.3) | |
| CEP290 | Senior Loken Syndrome: | c.437del | Photoreceptors, | Int 26 | complementary | Ex 1-26 [c.1A p.Met1] | 5′ |
| | LCA & Nephronophthisis | | Kidney tubular | (NG_008417.1) | to target intron | through | |
| | | | epithelium | | ~30-250 nts | [c.2991G p.Glu997] | |
| | | | | | | (NM_025114.3) | |
| CEP290 | Joubert Syndrome: LCA & | c.451C > T | Photoreceptors, | Int 26 | complementary | Ex 1-26 [c.1A p.Met1] | 5′ |
| | Nephronophthisis & CNS | | Kidney tubular | (NG_008417.1) | to target intron | through | |
| | anomalies | | epitheliuam; CNS | | ~30-250 nts | [c.2991G p.Glu997] | |
| | | | | | | (NM_025114.3) | |
| CEP290 | Joubert Syndrome: LCA & | c.566C > G | Photoreceptors, | Int 26 | complementary | Ex 1-26 [c.1A p.Met1] | 5′ |
| | Nephronophthisis & CNS | | Kidney tubular | (NG_008417.1) | to target intron | through | |
| | anomalies | | epitheliuam; CNS | | ~30-250 nts | [c.2991G p.Glu997] | |
| | | | | | | (NM_025114.3) | |
| CEP290 | Meckel Syndrome | c.613C > T | Multi-organ | Int 26 | complementary | Ex 1-26 [c.1A p.Met1] | 5′ |
| | | | | (NG_008417.1) | to target intron | through | |
| | | | | | ~30-250 nts | [c.2991G p.Glu997] | |
| | | | | | | (NM_025114.3) | |
| CEP290 | Senior Loken Syndrome: | c.679_680del | Photoreceptors, | Int 26 | complementary | Ex 1-26 [c.1A p.Met1] | 5′ |
| | LCA & Nephronophthisis | | Kidney tubular | (NG_008417.1) | to target intron | through | |
| | | | epithelium | | ~30-250 nts | [c.2991G p.Glu997] | |
| | | | | | | (NM_025114.3) | |
| CEP290 | LCA | c.829G > C | Photoreceptors | Int 26 | complementary | Ex 1-26 [c.1A p.Met1] | 5′ |
| | | | | (NG_008417.1) | to target intron | through | |
| | | | | | ~30-250 nts | [c.2991G p.Glu997] | |
| | | | | | | (NM_025114.3) | |
| CEP290 | Joubert Syndrome: LCA & | IVS10 − | Photoreceptors, | Int 26 | complementary | Ex 1-26 [c.1A p.Met1] | 5′ |
| | Nephronophthisis & CNS | 11_12insG | Kidney tubular | (NG_008417.1) | to target intron | through | |
| | anomalies | | epitheliuam; CNS | | ~30-250 nts | [c.2991G p.Glu997] | |
| | | | | | | (NM_025114.3) | |
| CEP290 | Joubert Syndrome: LCA & | IVS26 + | Photoreceptors, | Int 26 | complementary | Ex 1-26 [c.1A p.Met1] | 5′ |
| | Nephronophthisis & CNS | 1655A > G | Kidney tubular | (NG_008417.1) | to target intron | through | |
| | anomalies | | epitheliuam; CNS | | ~30-250 nts | [c.2991G p.Glu997] | |
| | | | | | | (NM_025114.3) | |

TABLE 3

| | | | | | | RTM | | |
|---|---|---|---|---|---|---|---|---|
| Target | | | | | | | | |
| Ocular | Ocular | | | | | Binding Domain | | Site |
| Gene | Disease | Mutation | Protein change | Target Cells | Intron/SEQ | Seq | Exon/Seq | Seq |
| MYO7A | Usher | c.4450C > T | p.L1484F | RPE, Photoreceptors, Cochlear hair | Int 32 (NG 009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.4475C > T | p.A1492V | RPE, Photoreceptors, Cochlear hair | Int 32 (NG_009086.1) | ~~ 30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.4697C > T | p.T1566M | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.4740C > A | p.Y1580X | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.4805G > A | p.R1602Q | RPE, Photoreceptors, Cochlear hair | Int 32 (NG 009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.4882G > T | p.A1628S | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.4996A > T | p.S1666C | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5101C > T | p.R1701X | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5146G > T | p.E1716X | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5156A > G | p.Y1719C | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5215C > T | p.R1739X | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5227C > T | p.R1743W | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5309C > A | p.A1770D | RPE, Photoreceptors, Cochlear hair | Int 32 (NG 009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5392C > T | p.Q1798X | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5507T > C | p.L1836P | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5573T > C | p.L1858P | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5581C > T | p.R1861X | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5617C > T | p.R1873W | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells cells, Olfactory epithelium | | to target intron | p.Stop2216] (NM_000260.3) | |
| MYO7A | Usher | c.5618G > A | p.R1873Q | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5648G > A | p.R1883Q | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5660C > T | p.P1887L | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5686C > T | p.Q1896X | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5749G > T | p.E1917X | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5944G > A | p.G1982R | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5945G > A | p.G1982E | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5968C > T | p.Q1990X | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6028G > A | p.D2010N | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6043T > C | p.Y2015H | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6070C > T | p.R2024X | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6410G > A | p.G2137E | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6487G > A | p.G2163S | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6557T > C | p.L2186P | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6560G > A | p.G2187D | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6610G > C | p.A2204P | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.4502_4503delTG | p.Val1501Glysfs*2 | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.4648_4852 + 668del | p.Pro1550Glnfs*27 | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.4770dup | p.Arg1591Serfs*2 | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.4838delA | p.Asp1613Valfs*32 | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.4919delG | p.Gly1640Alafs*5 | RPE, Photoreceptors, Cochlear hair | Int 32 (NG_009086.1) | ~30-250 nts complementary | Ex 33-49 [c.4324G p.Gly1442] through [c.6648A3′ | 3′ |
| | Syndrome | | | cells, Olfactory epithelium | | to target intron | p.Stop2216] (NM_000260.3) | |
| MYO7A | Usher | c.5004C > G | p.Tyr1668* | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5146_5148delGAG | p.Glu1716del | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5227C > T | p.Arg1743Trp | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5411delT | p.Leu1804Argfs*6 | RPE, Photoreceptors, Cochlear hair | Int 32 (NG_009086.1) | ~30-250 nts complementary | Ex 33-49 [c.4324G p.Gly1442] through [c.6648A3′ | 3′ |
| | Syndrome | | | cells, Olfactory epithelium | | to target intron | p.Stop2216] (NM_000260.3) | |
| MYO7A | Usher | c.5480 + 1G > A | p.? | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5502G > A | p.Trp1834* | RPE, Photoreceptors, Cochlear hair | Int 32 (NG_009086.1) | ~30-250 nts complementary | Ex 33-49 [c.4324G p.Gly1442] through [c.6648A3′ | 3′ |
| | Syndrome | | | cells, Olfactory epithelium | | to target intron | p.Stop2216] (NM_000260.3) | |
| MYO7A | Usher | c.5581C > T | p.Arg1861* | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5623C > T | p.Gln1875* | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5632delC | p.Leu1878* | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5637 − 1G > A | p.? | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5637 − 1G > T | p.? | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5750_*2614del | p.Ph1916_Lys2215del | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5824G > A | p.Fly1942* | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5835_5838swlCTTT | p.Phe1946Serfs*23 | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5824G > T | p.Gly1942* | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5835_5838delCTTT | p.Phe1946Serfs*23 | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5856G > A | p.Ala1915_lys1952del | RPE, Photoreceptors, Cochlear hair | Int 32 (NG_009086.1) | -30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5856 + 1G > A | p.? | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.5886_5888delCTT | p.Phe1963del | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6025delG | p.Ala2009Profs*32 | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6049C > T | p.Gln2017* | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6051 + 1G > A | p.? | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6070C > T | p.Arg2024* | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6193delC | p.Gln2066Argfs*36 | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6205_6206delAT | p.Ile2069Profs*6 | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6321G > A | p.Trp2107* | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6324_6339del | p.Thr2109Serfs*4 | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6354 + 628*+737del | p.Gln2119_Lys2215del | RPE, Photoreceptors, Cochlear hair | Int 32 (NG 009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6355_6645del | p.Gln2119_Lys2215del | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.6377delC | p.Pro2126Leufs*5 | RPE, Photoreceptors, Cochlear hair | Int 32 (NG_009086.1) | ~30-250 nts complementary | Ex 33-49 [c.4324G p.Gly1442] through [c.6648A | 3′ |
| | Syndrome | | | cells, Olfactory epithelium | | to target intron | p.Stop2216] (NM_000260.3) | |
| MYO7A | Usher | c.1007G > A | p.R336H | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1097T > C | p.L366P | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1132C > A | p.R378S | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1142C > T | p.T381M | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1190C > A | p.A397D | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1258A > T | p.K420X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1309G > A | p.D437N | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1325A > G | p.E442G | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1348G > C | p.E450Q | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1370C > T | p.A457V | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1373A > T | p.N458I | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1478A > C | p.Q493P | RPE, Photoreceptors, Cochlear hair | Int 18 (NG_009086.1) | ~30-250 nts complementary | Ex 1-18 [c.1A p.Met 1] through [c.2187G | 5′ |
| | Syndrome | | | cells, Olfactory epithelium | | to target intron | p.Lys729] (NM_000260.3) | |
| MYO7A | Usher | c.1508C > T | p.P503L | RPE, Photoreceptors, Cochlear hair | Int 18 (NG 009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1556G > A | p.G519D | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1591C > T | p.Q531X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1797G > A | p.M599I | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1884C > A | p.C628X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1900C > T | p.R634X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1945C > T | p.R649W | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1952T > C | p.L651P | RPE, Photoreceptors, Cochlear hair | Int 18 (NG_009086.1) | ~30-250 nts complementary | Ex 1-18 [c.1A p.Met1] through [c.2187G | 5 |
| | Syndrome | | | cells, Olfactory epithelium | | to target intron | p.Lys729] (NM_000260.3) | |
| MYO7A | Usher | c.1969C > T | p.R657W | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1996C > T | p.R666X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.199G > A | p.V67M | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2005C > T | p.R669X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2028C > G | p.Y676X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2164G > C | p.G722R | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2266C > T | p.R756W | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2302A > T | p.K768X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2323C > T | p.Q775X | RPE, Photoreceptors, Cochlear hair | Int 18 (NG_009086.1) | ~30-250 nts complementary | Ex 1-18 [c.1A p.Met1] through [c.2187G | 5 |
| | Syndrome | | | cells, Olfactory epithelium | | to target intron | p.Lys729] (NM_000260.3) | |
| MYO7A | Usher | c.2461C > T | p.Q821X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2476G > A | p.A826T | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2513G > A | p.W838X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.252C > G | p.N84K | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2557C > T | p.R853C | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.269G > C | p.R90P | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2863G > A | p.G955S | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2878G > T | p.E960X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2904G > T | p.E968D | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2914C > T | p.R972X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3134T > C | p.I1045T | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3171C > G | p.Y1057X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.318C > A | p.N106K | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3238A > T | p.K1080X | RPE, Photoreceptors, Cochlear hair | Int 18 (NG 009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3260T > C | p.L1087P | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3503G > C | p.R1168P | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3508G > A | p.E1170K | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3547C > A | p.P1183T | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3652G > A | p.G1218R | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3718C > T | p.R1240W | RPE, Photoreceptors, Cochlear hair | Int 18 (NG_009086.1) | ~30-250 nts complementary | Ex 1-18 [c.1A p.Met1] through [c.2187G | 5 |
| | Syndrome | | | cells, Olfactory epithelium | | to target intron | p.Lys729] (NM_000260.3) | |
| MYO7A | Usher | c.3719G > A | p.R1240Q | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3731C > G | p.P1244R | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3862G > C | p.A1288P | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.395C > T | p.P132L | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3979G > A | p.E1327K | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.397C > G | p.H133D | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4018G > C | p.A1340P | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.401T > A | p.I134N | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4029G > C | p.R1343S | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4045G > A | p.E1349K | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4117C > T | p.R1373X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.448C > T | p.R150X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.470G > A | p.S157N | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.47T > A | p.L16X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.47T > C | p.L16S | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.487G > A | p.G163R | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.487G > C | p.G163R | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.491A > G | p.K164R | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.494C > T | p.T165M | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.52C > T | p.Q18X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.592G > A | p.A198T | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.610A > G | p.T204A | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.616C > T | p.R206C | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.617G > A | p.R206H | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.629C > G | p.S210X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.634C > T | p.R212C | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.635G > A | p.R212H | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.640G > A | p.G214R | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.689C > T | p.A230V | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.700C > T | p.Q234X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.721C > A | p.R241S | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.721C > G | p.R241G | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.721C > T | p.R241C | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.722G > A | p.R241H | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.731G > C | p.R244P | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.73G > A | p.G25R | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.755A > G | p.Y252C | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.77C > A | p.A26E | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.905G > A | p.R302H | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.93C > A | p.C31X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.940G > T | p.E314X | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.977T > A | p.L326Q | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.999T > G | p.Y333X | RPE, Photoreceptors, Cochlear hair | Int 18 (NG_009086.1) | ~30-250 nts complementary | Ex 1-18 [c.1A p.Met 1] through [c.2187G | 5′ |
| | Syndrome | | | cells, Olfactory epithelium | | to target intron | p.Lys729] (NM_000260.3) | |
| MYO7A | Usher | c.−272 − ?_5168 + | p.? | RPE, Photoreceptors, Cochlear hair | Int 18 (NG_009086.1) | ~30-250 nts complementary | Ex 1-18 [c.1A p.Met1] through [c.2187G | 5′ |
| | Syndrome | 213del | | cells, Olfactory epithelium | | to target intron | p.Lys 729] (NM_000260.3) | |
| MYO7A | Usher | c.−46 − 2A > G | p.? | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.6_9dup | p.Leu4Aspfs*39 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.19 − 2A > G | p.? | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.33G > A | p.Trp11* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.54G > C | p.Gln18His | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.133 − 2A > G | Ip.? | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.223delG | p.Asp75Thrfs*31 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.223G > C | p.Asn84Lys | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.338_348dup | p.Glu117Serfs*33 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.358delC | p.Arg120Alafs*26 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.360delC | p.Gln121Serfs*25 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.397dup | p.His133Profs*7 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.397dupC | p.His133Profs*7 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.462C > A | p.Cys154* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.471 − 1G > A | p.? | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.490A > T | p.Lys164fs* | RPE, Photoreceptors, Cochlear hair | Int 18 (NG_009086.1) | ~30-250 nts complementary | Ex 1-18 [c.1A p.Met 1] through [c.2187G | 5′ |
| | Syndrome | | | cells, Olfactory epithelium | | to target intron | p.Lys729] (NM_000260.3) | |
| MYO7A | Usher | c.496delG | p.Glu166Argfs*5 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.581_582delCC | p.Pro194Hisfs*14 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.652_657del | p.Asp218_Ile219del | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.722G > C | p.Arg241Pro | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.726delC | p.Cys243Valfs*20 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.834C > A | p.Tyr278* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.938delC | p.Glu314Argfs*48 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.986dup | p.Asn330Glnfs*5 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1005_1012del | p.Arg336* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.115_ 1158delTG | p.Leu386Glnfs*56 | RPE, Photoreceptors, Cochlear hair | Int 18 (NG_009086.1) | ~30-250 nts complementary | Ex 1-18 [c.1A p.Met 1] through [c.2187G | 5′ |
| | Syndrome | | | cells, Olfactory epithelium | | to target intron | p.Lys729] (NM_000260.3) | |
| MYO7A | Usher | c.1303delC | p.Leu435Serfs*12 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1343 + 1G > A | p.? | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1344 − 2A > G | p.? | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1454delT | p.Leu485Argfs*14 | RPE, Photoreceptors, Cochlear hair | Int 18 (NG 009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1477C > T | p.Gln493* | RPE, Photoreceptors, Cochlear hair | Int 18 (NG_009086.1) | ~30-250 nts complementary | Ex 1-18 [c.1A p.Met 1] through [c.2187G | 5′ |
| | Syndrome | | | cells, Olfactory epithelium | | to target intron | p.Lys729] (NM_000260.3) | |
| MYO7A | Usher | c.1555 − 1G > C | p.? | RPE, Photoreceptors, Cochlear hair | Int 18 (NG_009086.1) | -30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1563delC | p.Asp521Glufs*8 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1591C > T | p.Gln531* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1595delA | p.His532Profs*90 | RPE, Photoreceptors, Cochlear hair | Int 18 (NG_009086.1) | -30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1623dup | p.Lys542Glnfs*5 | RPE, Photoreceptors, Cochlear hair | Int 18 (NG_009086.1) | ~30-250 nts complementary | Ex 1-18 [c.1A p.Met 1] through [c.2187G | 5′ |
| | Syndrome | | | cells, Olfactory epithelium | | to target intron | p.Lys729] (NM_000260.3) | |
| MYO7A | Usher | c.1690 + 1G > A | p.? | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1708C > T | p.Arg570* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1797G > A | p.Met599Ile | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1884C > A | p.Cys628* | RPE, Photoreceptors, Cochlear hair | Int 18 (NG_009086.1) | -30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1900C > T | p.Arg634* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1935G > A | p.Met645Ile | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.1935 + 1G > A | p.? | RPE, Photoreceptors, Cochlear hair | Int 18 (NG_009086.1) | ~30-250 nts complementary | Ex 1-18 [c.1A p.Met1] through [c.2187G | 5′ |
| | | | | cells, Olfactory epithelium | | to target intron | p.Lys729] (NM_000260.3) | |
| MYO7A | Usher | c.1952_1953insAG | p.Cys652Glyfs*11 | RPE, Photoreceptors, Cochlear hair | Int 18 (NG_009086.1) | ~30-250 nts complementary | Ex 1-18 [c.1A p.Met1] through [c.2187G | 5′ |
| | | | | cells, Olfactory epithelium | | to target intron | p.Lys729] (NM_000260.3) | |
| MYO7A | Usher | c.1954delT | p.Cys652Alafs*10 | RPE, Photoreceptors, Cochlear hair | Int 18 (NG_009086.1) | ~30-250 nts complementary | Ex 1-18 [c.1A p.Met 1] through [c.2187G | 5′ |
| | Syndrome | | | cells, Olfactory epithelium | | to target intron | p.Lys729] (NM_000260.3) | |
| MYO7A | Usher | c.2115C > A | p.Cys705* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2187 + 1G > A | p.? | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2241_2242delAG | p.Arg747Serfs*16 | RPE, Photoreceptors, Cochlear hair | Int 18 (NG 009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2283 − 1G > T | p.Ser762Cysfs*61 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2307delC | p.Asn769Lysfs*61 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2425delC | p.Gln809Serfs*42 | RPE, Photoreceptors, Cochlear hair | Int 18 (NG_009086.1) | ~30-250 nts complementary | Ex 1-18 [c.1A p.Met 1] through [c.2187G | 5′ |
| | Syndrome | | | cells, Olfactory epithelium | | to target intron | p.Lys729] (NM_000260.3) | |
| MYO7A | Usher | c.2443C > T | p.Gln821* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2500delC | p.Arg834Alafs*17 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2513G > A | p.Trp838* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2557C > T | p.Arg853Cys | RPE, Photoreceptors, Cochlear hair | Int 18 (NG 009086.1) | ~30-250 nts complementary | Ex 1-18 [c.1A p.Met 1] through [c.2187G | 5′ |
| | Syndrome | | | cells, Olfactory epithelium | | to target intron | p.Lys729] (NM_000260.3) | |
| MYO7A | Usher | c.2656_2664del | p.Ala886_Lys888del | RPE, Photoreceptors, Cochlear hair | Int 18 (NG_009086.1) | ~30-250 nts complementary | Ex 1-18 [c.1A p.Met 1] through [c.2187G | 5′ |
| | Syndrome | | | cells, Olfactory epithelium | | to target intron | p.Lys729] (NM_000260.3) | |
| MYO7A | Usher | c.2662dup | p.Ala889Glyfs*19 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2766_2779del | p.Lys923Alafs*8 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2797delC | p.Arg933Alafs*129 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.2874_ | p.Gln959Glysfs*5 | RPE, Photoreceptors, Cochlear hair | Int 18 (NG_009086.1) | ~30-250 nts complementary | Ex 1-18 [c.1A p.Met 1] through [c.2187G | 5′ |
| | Syndrome | 2878delCCAGG | | cells, Olfactory epithelium | | to target intron | p.Lys729] (NM_000260.3) | |
| MYO7A | Usher | c.3108 + 1G > A | p.? | RPE, Photoreceptors, Cochlear hair | Int 18 (NG 009086.1) | -30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3135dup | p.Leu1046Profs*9 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3238A > T | p.Lys1080* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3260T > C | p.Leu1087Pro | RPE, Photoreceptors, Cochlear hair | Int 18 (NG 009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3262C > T | p.Gln1088* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3285delG | p.Ala1089Profs*19 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3504 − 1G > C | p.? | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3594C > A | p.Cys1198* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3596dup | p.Cys1201Leufs*28 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3631delT | p.Tyr1211Thrfs*21 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3702delC | p.Phe1235Leufs*28 | RPE, Photoreceptors, Cochlear hair | Int 18 (NG 009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3719G > A | p.Arg1240G1 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3724C > T | p.Gln1242* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3750 + 2T > A | p.? | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.3764delA | p.Lys1255Argfs*97 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4012delC | p.Arg1338Alafs*61 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4036_4038delTTC | p.Phe1346del | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4039_4053del | p.Arg1347_Phe1351del | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4045G > A | p.Glu1349Lys | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4065delC | p.His1355Glnfs*44 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4117C > T | p.Arg1373* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4131dup | p.Gly1378Trpfs*6 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4166delC | p.Ala1389Valfs*10 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4293G > A | p.Trp1431* | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4297delC | p.Gln1433Serfs*116 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |
| MYO7A | Usher | c.4483_4484dup | p.Trp1495Cysfs*55 | RPE, Photoreceptors, Cochlear hair | Int 18 (NG_009086.1) | ~30-250 nts complementary | Ex 1-18 [c.1A p.Met1] through [c.2187G | 5′ |
| | Syndrome | | | cells, Olfactory epithelium | | to target intron | p.Lys729] (NM_000260.3) | |

### The Pharmaceutical Carrier and Pharmaceutical Compositions

The compositions described herein containing the recombinant viral vector, e.g., AAV, containing the desired RTM minigene for use in the selected target ocular cells, e.g., photoreceptor cells for treatment of Stargardt Disease, as detailed above, is preferably assessed for contamination by conventional methods and then formulated into a pharmaceutical composition intended for a suitable route of administration. Still other compositions containing the RTM, e.g., naked DNA or as protein, may be formulated similarly with a suitable carrier. Such formulation involves the use of a pharmaceutically and/or physiologically acceptable vehicle or carrier, particularly directed for administration to the target cell. In one embodiment, carriers suitable for administration to the cells of the eye include buffered saline, an isotonic sodium chloride solution, or other buffers, e.g., HEPES, to maintain pH at appropriate physiological levels, and, optionally, other medicinal agents, pharmaceutical agents, stabilizing agents, buffers, carriers, adjuvants, diluents, etc.

For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20.

In other embodiments, e.g., compositions containing RTMs described herein include a surfactant. Useful surfactants, such as Pluronic F68 ((Poloxamer 188), also known as Lutrol®) F68) may be included as they prevent AAV from sticking to inert surfaces and thus ensure delivery of the desired dose.

As an example, one illustrative composition designed for the treatment of the ocular diseases described herein comprises a recombinant adeno-associated vector carrying a nucleic acid sequence encoding 3′RTM as described herein, tinder the control of regulatory sequences which express the RTM in an ocular cell of a mammalian subject, and a pharmaceutically acceptable carrier. The carrier is isotonic sodium chloride solution and includes a surfactant Pluronic F68. In one embodiment, the RTM is that described in the examples. In another embodiment, the RTM contains the binding and coding regions for CEP290 or MYO7A.

In yet another exemplary embodiment, the composition comprises a recombinant AAV2/5 pseudotyped adeno-associated virus carrying a 3′ or 5′ or RTM for internal ocular gene replacement, the nucleic acid sequence under the control of promoter which directs expression of the RTM in said photoreceptor cells, wherein the composition is formulated with a carrier and additional components suitable for subretinal injection. In still another embodiment, the composition or components for production or assembly of this composition, including carriers, rAAV particles, surfactants, and/or the components for generating the rAAV, as well as suitable laboratory hardware to prepare the composition, may be incorporated into a kit.

### Methods of Treating Ocular Disorders

The compositions described above are thus useful in methods of treating one or more of the ocular diseases (e.g., Stargardt Disease, Lebers Congenital Amaurosis, cone rod dystrophy, fundus flavimaculatus, retinitis pigmentosa, age-related macular degeneration, Senior L\tilde{ø}ken syndrome, Joubert syndrome, or Usher Syndrome, among others) including delaying or ameliorating symptoms associated with the ocular diseases described herein. Such methods involve contacting a target pre-mRNA (e.g., ABCA4, CEP290, MYO7A) with one or more of a 3′RTM, 5′ RTM, both 3′ and 5′ RTM or a double trans-splicing RTM as described herein, under conditions in which a portion of the RTM is spliced to the target pre-mRNA to replace all or a part of the targeted gene carrying one or more defects or mutations, with a “healthy”, or normal or wildtype or corrected mRNA of the targeted gene, in order to correct expression of that gene in the ocular cell. Alternatively, a pre-miRNA (see the RTM documents cited herein) can be formed, which is designed to reduce the expression of a target mRNA. Thus, the methods and compositions are used to treat the ocular diseases/pathologies associated with the specific mutations and/or gene expression.

In one embodiment, the contacting involves direct administration to the affected subject; in another embodiment, the contacting may occur ex vivo to the cultured cell and the treated ocular cell reimplanted in the subject. In one embodiment, the method involves administering a rAAV particle carrying a 3′ RTM. In another embodiment, the method involves administering a rAAV particle carrying a 5′ RTM. In another embodiment, the method involves administering a rAAV particle carrying a double trans-splicing RTM. In still another embodiment, the method involves administering a mixture of rAAV particle carrying a 3′ RTM and rAAV particle carrying a 5′ RTM. In still another embodiment, the method involves administering a mixture of rAAV particle carrying a 3′ RTM and an rAAV particle carrying a double trans-splicing RTM. In still another embodiment, the method involves administering a mixture of rAAV particle carrying a 5′ RTM and an rAAV carrying a double trans-splicing RTM. In still another embodiment, the method involves administering a mixture of an rAAV particle carrying a 3′ RTM, with an rAAV particle carrying a 5′ RTM and an rAAV particle carrying a double trans-splicing RTM.

These methods comprise administering to a subject in need thereof subject an effective concentration of a composition of any of those described herein.

In one illustrative embodiment, such a method is provided for preventing, arresting progression of or ameliorating vision loss associated with Stargardt Disease in a subject, said method comprising administering to an ocular cell of a mammalian subject in need thereof an effective concentration of a composition comprising a recombinant adeno-associated virus (AAV) carrying a 3′RTM such as described above and in the examples, under the control of regulatory sequences which permit the RTM to function and cause trans-splicing of the defective targeted gene in an ocular cell, e.g., photoreceptor cell, of a mammalian subject. In still another embodiment, the method involves administering two rAAV particles, one carrying a 5′ RTM and the other carrying the 3′RTM, such as those RTMs described in the examples to replace large portions of large genes.

By “administering” as used in the methods means delivering the composition to the target selected cell which is characterized by the disease caused by a mutation or defect in the targeted ocular gene. For example, in one embodiment, the method involves delivering the composition by subretinal injection to the photoreceptor cells or other ocular cells. In another embodiment, intravitreal injection to ocular cells or injection via the palpebral vein to ocular cells may be employed. Still other methods of administration may be selected by one of skill in the art given this disclosure.

Furthermore, in certain embodiments, it is desirable to perform non-invasive retinal imaging and functional studies to identify areas of retained photoreceptors to be targeted for therapy. In these embodiments, clinical diagnostic tests are employed to determine the precise location(s) for one or more subretinal injection(s). These tests may include electroretinography (ERG), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc. In view of the imaging and functional studies, in some embodiments one or more injections are performed in the same eye in order to target different areas of retained photoreceptors.

For use in these methods, the volume and viral titer of each injection is determined individually, as further described below, and may be the same or different from other injections performed in the same, or contralateral, eye. In another embodiment, a single, larger volume injection is made in order to treat the entire eye. The dosages, administrations and regimens may be determined by the attending physician given the teachings of this specification.

In one embodiment, the volume and concentration of the rAAV composition is selected so that only the certain regions of photoreceptors or other ocular cell is impacted. In another embodiment, the volume and/or concentration of the rAAV composition is a greater amount, in order reach larger portions of the eye. Similarly dosages are adjusted for administration to other organs.

An effective concentration of a recombinant adeno-associated virus carrying a RTM as described herein ranges between about 10⁸ and 10¹³ vector genomes per milliliter (vg/mL). The rAAV infectious units are measured as described in S. K. McLaughlin et al, 1988 J. Virol., 62:1963. In another embodiment, the concentration ranges between 109 and 10¹³ vector genomes per milliliter (vg/mL). In another embodiment, the effective concentration is about 1.5×10¹¹ vg/mL. In one embodiment, the effective concentration is about 1.5×10¹⁰ vg/mL. In another embodiment, the effective concentration is about 2.8×10¹¹ vg/mL. In yet another embodiment, the effective concentration is about 1.5×10¹² vg/mL. In another embodiment, the effective concentration is about 1.5×10¹³ vg/mL. It is desirable that the lowest effective concentration of virus be utilized in order to reduce the risk of undesirable effects, such as toxicity, and other issues related to administration to the eye, e.g., retinal dysplasia and detachment. Still other dosages in these ranges or in other units may be selected by the attending physician, taking into account the physical state of the subject, preferably human, being treated, including the age of the subject; the composition being administered and the particular ocular disorder; the targeted cell and the degree to which the disorder, if progressive, has developed.

The composition may be delivered in a volume of from about 50 μL to about 1 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 IL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL.

In another embodiment, the invention provides a method to prevent, or arrest photoreceptor function loss, or increase photoreceptor function in the subject. The composition may be administered before disease onset or after initiation of photoreceptor loss. Photoreceptor function may be assessed using the functional studies, e.g., ERG or perimetry, which are conventional in the art. As used herein “photoreceptor function loss” means a decrease in photoreceptor function as compared to anormal, non-diseased eye or the same eye at an earlier time point. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same patient.

For each of the described methods, the treatment may be used to prevent the occurrence of further damage or to rescue tissues or organ, e.g., eyes in a subject with LCA10 or Stargardt Disease or Ushers Syndrome or retinitis pigmentosa, having mild or advanced disease. As used herein, the term “rescue” means to prevent progression of the disease, prevent spread of damage to uninjured ocular cells or to improve damage in injured ocular cells.

Thus, in one embodiment, the composition is administered before disease onset. In another embodiment, the composition is administered prior to the initiation of vision impairment or loss. In another embodiment, the composition is administered after initiation of vision impairment or loss. In yet another embodiment, the composition is administered when less than 90% of the photoreceptors are functioning or remaining, as compared to a non-diseased eye.

In another embodiment, the method includes performing functional and imaging studies to determine the efficacy of the treatment. These studies include ERG and in vivo retinal imaging, as described in U.S. Pat. No. 8,147,823; in co-pending International patent application publication WO 2014/011210 or WO 2014/124282, incorporated by reference. In addition visual field studies, perimetry and microperimetry, mobility testing, visual acuity, color vision testing may be performed.

In yet another embodiment, any of the above described methods is performed in combination with another, or secondary, therapy. The therapy may be any now known, or as yet unknown, therapy which helps prevent, arrest or ameliorate these mutations or defects or any of the effects associated therewith. The secondary therapy can be administered before, concurrent with, or after administration of the rAAVs described above. In one embodiment, a secondary therapy involves non-specific approaches for maintaining the health of the retinal cells, such as administration of neurotrophic factors, anti-oxidants, anti-apoptotic agents. The non-specific approaches are achieved through injection of proteins, recombinant DNA, recombinant viral vectors, stem cells, fetal tissue, or genetically modified cells. The latter could include genetically modified cells that are encapsulated.

The compositions and methods described herein are believed to have many advantages over any currently employed therapies. Firstly, the use of the RTM delivery by rAAV provides efficient and specific delivery of a gene therapy to photoreceptors. Secondly, these compositions and methods permit correction of the genetic defect at the source. Additionally, these compositions and methods provide are useful to treat any type of mutation in ABCA4 (or other large cDNAs/transgene cassettes). Correction of the defect in photoreceptors provides secondary rescue to retinal pigment epithelium cells. Further, the method of gene correction is benign immunologically. As there is currently no other treatment available for ABCA4-mediated disease (or other retinal disease caused by defects in transgenes with large cDNAs, these methods and compositions are clearly valuable. The use of subretinal delivery and other features renders the effect specific to photoreceptors, so that toxicity due to off-target splicing is likely minimal. Finally, RNA repair does not require cell division, whereas DNA repair methodologies (such as CRISPR-Cas9 or zinc fingers) have a requirement for the cell to go through mitosis for homology directed repair to occur, which is a disadvantage in post-mitotic tissues like the retina.

Restoration of cellular function by the method described herein can be assessed in an animal model of the appropriate disease caused by defect or mutation, such as the restoration of visual function in a subject with a CEP290 defect causing LCA in the rd16 mouse LCA model or canine model of LCA. The use of the exemplary rAAV carrying an RTM as described herein can demonstrate that the defect in the mutant dog or other animal model could be corrected by gene delivery. This data allow one of skill in the art to readily anticipate that this method may be similarly used in treatment of other types of retinal disease in other subjects, including humans.

The examples that follow do not limit the scope of the embodiments described herein. One skilled in the art will appreciate that modifications can be made in the following examples which are intended to be encompassed by the spirit and scope of the invention.

### Example 1: Splicing Dependent Reporter RTM

A splicing dependent reporter RTM is a molecule comprising a binding domain, spacer, and 3′ splice site. The binding domain can be selected from appropriate binding domains for the selected targeted intron, and the 3′ splice site can be any of those disclosed herein. A trans-splicing dependent reporter RTM contains the complete coding DNA sequence of green fluorescent protein, but lacking the first three bases, ATG, constituting the start codon. The molecule does not have an open reading frame for GFP. Therefore, GFP is only translated if it is spliced in-frame and 3′ to a trans-pre-mRNA. These reagents split the complete coding DNA sequence between two plasmids to reconstitute GFP via trans-splicing. This is a novel reagent with potential commercial use for evaluating the occurrence of trans-splicing with a single plasmid.

### Example 2: rAAV—RTM Assembly for Stargardt's Disease

For the structures of the 3′ RTM or 5′ RTM for ABCA4, see FIG. 1.

A 3′ RTM is designed with a binding domain that targets intron 26 (4,696 bp NG 009073.1). The 3′ RTM molecule for the ABCA4 trans-splicing comprises:
3′ RTM promoter
3′ RTM Binding domain sequence: 70-2000 nucleotides complementary to target intron 26;
3′ RTM Spacer sequence: GAGAACATTATTATAGCGTTGCTCGAG SEQ ID NO: 10
3′ RTM Branch point sequence: TACTAAC;
3′ RTM Polypyrimidine tract: TGGTACCTCTTCTTTJTTTTCTG SEQ ID NO: 11
3′ acceptor splice site: CAGGT;
Coding domain of 2,930 bp ABCA4 cDNA encoding exons 27 through the terminal exon 50; and
3′RTM polyA signal sequence.

In another embodiment a 5′ RTM molecule for the ABCA4 trans-splicing comprises:
3′ RTM promoter:
5′RTM coding domain of 3,328 bp ABCA4 cDNA encoding exons 1 through the exon 22;
5′ RTM 5′ Splice Site: AGGT;
5′RTM spacer sequence: AGAGCTCGTTGCGATATTAT SEQ ID NO: 12; Binding domain sequence: 70-2000 nucleotides complementary to target intron 22 (1,358 bp NG 009073.1); and
5′ RTM PolyA sequence.

The pair of trans-splicing reagents covers mutations spaced over the entire coding ABCA4 coding sequence. The two cDNA molecules are derived from a mammalian codon optimized sequence of ABCA4.

Each RTM is introduced into a proviral plasmid p618 as referenced above, following the teachings of WO2012/158757. The proviral plasmids are cultured in the host cells which express the cap and/or rep proteins. In the host cells, each minigene consisting of the RTM with flanking AA ITRs is rescued and packaged into the capsid protein or envelope protein to form an infectious viral particle. Thus two types of recombinant AAV infectious particle are produced and purified from culture: one carrying the 3′RTM and the other carrying the 5′RTM. See, e.g., FIGS. 3A and 3B and TABLE 4, which is the sequence of the RTM of FIG. 3A in GenBank format which delineates features of the sequence.

TABLE 4
pAAV ABCA4 3′RTM CMV CMB chimInt BD_I26 3′SS Ex 27_50
synthetic DNA construct-12513 bp ds-DNA circular recombinant plasmid
REFERENCE 1 (bases 1 to 12513) SEQ ID NO: 1

| Features | Location/Qualifiers |
|---|---|
| Source | 1..12513 |
| | /organism = ″recombinant plasmid″ |
| | /mol_type = ″other DNA″ |
| Repeat Region | 1..130 |
| | /note = ″5′ ITR″ |
| Misc Feature | 113..130 |
| | /note = ″ITR D segment″ |
| Enhancer | 241..544 |
| | /note = ″CMV enhancer″ |
| | /note = ″human cytomegalovirus immediate early enhancer″ |
| Promoter | 546..823 |
| | /note = ″chicken beta-actin promoter″ |
| Intron | 919..1051 |
| | /note = ″chimeric intron″ |
| | /note = ″chimera between introns from human beta-globin and |
| | immunoglobulin heavy chain genes″ |
| Intron | complement (1074..1222) |
| | /label = ″Intron 26″ |
| | /note = ″ABCA4 I26 BD″ |
| misc_feature | 1243..1269 |
| | /gene = ″spacer″ |
| | /note = ″spacer″ |
| misc_feature | 1270..1297 |
| | /note = ″3′SS″ |
| gene | 1298..4257 |
| | /label = ″ABCA4 NM_000350.2″ |
| misc_feature | 1298..1563 |
| | /label = ″Exon 27″ |
| | /note = ″Exon 27″ |
| misc_feature | 1564..1688 |
| | /label = ″Exon 28″ |
| | /note = ″Exon 28″ |
| misc_feature | 1689..1787 |
| | /label = ″Exon 29″ |
| | /label = ″Exon 29″ |
| | /note = ″Exon 29″ |
| misc_feature | 1788..1974 |
| | /label = ″Exon 30″ |
| | /note = ″Exon 30″ |
| misc_feature | 1975..2069 |
| | /label = ″Exon 31″ |
| | /note = ″Exon 31″ |
| misc_feature | 2070..2102 |
| | /label = ″Exon 32″ |
| | /note = ″Exon 32″ |
| misc_feature | 2103..2208 |
| | /label = ″Exon 33″ |
| | /note = ″Exon 33″ |
| misc_feature | 2209..2283 |
| | /label = ″Exon 34″ |
| | /note = ″Exon 34″ |
| misc_feature | 2284..2453 |
| | /label = ″Exon 35″ |
| | /note = ″Exon 35″ |
| misc_feature | 2454..2631 |
| | /label = ″Exon 36″ |
| | /note = ″Exon 36″ |
| misc_feature | 2632..2747 |
| | /label = ″Exon 37″ |
| | /note = ″Exon 37″ |
| misc_feature | 2748..2895 |
| | /label = ″Exon 38″ |
| | /note = ″Exon 38″ |
| misc_feature | 2896..3019 |
| | /label = ″Exon 39″ |
| | /note = ″Exon 39″ |
| misc_feature | 3020..3149 |
| | /label = ″Exon 40″ |
| | /note = ″Exon 40″ |
| misc_feature | 3150..3270 |
| | /label = ″Exon 41″ |
| | /note = ″Exon 41″ |
| misc_feature | 3271..3333 |
| | /label = ″Exon 42″ |
| | /note = ″Exon 42″ |
| misc_feature | 3334..3440 |
| | /label = ″Exon 43″ |
| | /note = ″Exon 43″ |
| misc_feature | 3441..3582 |
| | /label = ″Exon 44″ |
| | /note = ″Exon 44″ |
| misc_feature | 3583..3717 |
| | /label = ″Exon 45″ |
| | /note = ″Exon 45″ |
| misc_feature | 3718..3821 |
| | /label = ″Exon 46″ |
| | /note = ″Exon 46″ |
| misc_feature | 3822..3914 |
| | /label = ″Exon 47″ |
| | /note = ″Exon 47″ |
| misc_feature | 3915..4164 |
| | /label = ″Exon 48″ |
| | /note = ″Exon 48″ |
| misc_feature | 4165..4251 |
| | /label = ″Exon 49″ |
| | /note = ″Exon 49″ |
| misc_feature | 4252..4257 |
| | /label = ″Exon 50″ |
| | /note = ″Exon 50″ |
| polyA_signal | 4275..4482 |
| | /note = ″bGH poly(A) signal″ |
| | /note = ″bovine growth hormone polyadenylation signal″ |
| Repeat region | 4532..4661 |
| | /note = ″3′ ITR″ |
| misc_feature | 4532..4549 |
| | /note = ″ITR D segment″ |
| protein_bind | complement (4689..4722) |
| | /bound_moiety = ″FLP recombinase from the Saccharomyces |
| | cerevisiae 2u plasmid″ |
| | /note = ″FRT (minimal)″ |
| | /note = ″supports FLP-mediated excision but not integration |
| | (Turan and Bode, 2011)″ |
| misc_feature | 4755..5055 |
| | /product = ″bla txn terminator″ |
| | /note = ″bla txn terminator″ |
| misc_feature | 4846..4871 |
| | /product = ″pTF3″ |
| | /note = ″pTF3″ |
| misc_feature | 5062..5175 |
| | /product = ″rpn txn terminator″ |
| | /note = ″rpn txn terminator″ |
| misc_feature | 5191..10257 |
| | /note = ″lambda stuffer″ |
| primer_bind | complement (10263..10279) |
| | /note = ″M13 fwd″ |
| | /note = ″common sequencing primer, one of multiple similar |
| | variants″ |
| rep_origin | complement (10549..11137) |
| | /direction = LEFT |
| | /note = ″ori″ |
| | /note = ″high-copy-number ColE1/pMB1/pBR322/pUC origin of |
| | replication″ |
| CDS SEQ ID | Complement (11261..12070) |
| NO: 3 | /codon_start = 1 |
| | /gene = ″aph(3′)-Ia″ |
| | /product = ″aminoglycoside phosphotransferase″ |
| | /note = ″KanR″ |
| | /note = ″confers resistance to kanamycin in bacteria or G418 |
| | (Geneticin(R)) in eukaryotes″ |
| | /translation = ″MSHIQRETSRPRLNSNMDADLYGYKWARDNVGQSGATIYRLYGKPDA |
| | PELFLKHGKGSVANDVTDEMVRLNWLTEF |
| | MPLPTIKHFIRTPDDAWLLTTAIPGKTAFQVLEEYPDSGE |
| | NIVDALAVFLRRLHSIPVCNCPFNSDRVFRLAQAQSRMN |
| | NGLVDASDFDDERNGWPVEQVWKEMHKLLPFSPDSVVT |
| | HGDFSLDNLIFDEGKLIGCIDVGRVGIADRYQDLAILWNCL |
| | GEFSPSLQKRLFQKYGIDNPDMNKLQFHLMLDEFF″ |
| promoter | complement(12071..12162) |
| | /gene = ″bla″ |
| | /note = ″AmpR promoter″ |
| misc_feature | complement(12249..12423) |
| | /product = ″rrnB1 B T1 txn terminator″ |
| | /note = ″rrnB1 B2 T1 txn terminator″ |
| misc_feature | 12324..12340 |
| | /product = ″pTR″ |
| | /note = ″pTR″ |
| protein_bind | 12455..12488 |
| | /bound_moiety = ″FLP recombinase from the Saccharomyces |
| | cerevisiae 2u plasmid″ |
| | /note = ″FRT (minimal)″ |
| | /note = ″supports FLP-mediated excision but not integration |
| | (Turan and Bode, 2011)″ |

ORIGIN SEQ ID NO: 1

| | |
|---|---|
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct tgtagttaat gattaacccg ccatgctact tatctacgta gcaagctagc |
| 181 | tagttattaa tagtaatcaa ttacggggtc attagttcat agcccatata tggagttccg |
| 241 | cgttacataa cttacggtaa atggcccgcc tggctgaccg cccaacgacc cccgcccatt |
| 301 | gacgtcaata atgacgtatg ttcccatagt aacgccaata gggactttcc attgacgtca |
| 361 | atgggtggag tatttacggt aaactgccca cttggcagta catcaagtgt atcatatgcc |
| 421 | aagtacgccc cctattgacg tcaatgacgg taaatggccc gcctggcatt atgcccagta |
| 481 | catgacctta tgggactttc ctacttggca gtacatctac gtattagtca tcgctattaa |
| 541 | catggtcgag gtgagcccca cgttctgctt cactctcccc atctcccccc cctccccacc |
| 601 | cccaattttg tatttattta ttttttaatt attttgtgca gcgatggggg cggggggggg |
| 661 | gggggggcgc gcgccaggcg gggcggggcg gggcgagggg cggggcgggg cgaggcggag |
| 721 | aggtgcggcg gcagccaatc agagcggcgc gctccgaaag tttcctttta tggcgaggcg |
| 781 | gcggcggcgg cggccctata aaaagcgaag cgcgcggcgg gcggggagtc gctgcgacgc |
| 841 | tgccttcgcc ccgtgccccg ctccgccgcc gcctcgcgcc gcccgccccg gctctgactg |
| 901 | accgcgttac tcccacaggt aagtatcaag gttacaagac aggtttaagg agaccaatag |
| 961 | aaactgggct tgtcgagaca gagaagactc ttgcgtttct gataggcacc tattggtctt |
| 1021 | actgacatcc actttgcctt tctctccaca ggttggtgta cactagcggc cgcaaactct |
| 1081 | gctacactca cacatgcttt gtgtggctgt gggtttgata aaagttcatg gaaggagcta |
| 1141 | gttggtgccc aggctgacac atgtagaaga gagacttcta gaatccacag gaattttggt |
| 1201 | ccccatgttt tcaaagccca tacaagcttc gaattcgata tcgagaacat tattatagcg |
| 1261 | ttgctcgagt actaactggt acctcttctt ttttttcgtg gcgctcagca gaaaagagaa |
| 1321 | aacgtcaacc cccgacaccc ctgcttgggt cccagagaga aggctggaca gacaccccag |
| 1381 | gactccaatg tctgctcccc aggggcgccg gctgctcacc cagagggcca gcctccccca |
| 1441 | gagccagagt gcccaggccc gcagctcaac acggggacac agctggtcct ccagcatgtg |
| 1501 | caggcgctgc tggtcaagag attccaacac accatccgca gccacaagga cttcctggcg |
| 1561 | cagatcgtgc tcccggctac ctttgtgttt ttggctctga tgctttctat tgttatccct |
| 1621 | ccttttggcg aataccccgc tttgaccctt cacccctgga tatatgggca gcagtacacc |
| 1681 | ttcttcagca tggatgaacc aggcagtgag cagttcacgg tacttgcaga cgtcctcctg |
| 1741 | aataagccag gctttggcaa ccgctgcctg aaggaagggt ggcttccgga gtacccctgt |
| 1801 | ggcaactcaa caccctggaa gactccttct gtgtccccaa acatcaccca gctgttccag |
| 1861 | aagcagaaat ggacacaggt caacccttca ccatcctgca ggtgcagcac cagggagaag |
| 1921 | ctcaccatgc tgccagagtg ccccgagggt gccgggggcc tcccgccccc ccagagaaca |
| 1981 | cagcgcagca cggaaattct acaagacctg acggacagga acatctccga cttcttggta |
| 2041 | aaaacgtatc ctgctcttat aagaagcagc ttaaagagca aattctgggt caatgaacag |
| 2101 | aggtatggag gaatttccat tggaggaaag ctcccagtcg tccccatcac gggggaagca |
| 2161 | cttgttgggt ttttaagcga ccttggccgg atcatgaatg tgagcggggg ccctatcact |
| 2221 | agagaggcct ctaaagaaat acctgatttc cttaaacatc tagaaactga agacaacatt |
| 2281 | aaggtgtggt ttaataacaa aggctggcat gccctggtca gctttctcaa tgtggcccac |
| 2341 | aacgccatct tacgggccag cctgcctaag gacaggagcc ccgaggagta tggaatcacc |
| 2401 | gtcattagcc aacccctgaa cctgaccaag gagcagctct cagagattac agtgctgacc |
| 2461 | acttcagtgg atgctgtggt tgccatctgc gtgattttct ccatgtcctt cgtcccagcc |
| 2521 | agctttgtcc tttatttgat ccaggagcgg gtgaacaaat ccaagcacct ccagtttatc |
| 2581 | agtggagtga gccccaccac ctactgggtg accaacttcc tctgggacat catgaattat |
| 2641 | tccgtgagtg ctgggctggt ggtgggcatc ttcatcgggt ttcagaagaa agcctacact |
| 2701 | tctccagaaa accttcctgc ccttgtggca ctgctcctgc tgtatggatg ggcggtcatt |
| 2761 | cccatgatgt acccagcatc cttcctgttt gatgtcccca gcacagccta tgtggcttta |
| 2821 | tcttgtgcta atctgttcat cggcatcaac agcagtgcta ttaccttcat cttggaatta |
| 2881 | tttgagaata accggacgct gctcaggttc aacgccgtgc tgaggaagct gctcattgtc |
| 2941 | ttcccccact tctgcctggg ccggggcctc attgaccttg cactgagcca ggctgtgaca |
| 3001 | gatgtctatg cccggtttgg tgaggagcac tctgcaaatc cgttccactg ggacctgatt |
| 3061 | gggaagaacc tgtttgccat ggtggtggaa ggggtggtgt acttcctcct gaccctgctg |
| 3121 | gtccagcgcc acttcttcct ctcccaatgg attgccgagc ccactaagga gcccattgtt |
| 3181 | gatgaagatg atgatgtggc tgaagaaaga caaagaatta ttactggtgg aaataaaact |
| 3241 | gacatcttaa ggctacatga actaaccaag atttatccag gcacctccag cccagcagtg |
| 3301 | gacaggctgt gtgtcggagt tcgccctgga gagtgctttg gcctcctggg agtgaatggt |
| 3361 | gccggcaaaa caaccacatt caagatgctc actggggaca ccacagtgac ctcaggggat |
| 3421 | gccaccgtag caggcaagag tattttaacc aatatttctg aagtccatca aaatatgggc |
| 3481 | tactgtcctc agtttgatgc aattgatgag ctgctcacag gacgagaaca tctttacctt |
| 3541 | tatgcccggc ttcgaggtgt accagcagaa gaaatcgaaa aggttgcaaa ctggagtatt |
| 3601 | aagagcctgg gcctgactgt ctacgccgac tgcctggctg gcacgtacag tgggggcaac |
| 3661 | aagcggaaac tctccacagc catcgcactc attggctgcc caccgctggt gctgctggat |
| 3721 | gagcccacca cagggatgga cccccaggca cgccgcatgc tgtggaacgt catcgtgagc |
| 3781 | atcatcagag aagggagggc tgtggtcctc acatcccaca gcatggaaga atgtgaggca |
| 3841 | ctgtgtaccc ggctggccat catggtaaag ggcgcctttc gatgtatggg caccattcag |
| 3901 | catctcaagt ccaaatttgg agatggctat atcgtcacaa tgaagatcaa atccccgaag |
| 3961 | gacgacctgc ttcctgacct gaaccctgtg gagcagttct tccaggggaa cttcccaggc |
| 4021 | agtgtgcaga gggagaggca ctacaacatg ctccagttcc aggtctcctc ctcctccctg |
| 4081 | gcgaggatct tccagctcct cctctcccac aaggacagcc tgctcatcga ggagtactca |
| 4141 | gtcacacaga ccacactgga ccaggtgttt gtaaattttg ctaaacagca gactgaaagt |
| 4201 | catgacctcc ctctgcaccc tcgagctgct ggagccagtc gacaagccca ggactgactg |
| 4261 | cagatctgcc tcgactgtgc cttctagttg ccagccatct gttgtttgcc cctcccccgt |
| 4321 | gccttccttg accctggaag gtgccactcc cactgtcctt tcctaataaa atgaggaaat |
| 4381 | tgcatcgcat tgtctgagta ggtgtcattc tattctgggg ggtggggtgg ggcaggacag |
| 4441 | caagggggag gattgggaag acaatagcag gcatgctggg gactcgagtt ctacgtagat |
| 4501 | aagtagcatg gcgggttaat cattaactac aaggaacccc tagtgatgga gttggccact |
| 4561 | ccctctctgc gcgctcgctc gctcactgag gccgggcgac caaaggtcgc ccgacgcccg |
| 4621 | ggctttgccc gggcggcctc agtgagcgag cgagcgcgca gccttaatta acctaaggaa |
| 4681 | aatgaagtga agttcctata ctttctagag aataggaact tctatagtga gtcgaataag |
| 4741 | ggcgacacaa aatttattct aaatgcataa taaatactga taacatctta tagtttgtat |
| 4801 | tatattttgt attatcgttg acatgtataa ttttgatatc aaaaactgat tttcccttta |
| 4861 | ttattttcga gatttatttt cttaattctc tttaacaaac tagaaatatt gtatatacaa |
| 4921 | aaaatcataa ataatagatg aatagtttaa ttataggtgt tcatcaatcg aaaaagcaac |
| 4981 | gtatcttatt taaagtgcgt tgcttttttc tcatttataa ggttaaataa ttctcatata |
| 5041 | tcaagcaaag tgacaggcgc ccttaaatat tctgacaaat gctctttccc taaactcccc |
| 5101 | ccataaaaaa acccgccgaa gcgggttttt acgttatttg cggattaacg attactcgtt |
| 5161 | atcagaaccg cccagggggc ccgagcttaa cctttttatt tgggggagag ggaagtcatg |
| 5221 | aaaaaactaa cctttgaaat tcgatctcca gcacatcagc aaaacgctat tcacgcagta |
| 5281 | cagcaaatcc ttccagaccc aaccaaacca atcgtagtaa ccattcagga acgcaaccgc |
| 5341 | agcttagacc aaaacaggaa gctatgggcc tgcttaggtg acgtctctcg tcaggttgaa |
| 5401 | tggcatggtc gctggctgga tgcagaaagc tggaagtgtg tgtttaccgc agcattaaag |
| 5461 | cagcaggatg ttgttcctaa ccttgccggg aatggctttg tggtaatagg ccagtcaacc |
| 5521 | agcaggatgc gtgtaggcga atttgcggag ctattagagc ttatacaggc attcggtaca |
| 5581 | gagcgtggcg ttaagtggtc agacgaagcg agactggctc tggagtggaa agcgagatgg |
| 5641 | ggagacaggg ctgcatgata aatgtcgtta gtttctccgg tggcaggacg tcagcatatt |
| 5701 | tgctctggct aatggagcaa aagcgacggg caggtaaaga cgtgcattac gttttcatgg |
| 5761 | atacaggttg tgaacatcca atgacatatc ggtttgtcag ggaagttgtg aagttctggg |
| 5821 | atataccgct caccgtattg caggttgata tcaacccgga gcttggacag ccaaatggtt |
| 5881 | atacggtatg ggaaccaaag gatattcaga cgcgaatgcc tgttctgaag ccatttatcg |
| 5941 | atatggtaaa gaaatatggc actccatacg tcggcggcgc gttctgcact gacagattaa |
| 6001 | aactcgttcc cttcaccaaa tactgtgatg accatttcgg gcgagggaat tacaccacgt |
| 6061 | ggattggcat cagagctgat gaaccgaagc ggctaaagcc aaagcctgga atcagatatc |
| 6121 | ttgctgaact gtcagacttt gagaaggaag atatcctcgc atggtggaag caacaaccat |
| 6181 | tcgatttgca aataccggaa catctcggta actgcatatt ctgcattaaa aaatcaacgc |
| 6241 | aaaaaatcgg acttgcctgc aaagatgagg agggattgca gcgtgttttt aatgaggtca |
| 6301 | tcacgggatc ccatgtgcgt gacggacatc gggaaacgcc aaaggagatt atgtaccgag |
| 6361 | gaagaatgtc gctggacggt atcgcgaaaa tgtattcaga aaatgattat caagccctgt |
| 6421 | atcaggacat ggtacgagct aaaagattcg ataccggctc ttgttctgag tcatgcgaaa |
| 6481 | tatttggagg gcagcttgat ttcgacttcg ggagggaagc tgcatgatgc gatgttatcg |
| 6541 | gtgcggtgaa tgcaaagaag ataaccgctt ccgaccaaat caaccttact ggaatcgatg |
| 6601 | gtgtctccgg tgtgaaagaa caccaacagg ggtgttacca ctaccgcagg aaaaggagga |
| 6661 | cgtgtggcga gacagcgacg aagtatcacc gacataatct gcgaaaactg caaatacctt |
| 6721 | ccaacgaaac gcaccagaaa taaacccaag ccaatcccaa aagaatctga cgtaaaaacc |
| 6781 | ttcaactaca cggctcacct gtgggatatc cggtggctaa gacgtcgtgc gaggaaaaca |
| 6841 | aggtgattga ccaaaatcga agttacgaac aagaaagcgt cgagcgagct ttaacgtgcg |
| 6901 | ctaactgcgg tcagaagctg catgtgctgg aagttcacgt gtgtgagcac tgctgcgcag |
| 6961 | aactgatgag cgatccgaat agctcgatgc acgaggaaga agatgatggc taaaccagcg |
| 7021 | cgaagacgat gtaaaaacga tgaatgccgg gaatggtttc accctgcatt cgctaatcag |
| 7081 | tggtggtgct ctccagagtg tggaaccaag atagcactcg aacgacgaag taaagaacgc |
| 7141 | gaaaaagcgg aaaaagcagc agagaagaaa cgacgacgag aggagcagaa acagaaagat |
| 7201 | aaacttaaga ttcgaaaact cgccttaaag ccccgcagtt actggattaa acaagcccaa |
| 7261 | caagccgtaa acgccttcat cagagaaaga gaccgcgact taccatgtat ctcgtgcgga |
| 7321 | acgctcacgt ctgctcagtg ggatgccgga cattaccgga caactgctgc ggcacctcaa |
| 7381 | ctccgattta atgaacgcaa tattcacaag caatgcgtgg tgtgcaacca gcacaaaagc |
| 7441 | ggaaatctcg ttccgtatcg cgtcgaactg attagccgca tcgggcagga agcagtagac |
| 7501 | gaaatcgaat caaaccataa ccgccatcgc tggactatcg aagagtgcaa ggcgatcaag |
| 7561 | gcagagtacc aacagaaact caaagacctg cgaaatagca gaagtgaggc cgcatgacgt |
| 7621 | tctcagtaaa aaccattcca gacatgctcg ttgaagcata cggaaatcag acagaagtag |
| 7681 | cacgcagact gaaatgtagt cgcggtacgg tcagaaaata cgttgatgat aaagacggga |
| 7741 | aaatgcacgc catcgtcaac gacgttctca tggttcatcg cggatggagt gaaagagatg |
| 7801 | cgctattacg aaaaaattga tggcagcaaa taccgaaata tttgggtagt tggcgatctg |
| 7861 | cacggatgct acacgaacct gatgaacaaa ctggatacga ttggattcga caacaaaaaa |
| 7921 | gacctgctta tctcggtggg cgatttggtt gatcgtggtg cagagaacgt tgaatgcctg |
| 7981 | gaattaatca cattcccctg gttcagagct gtacgtggaa accatgagca aatgatgatt |
| 8041 | gatggcttat cagagcgtgg aaacgttaat cactggctgc ttaatggcgg tggctggttc |
| 8101 | tttaatctcg attacgacaa agaaattctg gctaaagctc ttgcccataa agcagatgaa |
| 8161 | cttccgttaa tcatcgaact ggtgagcaaa gataaaaaat atgttatctg ccacgccgat |
| 8221 | tatccctttg acgaatacga gtttggaaag ccagttgatc atcagcaggt aatctggaac |
| 8281 | cgcgaacgaa tcagcaactc acaaaacggg atcgtgaaag aaatcaaagg cgcggacacg |
| 8341 | ttcatctttg gtcatacgcc agcagtgaaa ccactcaagt ttgccaacca aatgtatatc |
| 8401 | gataccggcg cagtgttctg cggaaaccta acattgattc aggtacaggg agaaggcgca |
| 8461 | tgagactcga aagcgtagct aaatttcatt cgccaaaaag cccgatgatg agcgactcac |
| 8521 | cacgggccac ggcttctgac tctctttccg gtactgatgt gatggctgct atggggatgg |
| 8581 | cgcaatcaca agccggattc ggtatggctg cattctgcgg taagcacgaa ctcagccaga |
| 8641 | acgacaaaca aaaggctatc aactatctga tgcaatttgc acacaaggta tcggggaaat |
| 8701 | accgtggtgt ggcaaagctt gaaggaaata ctaaggcaaa ggtactgcaa gtgctcgcaa |
| 8761 | cattcgctta tgcggattat tgccgtagtg ccgcgacgcc gggggcaaga tgcagagatt |
| 8821 | gccatggtac aggccgtgcg gttgatattg ccaaaacaga gctgtggggg agagttgtcg |
| 8881 | agaaagagtg cggaagatgc aaaggcgtcg gctattcaag gatgccagca agcgcagcat |
| 8941 | atcgcgctgt gacgatgcta atcccaaacc ttacccaacc cacctggtca cgcactgtta |
| 9001 | agccgctgta tgacgctctg gtggtgcaat gccacaaaga agagtcaatc gcagacaaca |
| 9061 | ttttgaatgc ggtcacacgt tagcagcatg attgccacgg atggcaacat attaacggca |
| 9121 | tgatattgac ttattgaata aaattgggta aatttgactc aacgatgggt taattcgctc |
| 9181 | gttgtggtag tgagatgaaa agaggcggcg cttactaccg attccgccta gttggtcact |
| 9241 | tcgacgtatc gtctggaact ccaaccatcg caggcagaga ggtctgcaaa atgcaatccc |
| 9301 | gaaacagttc gcaggtaata gttagagcct gcataacggt ttcgggattt tttatatctg |
| 9361 | cacaacaggt aagagcattg agtcgataat cgtgaagagt cggcgagcct ggttagccag |
| 9421 | tgctctttcc gttgtgctga attaagcgaa taccggaagc agaaccggat caccaaatgc |
| 9481 | gtacaggcgt catcgccgcc cagcaacagc acaacccaaa ctgagccgta gccactgtct |
| 9541 | gtcctgaatt cattagtaat agttacgctg cggcctttta cacatgacct tcgtgaaagc |
| 9601 | gggtggcagg aggtcgcgct aacaacctcc tgccgttttg cccgtgcata tcggtcacga |
| 9661 | acaaatctga ttactaaaca cagtagcctg gatttgttct atcagtaatc gaccttattc |
| 9721 | ctaattaaat agagcaaatc cccttattgg gggtaagaca tgaagatgcc agaaaaacat |
| 9781 | gacctgttgg ccgccattct cgcggcaaag gaacaaggca tcggggcaat ccttgcgttt |
| 9841 | gcaatggcgt accttcgcgg cagatataat ggcggtgcgt ttacaaaaac agtaatcgac |
| 9901 | gcaacgatgt gcgccattat cgcctggttc attcgtgacc ttctcgactt cgccggacta |
| 9961 | agtagcaatc tcgcttatat aacgagcgtg tttatcggct acatcggtac tgactcgatt |
| 10021 | ggttcgctta tcaaacgctt cgctgctaaa aaagccggag tagaagatgg tagaaatcaa |
| 10081 | taatcaacgt aaggcgttcc tcgatatgct ggcgtggtcg gagggaactg ataacggacg |
| 10141 | tcagaaaacc agaaatcatg gttatgacgt cattgtaggc ggagagctat ttactgatta |
| 10201 | ctccgatcac cctcgcaaac ttgtcacgct aaacccaaaa ctcaaatcaa caggcgctta |
| 10261 | agactggccg tcgttttaca acacagaaag agtttgtaga aacgcaaaaa ggccatccgt |
| 10321 | caggggcctt ctgcttagtt tgatgcctgg cagttcccta ctctcgcctt ccgcttcctc |
| 10381 | gctcactgac tcgctgcgct cggtcgttcg gctgcggcga gcggtatcag ctcactcaaa |
| 10441 | ggcggtaata cggttatcca cagaatcagg ggataacgca ggaaagaaca tgtgagcaaa |
| 10501 | aggccagcaa aaggccagga accgtaaaaa ggccgcgttg ctggcgtttt tccataggct |
| 10561 | ccgcccccct gacgagcatc acaaaaatcg acgctcaagt cagaggtggc gaaacccgac |
| 10621 | aggactataa agataccagg cgtttccccc tggaagctcc ctcgtgcgct ctcctgttcc |
| 10681 | gaccctgccg cttaccggat acctgtccgc ctttctccct tcgggaagcg tggcgctttc |
| 10741 | tcatagctca cgctgtaggt atctcagttc ggtgtaggtc gttcgctcca agctgggctg |
| 10801 | tgtgcacgaa ccccccgttc agcccgaccg ctgcgcctta tccggtaact atcgtcttga |
| 10861 | gtccaacccg gtaagacacg acttatcgcc actggcagca gccactggta acaggattag |
| 10921 | cagagcgagg tatgtaggcg gtgctacaga gttcttgaag tggtgggcta actacggcta |
| 10981 | cactagaaga acagtatttg gtatctgcgc tctgctgaag ccagttacct tcggaaaaag |
| 11041 | agttggtagc tcttgatccg gcaaacaaac caccgctggt agcggtggtt tttttgtttg |
| 11101 | caagcagcag attacgcgca gaaaaaaagg atctcaagaa gatcctttga tcttttctac |
| 11161 | ggggtctgac gctcagtgga acgacgcgcg cgtaactcac gttaagggat tttggtcatg |
| 11221 | agcttgcgcc gtcccgtcaa gtcagcgtaa tgctctgctt ttagaaaaac tcatcgagca |
| 11281 | tcaaatgaaa ctgcaattta ttcatatcag gattatcaat accatatttt tgaaaaagcc |
| 11341 | gtttctgtaa tgaaggagaa aactcaccga ggcagttcca taggatggca agatcctggt |
| 11401 | atcggtctgc gattccgact cgtccaacat caatacaacc tattaatttc ccctcgtcaa |
| 11461 | aaataaggtt atcaagtgag aaatcaccat gagtgacgac tgaatccggt gagaatggca |
| 11521 | aaagtttatg catttctttc cagacttgtt caacaggcca gccattacgc tcgtcatcaa |
| 11581 | aatcactcgc atcaaccaaa ccgttattca ttcgtgattg cgcctgagcg aggcgaaata |
| 11641 | cgcgatcgct gttaaaagga caattacaaa caggaatcga gtgcaaccgg cgcaggaaca |
| 11701 | ctgccagcgc atcaacaata ttttcacctg aatcaggata ttcttctaat acctggaacg |
| 11761 | ctgtttttcc ggggatcgca gtggtgagta accatgcatc atcaggagta cggataaaat |
| 11821 | gcttgatggt cggaagtggc ataaattccg tcagccagtt tagtctgacc atctcatctg |
| 11881 | taacatcatt ggcaacgcta cctttgccat gtttcagaaa caactctggc gcatcgggct |
| 11941 | tcccatacaa gcgatagatt gtcgcacctg attgcccgac attatcgcga gcccatttat |
| 12001 | acccatataa atcagcatcc atgttggaat ttaatcgcgg cctcgacgtt tcccgttgaa |
| 12061 | tatggctcat attcttcctt tttcaatatt attgaagcat ttatcagggt tattgtctca |
| 12121 | tgagcggata catatttgaa tgtatttaga aaaataaaca aataggggtc agtgttacaa |
| 12181 | ccaattaacc aattctgaac attatcgcga gcccatttat acctgaatat ggctcataac |
| 12241 | accccttgtt tgcctggcgg cagtagcgcg gtggtcccac ctgaccccat gccgaactca |
| 12301 | gaagtgaaac gccgtagcgc cgatggtagt gtggggactc cccatgcgag agtagggaac |
| 12361 | tgccaggcat caaataaaac gaaaggctca gtcgaaagac tgggcctttc gcccgggcta |
| 12421 | attagggggt gtcgccctta ttcgactcta tagtgaagtt cctattctct agaaagtata |
| 12481 | ggaacttctg aagtggggtc gacttaatta agg |

These rAAV particles are tested for efficacy in cell culture and then administered to an animal model of an ABCA4-associated ocular disorder.

In the cell, for example, the 5′ RTM molecule that is designed to interact with a selected target pre-mRNA, e.g., human ABCA4. The RTM comprises a target binding domain, which is a sequence complementary to a portion of Intron 22 of ABCA4, a splicing domain, and a coding domain, with its sequence encoding wildtype Exon 1-22 of ABCA4. Upon delivery to the ocular cell in a recombinant AAV, the target binding domain, which is a sequence complementary to a portion of Intron 22 of ABCA4, binds to Intron 22 of the targeted defective/mutated gene, and the action of the spliceosome operates to replace the target coding wildtype Exon 1-22 of the 5′RTM for the subject's Exon 1-22, which contains defects resulting in disease. The RIM in vivo reprograms the subject's pre-mRNA in the cell, so that the cell now produces ABCA4 without the defects previously in the mutated gene. The same operation occurs with the delivery of the 3′ RTM via the rAAV and the ocular cells now have the ability to produce the normal wildtype or corrected gene.

### Example 3: Methods of Evaluating RTM Efficacy

ABCA4 is exclusively expressed in photoreceptors of the retina, and these cells are particularly challenging to culture ex vivo. On method of modeling model molecular correction of ABCA4 involves delivering a mixture of rAAV particles containing the 3′RTM and 5′RTM of Example 1 in normal cell culture of photoreceptors. The cells are permitted to grow in culture for a time sufficient to permit the RTM transgenes delivered by the rAAV to perform the trans-splicing function in the cells. Thereafter the cells will be analyzed by conventional methods for the presence of wildtype (or corrected) ABCA4.

Another method of modeling disease to determine the effect of the rAAV delivery of the RTMs is in personalized models using induced pluripotent stem (iPSC) cells obtained from patients diagnosed with Stargardt's in the clinic.

In still another method to facilitate ABCA4 RTM evaluation, an ABCA4 Intron 26 mini-gene is designed for analysis of trans-splicing. The mini-gene construct is created from a healthy donor genomic DNA pool and modified via polymerase chain reaction (PCR) to include a 5′ c-Myc tag and a 3′ 3xFLAG tag. Additionally, a 3′ IRES followed by a Puromycin resistance gene allows for positive selection of cells containing the mini-gene. One such recombinant construct comprises a Myc protein tag, Exon26-Intron 26-Exon 27 of human ABCA4, a 3xFLAG protein tag, an IRES, and an antibiotic resistance gene, under the control of regulatory sequences which can express the product of said gene in selected mammalian host cell.

This construct is cloned into the pK1 retroviral vector, and recombinant virus is generated by triple transfection. The recombinant virus carrying the minigene is transduced into HEK293T cells. With puromycin selection, a stably selected 293T-ABCA4-Int26 mg cell line is created. This mini-gene design allows bidirectional reporting for both 5′ and 3′ trans-splicing. This cell line is used for preliminary analysis of the ABCA4 RNA trans-splicing molecules.

In a similar manner, a mini-gene for intron 22 is provided to facilitate evaluation of 5′ RTMs for ABCA4.

### Example 4—RTM Assembly for LCA10

In another embodiment a 5′ RTM is designed with a binding domain targeting intron 26 of CLP290 comprises:
3′ RTM promoter
Binding domain sequence: 70-200 nucleotides complementary to target intron 26;

3′RTM spacer sequence:
SEQ ID NO: 13
AGAGCTCGTTGCGATATTAT
3′RTM BP:
TACTAAC
3′RTM PPT:
SEQ ID NO: 14
TGGTACCTCTTCTTTTTTTTCTG
3′ Splice Site:
CAGGT

Coding domain of CP290 cDNA encoding exons 1 through the exon 26; 3′ RTM PolyA signal sequence.

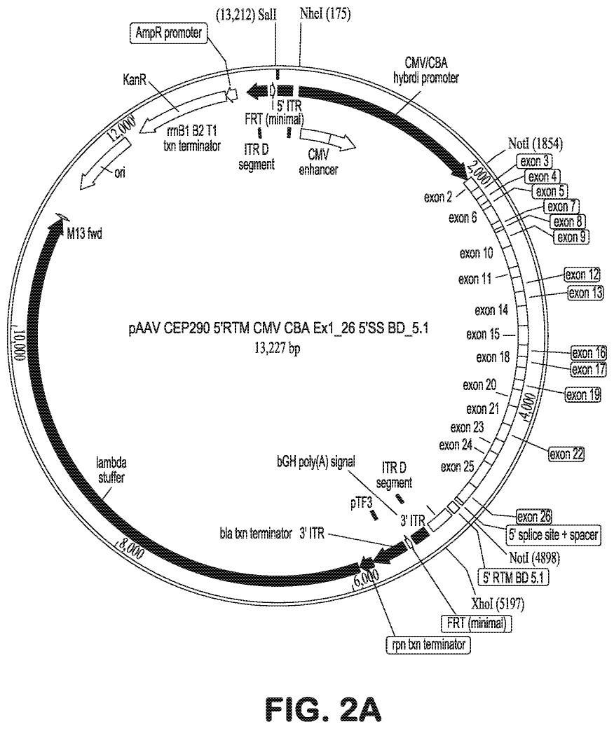

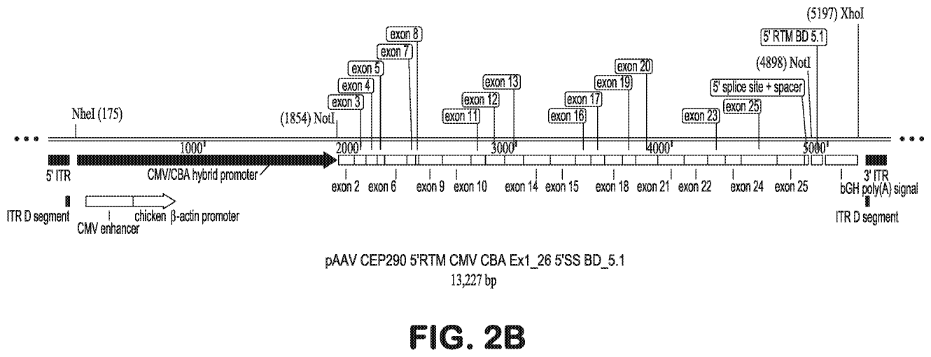

Each RTM is introduced into a proviral plasmid p618 as referenced above, following the teachings of WO2012/158757. The proviral plasmids are cultured in the host cells which express the cap and/or rep proteins. In the host cells, each minigene consisting of the RTM with flanking AAV ITRs is rescued and packaged into the capsid protein or envelope protein to form an infectious viral particle. Thus two types of recombinant AAV infectious particle are produced and purified from culture: one carrying the 3′RTM and the other carrying the 5′RTM. See, e.g., FIGS. 2A and 2B and TABLE 5, which is the sequence of the RTM of FIG. 2A in GenBank format which delineates features of the sequence.

TABLE 5
pAAV CEP290 5′RTM CMV CBA Ex1 26 5′SS BD_5.1
synthetic DNA construct - recombinant plasmid
13227 bp ds-DNA circular
REFERENCE 1 (bases 1 to 13227) SEQ ID NO: 2

| Features | Location/Qualifiers |
|---|---|
| source | 1 . . . 13227 |
| | /organism=“recombinant plasmid” |
| | /mol_type=“other DNA” |
| repeat_region | 1 . . . 130 |
| | /note=“5′ ITR” |
| misc_feature | 113 . . . 130 |
| | /note=“ITR D segment” |
| promoter | 191 . . . 1852 |
| | /note=“CMV/CBA hybrid promoter” |
| enhancer | 241 . . . 544 |
| | /note=“CMV enhancer” |
| | /note=“human cytomegalovirus immediate early enhancer” |
| promoter | 546 . . . 823 |
| | /note=“chicken beta-actin promoter” |
| exon | 1861 . . . 1962 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 2” |
| | /inference=“alignment:same species:1.39.8” |
| exon | 1963 . . . 2040 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 3” |
| | /inference=“alignment:same species:1.39.8” |
| exon | 2041.2110 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 4” |
| | /inference=“alignment:same species:1.39.8” |
| exon | 2111.2157 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 5” |
| | /inference=“alignment:same species:1.39.8” |
| exon | 2158 . . . 2301 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 6” |
| | /inference=“alignment:same species:1.39.8” |
| exon | 2302 . . . 2355 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 7” |
| | /inference=“alignment:same species:1.39.8” |
| exon | 2356 . . . 2376 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 8” |
| | /inference=“alignment:same species:1.39.8” |
| exon | 2377 . . . 2529 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 9” |
| | /inference=“alignment:same species:1.39.8” |
| exon | 2530 . . . 2712 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 10” |
| | /inference=“alignment:same species:1.39.8” |
| exon | 2713 . . . 2802 |
| | /gene=“CEP290” |
| | /gene synonym=“3H11Ag; BBS14; CT87; JBTS5; LCA10; MKS4; |
| | NPHP6; POC3; rd16; SLSN6” |
| | /note=“exon 11” |
| | /inference=“alignment:same species:1.39.8” |
| exon | 2803 . . . 2925 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 12” |
| | /inference=“alignment:same species:1.39.8” |
| exon | 2926 . . . 3049 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 13” |
| | /inference=“alignment:same species:1.39.8” |
| exon | 3050 . . . 3219 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 14” |
| | /inference=“alignment:same species:1.39.8” |
| exon | 3220 . . . 3382 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 15” |
| | /inference=“alignment:same species:1.39.8” |
| exon | 3383.3483 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 16” |
| | /inference=“alignment:same species:1.39.8” |
| exon | 3484 . . . 3571 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note-”exon 17” |
| | /inference=“alignment:same species:1.39.8” |
| exon | 3572 . . . 3684 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 18” |
| | /inference=“alignment:same species:1.39.8” |
| exon | 3685 . . . 3769 |
| | /gene=“CEP290” |
| | /gene_synonym=“3H11Ag; BBS14; CT87; JBTS5; LCA10; MKS4; |
| | NPHP6; POC3; rd16; SLSN6” |
| | /note=“exon 19” |
| | /inference=“alignment:same species:1.39.8” |
| exon | 3770 . . . 3912 |
| | /gene=“CEP290” |
| | /gene_synonym=“3H11Ag; BBS14; CT87; JBTS5; |
| | LCA10; MKS4; |
| | NPHP6; POC3; rd16; SLSN6” |
| | /note=“exon 20” |
| | /inference=“alignment:same species:1.39.8” |
| exon | 3913 . . . 4077 |
| | /gene=“CEP290” |
| | /gene_synonym=“3H11Ag; BBS14; CT87; JBTS5; |
| | LCA10; MKS4; |
| | NPHP6; POC3; rd16; SLSN6” |
| | /note=“exon 21” |
| | /inference=“alignment:same species:1.39.8” |
| exon | 4078 . . . 4227 |
| | /gene=“CEP290” |
| | /gene_synonym=“3H11Ag; BBS14; CT87; JBTS5; |
| | LCA10; MKS4; |
| | NPHP6; POC3; rd16; SLSN6” |
| | /note=“exon 22” |
| | /inference=“alignment:same species:1.39.8” |
| exon | 4228 . . . 4343 |
| | /gene=“CEP290” |
| | /gene_synonym=“3H11Ag; BBS14; CT87; JBTS5; |
| | LCA10; MKS4; |
| | NPHP6; POC3; rd16; SLSN6” |
| | /note=“exon 23” |
| | /inference=“alignment:same species:1.39.8” |
| exon | 4344 . . . 4446 |
| | /gene=“CEP290” |
| | /gene_synonym=“3H11Ag; BBS14; CT87; JBTS5; |
| | LCA10; MKS4; |
| | NPHP6; POC3; rd16; SLSN6” |
| | /note=“exon 24” |
| | /inference=“alignment:same species:1.39.8” |
| exon | 4447 . . . 4677 |
| | /gene=“CEP290” |
| | /gene_synonym=“3H11Ag; BBS14; CT87; JBTS5; |
| | LCA10; MKS4; |
| | NPHP6; POC3; rd16; SLSN6” |
| | /note=“exon 25” |
| | /inference=“alignment:same species:1.39.8” |
| exon | 4678 . . . 4851 |
| | /gene=“CEP290” |
| | /gene_synonym=“3H11Ag; BBS14; CT87; JBTS5; |
| | LCA10; MKS4; |
| | NPHP6; POC3; rd16; SLSN6” |
| | /note=“exon 26” |
| | /inference=“alignment:same species:1.39.8” |
| misc_feature | 4853 . . . 4876 |
| | /note=“5′ splice site + spacer” |
| misc_feature | 4899.4967 |
| | /note=“5′ RTM BD 5.1” |
| polyA_signal | 4989 . . . 5196 |
| | /note=“bGH poly(A) signal” |
| | /note=“bovine growth hormone polyadenylation signal” |
| repeat_region | 5246 . . . 5375 |
| | /note=“3′ ITR” |
| misc_feature | 5246 . . . 5263 |
| | /note=“ITR D segment” |
| protein_bind | complement(5403 . . . 5436) |
| | /bound_moiety=“FLP recombinase from the Saccharomyces cerevisiae |
| | 2u plasmid” |
| | /note=“FRT (minimal)” |
| | /note=“supports FLP-mediated excision but not integration |
| | (Turan and Bode, 2011)” |
| misc_feature | 5469 . . . 5769 |
| | /product=“bla txn terminator” |
| | /note=“bla txn terminator” |
| misc_feature | 5560 . . . 5585 |
| | /product=“pTF3” |
| | /note=“pTF3” |
| misc_feature | 5776 . . . 5889 |
| | /product=“rpn txn terminator” |
| | /note=“rpn txn terminator” |
| misc_feature | 5905 . . . 10971 |
| | /note=“lambda stuffer” |
| primer_bind | complement(10977.10993) |
| | /note=“M13 fwd” |
| | /note=“common sequencing primer, one of multiple similar |
| | variants” |
| rep_origin | complement(11263 . . . 11851) |
| | /direction=LEFT |
| | /note=“ori” |
| | /note=“high-copy-number ColEl/pMB1/pBR322/pUC |
| | origin of |
| | replication” |
| CDS SEQ | complement(11975 . . . 12784) |
| ID NO: 4 | /codon start=1 |
| | /gene=“aph(3′)-Ia” |
| | /product=“aminoglycoside phosphotransferase” |
| | /note=“KanR” |
| | /note=“confers resistance to kanamycin in bacteria or G418 |
| | (Geneticin(R)) in eukaryotes” |
| | /translation=“MSHIQRETSRPRLNSNMDADLYGYKW |
| | ARDNVGQSGATIYRLYGKPDAPELFLKHGKGSVANDVTD |
| | EMVRLNWLTEFMPLPTIKHFIRTPDDAWLLTTAIPGKT |
| | AFQVLEEYPDSGENIVDALAVFLRRLHSIPVCNCPFNS |
| | DRVFRLAQAQSRMNNGLVDASDFDDERNGWPVEQVWK |
| | EMHKLLPFSPDSVVTHGDFSLDNLIFDEGKLIGCIDV |
| | GRVGIADRYQDLAILWNCLGEFSPSLQKRLFQKYGIDN |
| | PDMNKLQFHLMLDEFF” |
| promoter | complement(12785 . . . 12876) |
| | /gene=“bla” |
| | /note=“AmpR promoter” |
| misc_feature | complement(12963 . . . 13137) |
| | /product=“rmnB1 B2 T1 txn terminator” |
| | /note=“rmnB1 B2 T1 txn terminator” |
| misc_feature | 13038 . . . 13054 |
| | /product=“pTR” |
| | /note=“pTR” |
| protein_bind | 13169 . . . 13202 |
| | /bound_moiety=“FLP recombinase from the |
| | Saccharomyces cerevisiae 2u plasmid” |
| | /note=“FRT (minimal)” |
| | /note=“supports FLP-mediated excision but not integration |
| | (Turan and Bode, 2011)” |

```
ORIGIN SEQ ID NO: 2
        1 ctgcgcgctc gctcgctcac tgaggccgcc cgggcaaagc ccgggcgtcg ggcgaccttt
      61 ggtcgcccgg cctcagtgag cgagcgagcg cgcagagagg gagtggccaa ctccatcact
    121 aggggttcct tgtagttaat gattaacccg ccatgctact tatctacgta gcaagctagc
    181 tagttattaa tagtaatcaa ttacggggtc attagttcat agcccatata tggagttccg
    241 cgttacataa cttacggtaa atggcccgcc tggctgaccg cccaacgacc cccgcccatt
    301 gacgtcaata atgacgtatg ttcccatagt aacgccaata gggactttcc attgacgtca
    361 atgggtggag tatttacggt aaactgccca cttggcagta catcaagtgt atcatatgcc
    421 aagtacgccc cctattgacg tcaatgacgg taaatggccc gcctggcatt atgcccagta
    481 catgacctta tgggactttc ctacttggca gtacatctac gtattagtca tcgctattaa
    541 catggtcgag gtgagcccca cgttctgctt cactctcccc atctcccccc cctccccacc
    601 cccaattttg tatttattta ttttttaatt attttgtgca gcgatggggg cggggggggg
    661 gggggggcgc gcgccaggcg gggcggggcg gggcgagggg cggggcgggg cgaggcggag
    721 aggtgcggcg gcagccaatc agagcggcgc gctccgaaag tttcctttta tggcgaggcg
    781 gcggcggcgg cggccctata aaaagcgaag cgcgcggcgg gcggggagtc gctgcgacgc
    841 tgccttcgcc ccgtgccccg ctccgccgcc gcctcgcgcc gcccgccccg gctctgactg
    901 accgcgttac tcccacaggt gagcgggcgg gacggccctt ctcctccggg ctgtaattag
    961 cgcttggttt aatgacggct tgtttctttt ctgtggctgc gtgaaagcct tgaggggctc
  1021 cgggagggcc ctttgtgcgg ggggagcggc tcggggggtg cgtgcgtgtg tgtgtgcgtg
  1081 gggagcgccg cgtgcggctc cgcgctgccc ggcggctgtg agcgctgcgg gcgcggcgcg
  1141 gggctttgtg cgctccgcag tgtgcgcgag gggagcgcgg ccgggggcgg tgccccgcgg
  1201 tgcggggggg gctgcgaggg gaacaaaggc tgcgtgcggg gtgtgtgcgt gggggggtga
  1261 gcagggggtg tgggcgcgtc ggtcgggctg caaccccccc tgcacccccc tccccgagtt
  1321 gctgagcacg gcccggcttc gggtgcgggg ctccgtacgg ggcgtggcgc ggggctcgcc
  1381 gtgccgggcg gggggtggcg gcaggtgggg gtgccgggcg gggcggggcc gcctcgggcc
  1441 ggggagggct cgggggaggg gcgcggcggc ccccggagcg ccggcggctg tcgaggcgcg
  1501 gcgagccgca gccattgcct tttatggtaa tcgtgcgaga gggcgcaggg acttcctttg
  1561 tcccaaatct gtgcggagcc gaaatctggg aggcgccgcc gcaccccctc tagcgggcgc
  1621 ggggcgaagc ggtgcggcgc cggcaggaag gaaatgggcg gggagggcct tcgtgcgtcg
  1681 ccgcgccgcc gtccccttct ccctctccag cctcggggct gtccgcgggg ggacggctgc
  1741 cttcgggggg gacggggcag ggcggggttc ggcttctggc gtgtgaccgg cggctctaga
  1801 caattgtact aaccttcttc tctttcctct cctgacaggt tggtgtacac tagcgggcgc
  1861 atgccaccta atataaactg gaaagaaata atgaaagttg acccagatga cctgccccgt
  1921 caagaagaac tggcagataa tttattgatt tccttatcca aggtggaagt aaatgagcta
  1981 aaaagtgaaa agcaagaaaa tgtgatacac cttttcagaa ttactcagtc actaatgaag
  2041 atgaaagctc aagaagtgga gctggctttg gaagaagtag aaaaagctgg agaagaacaa
  2101 gcaaaatttg aaaatcaatt aaaaactaaa gtaatgaaac tggaaaatga actggagatg
  2161 gctcagcagt ctgcaggtgg acgagatact cggtttttac gtaatgaaat ttgccaactt
  2221 gaaaaacaat tagaacaaaa agatagagaa ttggaggaca tggaaaagga gttggagaaa
  2281 gagaagaaag ttaatgagca attggctctt cgaaatgagg aggcagaaaa tgaaaacagc
  2341 aaattaagaa gagagaacaa acgtctaaag aaaaagaatg aacaactttg tcaggatatt
  2401 attgactacc agaaacaaat agattcacag aaagaaacac ttttatcaag aagaggggaa
  2461 gacagtgact accgatcaca gttgtctaaa aaaaactatg agcttatcca atatcttgat
  2521 gaaattcaga ctttaacaga agctaatgag aaaattgaag ttcagaatca agaaatgaga
  2581 aaaaatttag aagagtctgt acaggaaatg gagaagatga ctgatgaata taatagaatg
  2641 aaagctattg tgcatcagac agataatgta atagatcagt taaaaaaaga aaacgatcat
  2701 tatcaacttc aagtgcagga gcttacagat cttctgaaat caaaaaatga agaagatgat
  2761 ccaattatgg tagctgtcaa tgcaaaagta gaagaatgga agctaatttt gtcttctaaa
  2821 gatgatgaaa ttattgagta tcagcaaatg ttacataacc taagggagaa acttaagaat
  2881 gctcagcttg atgctgataa aagtaatgtt atggctctac agcagggtat acaggaacga
  2941 gacagtcaaa ttaagatgct caccgaacaa gtagaacaat atacaaaaga aatggaaaag
  3001 aatacttgta ttattgaaga tttgaaaaat gagctccaaa gaaacaaagg tgcttcaacc
  3061 ctttctcaac agactcatat gaaaattcag tcaacgttag acattttaaa agagaaaact
  3121 aaagaggctg agagaacagc tgaactggct gaggctgatg ctagggaaaa ggataaagaa
  3181 ttagttgagg ctctgaagag gttaaaagat tatgaatcgg gagtatatgg tttagaagat
  3241 gctgtcgttg aaataaagaa ttgtaaaaac caaattaaaa taagagatcg agagattgaa
  3301 atattaacaa aggaaatcaa taaacttgaa ttgaagatca gtgatttcct tgatgaaaat
  3361 gaggcactta gagagcgtgt gggccttgaa ccaaagacaa tgattgattt aactgaattt
  3421 agaaatagca aacacttaaa acagcagcag tacagagctg aaaaccagat tcttttgaaa
  3481 gagattgaaa gtctagagga agaacgactt gatctgaaaa aaaaaattcg tcaaatggct
  3541 caagaaagag gaaaaagaag tgcaacttca ggattaacca ctgaggacct gaacctaact
  3601 gaaaacattt ctcaaggaga tagaataagt gaaagaaaat tggatttatt gagcctcaaa
  3661 aatatgagtg aagcacaatc aaagaatgaa tttctttcaa gagaactaat tgaaaaagaa
  3721 agagatttag aaaggagtag gacagtgata gccaaatttc agaataaatt aaaagaatta
  3781 gttgaagaaa ataagcaact tgaagaaggt atgaaagaaa tattgcaagc aattaaggaa
  3841 atgcagaaag atcctgatgt taaaggagga gaaacatctc taattatccc tagccttgaa
  3901 agactagtta atgctataga atcaaagaat gcagaaggaa tctttgatgc gagtctgcat
  3961 ttgaaagccc aagttgatca gcttaccgga agaaatgaag aattaagaca ggagctcagg
  4021 gaatctcgga aagaggctat aaattattca cagcagttgg caaaagctaa tttaaagata
  4081 gaccatcttg aaaaagaaac tagtctttta cgacaatcag aaggatcgaa tgttgttttt
  4141 aaaggaattg acttacctga tgggatagca ccatctagtg ccagtatcat taattctcag
  4201 aatgaatatt taatacattt gttacaggaa ctagaaaata aagaaaaaaa gttaaagaat
  4261 ttagaagatt ctcttgaaga ttacaacaga aaatttgctg taattcgtca tcaacaaagt
  4321 ttgttgtata aagaatacct aagtgaaaag gagacctgga aaacagaatc taaaacaata
  4381 aaagaggaaa agagaaaact tgaggatcaa gtccaacaag atgctataaa agtaaaagaa
  4441 tataataatt tgctcaatgc tcttcagatg gattcggatg aaatgaaaaa aatacttgca
  4501 gaaaatagta ggaaaattac tgttttgcaa gtgaatgaaa aatcacttat aaggcaatat
  4561 acaaccttag tagaattgga gcgacaactt agaaaagaaa atgagaagca aaagaatgaa
  4621 ttgttgtcaa tggaggctga agtttgtgaa aaaattgggt gtttgcaaag atttaaggaa
  4681 atggccattt tcaagattgc agctctccaa aaagttgtag ataatagtgt ttctttgtct
  4741 gaactagaac tggctaataa acagtacaat gaactgactg ctaagtacag ggacatcttg
  4801 caaaaagata atatgcttgt tcaaagaaca agtaacttgg aacacctgga ggtaagagag
  4861 ctcgttgcga tattattaca gatatccagc acagtggcgg ccgctgtaat cccagcactt
  4921 taggaggccg aggcgggtgg atcacgagtt caggagatcg acaccgcggt tcgaaagatc
  4981 tgcctcgact gtgccttcta gttgccagcc atctgttgtt tgcccctccc ccgtgccttc
  5041 cttgaccctg gaaggtgcca ctcccactgt cctttcctaa taaaatgagg aaattgcatc
  5101 gcattgtctg agtaggtgtc attctattct ggggggtggg gtggggcagg acagcaaggg
  5161 ggaggattgg gaagacaata gcaggcatgc tggggactcg agttctacgt agataagtag
  5221 catggcgggt taatcattaa ctacaaggaa cccctagtga tggagttggc cactccctct
  5281 ctgcgcgctc gctcgctcac tgaggccggg cgaccaaagg tcgcccgacg cccgggcttt
  5341 gcccgggcgg cctcagtgag cgagcgagcg cgcagcctta attaacctaa ggaaaatgaa
  5401 gtgaagttcc tatactttct agagaatagg aacttctata gtgagtcgaa taagggcgac
  5461 acaaaattta ttctaaatgc ataataaata ctgataacat cttatagttt gtattatatt
  5521 ttgtattatc gttgacatgt ataattttga tatcaaaaac tgattttccc tttattattt
  5581 tcgagattta ttttcttaat tctctttaac aaactagaaa tattgtatat acaaaaaatc
  5641 ataaataata gatgaatagt ttaattatag gtgttcatca atcgaaaaag caacgtatct
  5701 tatttaaagt gcgttgcttt tttctcattt ataaggttaa ataattctca tatatcaagc
  5761 aaagtgacag gcgcccttaa atattctgac aaatgctctt tccctaaact ccccccataa
  5821 aaaaacccgc cgaagcgggt ttttacgtta tttgcggatt aacgattact cgttatcaga
  5881 accgcccagg gggcccgagc ttaacctttt tatttggggg agagggaagt catgaaaaaa
  5941 ctaacctttg aaattcgatc tccagcacat cagcaaaacg ctattcacgc agtacagcaa
  6001 atccttccag acccaaccaa accaatcgta gtaaccattc aggaacgcaa ccgcagctta
  6061 gaccaaaaca ggaagctatg ggcctgctta ggtgacgtct ctcgtcaggt tgaatggcat
  6121 ggtcgctggc tggatgcaga aagctggaag tgtgtgttta ccgcagcatt aaagcagcag
  6181 gatgttgttc ctaaccttgc cgggaatggc tttgtggtaa taggccagtc aaccagcagg
  6241 atgcgtgtag gcgaatttgc ggagctatta gagcttatac aggcattcgg tacagagcgt
  6301 ggcgttaagt ggtcagacga agcgagactg gctctggagt ggaaagcgag atggggagac
  6361 agggctgcat gataaatgtc gttagtttct ccggtggcag gacgtcagca tatttgctct
  6421 ggctaatgga gcaaaagcga cgggcaggta aagacgtgca ttacgttttc atggatacag
  6481 gttgtgaaca tccaatgaca tatcggtttg tcagggaagt tgtgaagttc tgggatatac
  6541 cgctcaccgt attgcaggtt gatatcaacc cggagcttgg acagccaaat ggttatacgg
  6601 tatgggaacc aaaggatatt cagacgcgaa tgcctgttct gaagccattt atcgatatgg
  6661 taaagaaata tggcactcca tacgtcggcg gcgcgttctg cactgacaga ttaaaactcg
  6721 ttcccttcac caaatactgt gatgaccatt tcgggcgagg gaattacacc acgtggattg
  6781 gcatcagagc tgatgaaccg aagcggctaa agccaaagcc tggaatcaga tatcttgctg
  6841 aactgtcaga ctttgagaag gaagatatcc tcgcatggtg gaagcaacaa ccattcgatt
  6901 tgcaaatacc ggaacatctc ggtaactgca tattctgcat taaaaaatca acgcaaaaaa
  6961 tcggacttgc ctgcaaagat gaggagggat tgcagcgtgt ttttaatgag gtcatcacgg
  7021 gatcccatgt gcgtgacgga catcgggaaa cgccaaagga gattatgtac cgaggaagaa
  7081 tgtcgctgga cggtatcgcg aaaatgtatt cagaaaatga ttatcaagcc ctgtatcagg
  7141 acatggtacg agctaaaaga ttcgataccg gctcttgttc tgagtcatgc gaaatatttg
  7201 gagggcagct tgatttcgac ttcgggaggg aagctgcatg atgcgatgtt atcggtgcgg
  7261 tgaatgcaaa gaagataacc gcttccgacc aaatcaacct tactggaatc gatggtgtct
  7321 ccggtgtgaa agaacaccaa caggggtgtt accactaccg caggaaaagg aggacgtgtg
  7381 gcgagacagc gacgaagtat caccgacata atctgcgaaa actgcaaata ccttccaacg
  7441 aaacgcacca gaaataaacc caagccaatc ccaaaagaat ctgacgtaaa aaccttcaac
  7501 tacacggctc acctgtggga tatccggtgg ctaagacgtc gtgcgaggaa aacaaggtga
  7561 ttgaccaaaa tcgaagttac gaacaagaaa gcgtcgagcg agctttaacg tgcgctaact
  7621 gcggtcagaa gctgcatgtg ctggaagttc acgtgtgtga gcactgctgc gcagaactga
  7681 tgagcgatcc gaatagctcg atgcacgagg aagaagatga tggctaaacc agcgcgaaga
  7741 cgatgtaaaa acgatgaatg ccgggaatgg tttcaccctg cattcgctaa tcagtggtgg
  7801 tgctctccag agtgtggaac caagatagca ctcgaacgac gaagtaaaga acgcgaaaaa
  7861 gcggaaaaag cagcagagaa gaaacgacga cgagaggagc agaaacagaa agataaactt
  7921 aagattcgaa aactcgcctt aaagccccgc agttactgga ttaaacaagc ccaacaagcc
  7981 gtaaacgcct tcatcagaga aagagaccgc gacttaccat gtatctcgtg cggaacgctc
  8041 acgtctgctc agtgggatgc cggacattac cggacaactg ctgcggcacc tcaactccga
  8101 tttaatgaac gcaatattca caagcaatgc gtggtgtgca accagcacaa aagcggaaat
  8161 ctcgttccgt atcgcgtcga actgattagc cgcatcgggc aggaagcagt agacgaaatc
  8221 gaatcaaacc ataaccgcca tcgctggact atcgaagagt gcaaggcgat caaggcagag
  8281 taccaacaga aactcaaaga cctgcgaaat agcagaagtg aggccgcatg acgttctcag
  8341 taaaaaccat tccagacatg ctcgttgaag catacggaaa tcagacagaa gtagcacgca
  8401 gactgaaatg tagtcgcggt acggtcagaa aatacgttga tgataaagac gggaaaatgc
  8461 acgccatcgt caacgacgtt ctcatggttc atcgcggatg gagtgaaaga gatgcgctat
  8521 tacgaaaaaa ttgatggcag caaataccga aatatttggg tagttggcga tctgcacgga
  8581 tgctacacga acctgatgaa caaactggat acgattggat tcgacaacaa aaaagacctg
  8641 cttatctcgg tgggcgattt ggttgatcgt ggtgcagaga acgttgaatg cctggaatta
  8701 atcacattcc cctggttcag agctgtacgt ggaaaccatg agcaaatgat gattgatggc
  8761 ttatcagagc gtggaaacgt taatcactgg ctgcttaatg gcggtggctg gttctttaat
  8821 ctcgattacg acaaagaaat tctggctaaa gctcttgccc ataaagcaga tgaacttccg
  8881 ttaatcatcg aactggtgag caaagataaa aaatatgtta tctgccacgc cgattatccc
  8941 tttgacgaat acgagtttgg aaagccagtt gatcatcagc aggtaatctg gaaccgcgaa
  9001 cgaatcagca actcacaaaa cgggatcgtg aaagaaatca aaggcgcgga cacgttcatc
  9061 tttggtcata cgccagcagt gaaaccactc aagtttgcca accaaatgta tatcgatacc
  9121 ggcgcagtgt tctgcggaaa cctaacattg attcaggtac agggagaagg cgcatgagac
  9181 tcgaaagcgt agctaaattt cattcgccaa aaagcccgat gatgagcgac tcaccacggg
  9241 ccacggcttc tgactctctt tccggtactg atgtgatggc tgctatgggg atggcgcaat
  9301 cacaagccgg attcggtatg gctgcattct gcggtaagca cgaactcagc cagaacgaca
  9361 aacaaaaggc tatcaactat ctgatgcaat ttgcacacaa ggtatcgggg aaataccgtg
  9421 gtgtggcaaa gcttgaagga aatactaagg caaaggtact gcaagtgctc gcaacattcg
  9481 cttatgcgga ttattgccgt agtgccgcga cgccgggggc aagatgcaga gattgccatg
  9541 gtacaggccg tgcggttgat attgccaaaa cagagctgtg ggggagagtt gtcgagaaag
  9601 agtgcggaag atgcaaaggc gtcggctatt caaggatgcc agcaagcgca gcatatcgcg
  9661 ctgtgacgat gctaatccca aaccttaccc aacccacctg gtcacgcact gttaagccgc
  9721 tgtatgacgc tctggtggtg caatgccaca aagaagagtc aatcgcagac aacattttga
  9781 atgcggtcac acgttagcag catgattgcc acggatggca acatattaac ggcatgatat
  9841 tgacttattg aataaaattg ggtaaatttg actcaacgat gggttaattc gctcgttgtg
  9901 gtagtgagat gaaaagaggc ggcgcttact accgattccg cctagttggt cacttcgacg
  9961 tatcgtctgg aactccaacc atcgcaggca gagaggtctg caaaatgcaa tcccgaaaca
10021 gttcgcaggt aatagttaga gcctgcataa cggtttcggg attttttata tctgcacaac
10081 aggtaagagc attgagtcga taatcgtgaa gagtcggcga gcctggttag ccagtgctct
10141 ttccgttgtg ctgaattaag cgaataccgg aagcagaacc ggatcaccaa atgcgtacag
10201 gcgtcatcgc cgcccagcaa cagcacaacc caaactgagc cgtagccact gtctgtcctg
10261 aattcattag taatagttac gctgcggcct tttacacatg accttcgtga aagcgggtgg
10321 caggaggtcg cgctaacaac ctcctgccgt tttgcccgtg catatcggtc acgaacaaat
10381 ctgattacta aacacagtag cctggatttg ttctatcagt aatcgacctt attcctaatt
10441 aaatagagca aatcccctta ttgggggtaa gacatgaaga tgccagaaaa acatgacctg
10501 ttggccgcca ttctcgcggc aaaggaacaa ggcatcgggg caatccttgc gtttgcaatg
10561 gcgtaccttc gcggcagata taatggcggt gcgtttacaa aaacagtaat cgacgcaacg
10621 atgtgcgcca ttatcgcctg gttcattcgt gaccttctcg acttcgccgg actaagtagc
10681 aatctcgctt atataacgag cgtgtttatc ggctacatcg gtactgactc gattggttcg
10741 cttatcaaac gcttcgctgc taaaaaagcc ggagtagaag atggtagaaa tcaataatca
10801 acgtaaggcg ttcctcgata tgctggcgtg gtcggaggga actgataacg gacgtcagaa
10861 aaccagaaat catggttatg acgtcattgt aggcggagag ctatttactg attactccga
10921 tcaccctcgc aaacttgtca cgctaaaccc aaaactcaaa tcaacaggcg cttaagactg
10981 gccgtcgttt tacaacacag aaagagtttg tagaaacgca aaaaggccat ccgtcagggg
11041 ccttctgctt agtttgatgc ctggcagttc cctactctcg ccttccgctt cctcgctcac
11101 tgactcgctg cgctcggtcg ttcggctgcg gcgagcggta tcagctcact caaaggcggt
11161 aatacggtta tccacagaat caggggataa cgcaggaaag aacatgtgag caaaaggcca
11221 gcaaaaggcc aggaaccgta aaaaggccgc gttgctggcg tttttccata ggctccgccc
11281 ccctgacgag catcacaaaa atcgacgctc aagtcagagg tggcgaaacc cgacaggact
11341 ataaagatac caggcgtttc cccctggaag ctccctcgtg cgctctcctg ttccgaccct
11401 gccgcttacc ggatacctgt ccgcctttct cccttcggga agcgtggcgc tttctcatag
11461 ctcacgctgt aggtatctca gttcggtgta ggtcgttcgc tccaagctgg gctgtgtgca
11521 cgaacccccc gttcagcccg accgctgcgc cttatccggt aactatcgtc ttgagtccaa
11581 cccggtaaga cacgacttat cgccactggc agcagccact ggtaacagga ttagcagagc
11641 gaggtatgta ggcggtgcta cagagttctt gaagtggtgg gctaactacg gctacactag
11701 aagaacagta tttggtatct gcgctctgct gaagccagtt accttcggaa aaagagttgg
11761 tagctcttga tccggcaaac aaaccaccgc tggtagcggt ggtttttttg tttgcaagca
11821 gcagattacg cgcagaaaaa aaggatctca agaagatcct ttgatctttt ctacggggtc
11881 tgacgctcag tggaacgacg cgcgcgtaac tcacgttaag ggattttggt catgagcttg
11941 cgccgtcccg tcaagtcagc gtaatgctct gcttttagaa aaactcatcg agcatcaaat
12001 gaaactgcaa tttattcata tcaggattat caataccata tttttgaaaa agccgtttct
12061 gtaatgaagg agaaaactca ccgaggcagt tccataggat ggcaagatcc tggtatcggt
12121 ctgcgattcc gactcgtcca acatcaatac aacctattaa tttcccctcg tcaaaaataa
12181 ggttatcaag tgagaaatca ccatgagtga cgactgaatc cggtgagaat ggcaaaagtt
12241 tatgcatttc tttccagact tgttcaacag gccagccatt acgctcgtca tcaaaatcac
12301 tcgcatcaac caaaccgtta ttcattcgtg attgcgcctg agcgaggcga aatacgcgat
12361 cgctgttaaa aggacaatta caaacaggaa tcgagtgcaa ccggcgcagg aacactgcca
12421 gcgcatcaac aatattttca cctgaatcag gatattcttc taatacctgg aacgctgttt
12481 ttccggggat cgcagtggtg agtaaccatg catcatcagg agtacggata aaatgcttga
12541 tggtcggaag tggcataaat tccgtcagcc agtttagtct gaccatctca tctgtaacat
12601 cattggcaac gctacctttg ccatgtttca gaaacaactc tggcgcatcg ggcttcccat
12661 acaagcgata gattgtcgca cctgattgcc cgacattatc gcgagcccat ttatacccat
12721 ataaatcagc atccatgttg gaatttaatc gcggcctcga cgtttcccgt tgaatatggc
12781 tcatattctt cctttttcaa tattattgaa gcatttatca gggttattgt ctcatgagcg
12841 gatacatatt tgaatgtatt tagaaaaata aacaaatagg ggtcagtgtt acaaccaatt
12901 aaccaattct gaacattatc gcgagcccat ttatacctga atatggctca taacacccct
12961 tgtttgcctg gcggcagtag cgcggtggtc ccacctgacc ccatgccgaa ctcagaagtg
13021 aaacgccgta gcgccgatgg tagtgtgggg actccccatg cgagagtagg gaactgccag
13081 gcatcaaata aaacgaaagg ctcagtcgaa agactgggcc tttcgcccgg gctaattagg
13141 gggtgtcgcc cttattcgac tctatagtga agttcctatt ctctagaaag tataggaact
13201 tctgaagtgg ggtcgactta attaagg
```

These rAAV particles are tested for efficacy in cell culture and then administered to an animal model of LCA10.

### Table 6

### Sequence Listing Free Text

The following information is provided for sequences containing free text under numeric identifier <223>.

| SEQ ID NO: | |
|---|---|
| (containing free text) | Free text under <223> |
| 1 | pAAV ABCA4 3″RTM CMV |
| | synthetic construct |
| 2 | pAAV CEP290 5′RTM CMV CBA |
| | synthetic construct |
| 5 | 5′ splice site with spacer |
| 6 | Splice site for 3′ RTM |
| 7 | Polypyrrimidine tract for 3′ RTM |
| 8 | Spacer for 5′ RTM |
| 9 | Spacer for 3′ RTM |
| 10 | Spacer for 3′ RTM |
| 11 | Polypyrrimidine tract for 3′ RTM |
| 12 | Spacer for 5′ RTM |
| 13 | Spacer for 3′ RTM |
| 14 | Polypyrrimidine tract for 3′ RTM |

All documents listed in this specification, and U.S. provisional application No. 62/257,500, are incorporated herein by reference. While the invention has been described with reference to specific embodiments, it is appreciated that modifications can be made without departing from the spirit of the invention. Such modifications are intended to fall within the scope of the appended claims.

1. A nucleic acid trans-splicing molecule that can replace an exon sequence in a targeted mammalian CEP290 gene carrying a defect or mutation causing an Lebers Congenital Amaurosis type 10 with an exon sequence having the naturally-occurring sequence without the defect or mutation.

2. The nucleic acid trans-splicing molecule according to claim 1, wherein the exon sequence is Exons 1-26.

3. The nucleic acid trans-splicing molecule according to claim 1, wherein the exon sequence is Exons 27-54.

4. The nucleic acid trans-splicing molecule according to claim 1, comprising:
(a) a binding domain (BD) that targets a selected sequence of the CEP290 gene and which binds to the target 5′ to the mutation or defect in the target pre-mRNA;
(b) an optional spacer;
(c) a 3′ splice site, and
(d) a sequence that encodes one or all exons of the CEP290 gene that are 3′ to the binding of the BD to the target, said sequence correcting the defects or mutations in the target gene.

5. The nucleic acid trans-splicing molecule according to claim 4, wherein the gene is CEP290, the binding domain binds to a portion of Intron 26 and the coding sequence encodes Exons 1-26.

6. The nucleic acid trans-splicing molecule according to claim 4, further comprising:
(e) a second binding domain BD sequence that targets a selected sequence of the ocular gene and which binds to the target intron 3′ to the mutation or defect in the target pre-mRNA; and
(f) a 5′ splice site,
wherein the encoding sequence (d) binds to an internal exon(s) of the target, and wherein said molecule is a double trans-splicing molecule.

7. The nucleic acid trans-splicing molecule according to claim 1, comprising in sequential order:
(a) a binding domain BD sequence that targets a selected sequence of an ocular gene and which binds to the target 3′ to the mutation or defect in the target pre-mRNA;
(b) a 5′ splice site;
(c) an optional spacer; and
(d) a sequence that encodes all exons of the ocular gene that are 5′ to the binding of the BD to the target, said sequence correcting the defects or mutations in the target gene.

8. The nucleic acid trans-splicing molecule according to claim 7, wherein the gene is CEP290, the selected intron is Intron 26 and the coding sequence encodes Exons 27-54.

9. The nucleic acid trans-splicing molecule according to claim 1, which is a nucleic acid sequence of up to 3000 bp in length.

10. A proviral plasmid comprising a wildtype 5 adeno-associated virus (AAV) ITR sequence, a promoter comprising an ocular cell-specific promoter/enhancer, a multi-cloning polylinker sequence having inserted therein a nucleic acid sequence of the nucleic acid trans-splicing molecule of claim 1, operatively linked to, and under the regulatory control of, the promoter; and a wildtype 3′ AAV ITR sequence.

11. The proviral plasmid according to claim 10, which is p618 comprising the nucleic acid sequence.

12. A recombinant adeno-associated virus (AAV) comprising a nucleic acid trans-splicing molecule of claim 1.

13. A method of treating an Leber congenital amaurosis type 10 (LCA) disease caused by a defect or mutation in a target CEP290 gene comprising: administering to the photoreceptor cells of a subject having an Leber congenital amaurosis type 10 a composition comprising a recombinant AAV of claim 12.

14. The method according to claim 13, wherein the composition is administered by subretinal injection.

15. The method according to claim 14, wherein the corrected exon sequence is Exons 1-26 or Exons 27-54.

16. A pharmaceutical preparation comprising the recombinant AAV of claim 12 and a pharmaceutically acceptable carrier.
